(19) United States
(12) Patent Application Publication
(10) Pub. No.: US 2024/0161867 A1
(43) Pub. Date: May 16, 2024

(54) OPTIMIZATION OF MODEL-BASED FEATURIZATION AND CLASSIFICATION

(71) Applicant: Grail, LLC, Menlo Park, CA (US)

(72) Inventors: Alexander P. Fields, Burlingame, CA (US); John F. Beausang, Menlo Park, CA (US); Oliver Claude Venn, San Francisco, CA (US); Arash Jamshidi, Redwood City, CA (US); M. Cyrus Maher, San Mateo, CA (US); Qinwen Liu, Fremont, CA (US); Jan Schellenberger, Belmont, CA (US); Joshua Newman, Mountain View, CA (US); Robert Abe Paine Calef, Redwood City, CA (US); Samuel S. Gross, Sunnyvale, CA (US); Frank Chu, San Jose, CA (US); Earl Hubbell, Palo Alto, CA (US)

(21) Appl. No.: 18/511,450

(22) Filed: Nov. 16, 2023

Related U.S. Application Data
(60) Provisional application No. 63/425,889, filed on Nov. 16, 2022.

(51) Int. Cl. G16B 20/20; G16B 40/20; G16H 50/20

(52) U.S. Cl. CPC G16B 20/20; G16B 40/20; G16H 50/20

(57) ABSTRACT

One or more techniques for optimizing cancer classification based on covariate characteristics is disclosed. In a first approach, an analytics system may determine separate cutoff thresholds for positively detecting disease signal for different labels for a covariate characteristic. The system may subdivide training samples based on their labels for the covariate characteristic, to separately determine the cutoff thresholds. In other approaches, the system may train disparate classifiers for each population. The system separates the training samples based on their labels for the covariate characteristic, and separately trains classifiers to generate a signal vector representing an amount of disease signal detected in a sample. The classifiers may be trained on different feature sets as determined based on mutual information gain, genomic region coverage, and healthy activation fraction.

### CROSS-REFERENCE TO RELATED APPLICATIONS

The present application claims the benefit of and priority to U.S. Provisional Application No. 63/425,889 filed on Nov. 16, 2022, which is incorporated by reference in its entirety.

### BACKGROUND

### 1. Field of Art

This disclosure generally relates to model-based featurization and classifiers for predicting disease state from nucleic acid samples.

### 2. Description of the Related Art

DNA methylation plays a role in regulating gene expression. Aberrant DNA methylation has been implicated in many disease processes, including cancer. DNA methylation profiling using methylation sequencing (e.g., whole genome bisulfite sequencing (WGBS)) is increasingly recognized as a valuable diagnostic tool for detection, diagnosis, and/or monitoring of cancer. For example, specific patterns of differentially methylated regions may be useful as molecular markers for various disease states.

### SUMMARY

Cancer classification generally entails applying one or more predictive models to features derived from genetic sequencing data to predict a disease state of an individual. In particular, cancer classification may featurize sequencing data by utilizing tissue models to determine tissue origin of sequence reads pertaining to nucleic acid fragments. The disease state may be a binary prediction indicating detected presence of disease signal generally, or may be a multiclass prediction indicating detected presence of particular disease signals.

One or more techniques may be implemented for optimizing cancer classification based on covariate characteristics. In a first approach, an analytics system may determine separate cutoff thresholds for positively detecting disease signal for different labels for a covariate characteristic. The system may subdivide training samples based on their labels for the covariate characteristic, to separately determine the cutoff thresholds. In other approaches, the system may train disparate classifiers for each population. The system separates the training samples based on their labels for the covariate characteristic, and separately trains classifiers to generate a signal vector representing an amount of disease signal detected in a sample. The classifiers may be trained on different feature sets as determined based on mutual information gain, genomic region coverage, and healthy activation fraction.

Clause 1. A method comprising: obtaining a plurality of training samples from individuals each having one of a plurality of disease states and each associated with one of a plurality of labels for a covariate characteristic, wherein each training sample comprises methylation sequence reads for at least 1,000 cell-free deoxyribonucleic acid (cfDNA) fragments obtained from the individual; generating a first set of training samples and a second set of training samples by subdividing the plurality of training samples; generating, for each training sample, a feature vector based on the methylation sequence reads of the training sample by: for each methylation sequence read of the training sample: applying each of a plurality of tissue models to the methylation sequence reads to determine a likelihood that the methylation sequence read is informative of presence of one disease state associated with the tissue model, assigning the methylation sequence read to one of the disease states with the highest likelihood output by the tissue models, and determining the feature vector based on the methylation sequence reads assigned to each disease state; training, with the feature vectors of the first set of training samples, a classifier to generate a signal vector based on an input feature vector, wherein the signal vector comprises a value for each disease state; applying the cancer classifier to the feature vector of each training sample in the second set of training samples to generate a cancer signal vector for each training sample in the second set of training samples; and for each label of the plurality of labels for the covariate characteristic, determining a cutoff threshold for each disease state based on the signal vectors for the training samples in the second set of training samples with the label.

Clause 2. The method of clause 1, wherein the methylation sequence reads are obtained from a targeted methylation sequencing assay, or a whole genome bisulfite sequencing assay.

Clause 3. The method of any of clauses 1-2, wherein each training sample comprises methylation sequence reads for at least 10,000 cfDNA fragments.

Clause 4. The method of any of clauses 1-3, wherein the first set of training samples and the second set of training samples comprise similar proportions of training samples over the disease states.

Clause 5. The method of any of clauses 1-4, wherein the plurality of disease states includes a non-cancer state and one or more cancer states for one or more cancers of distinct origins.

Clause 6. The method of clause 5, wherein the one or more cancers of distinct origins comprise: breast cancer, uterine cancer, cervical cancer, ovarian cancer, bladder cancer, urothelial cancer of renal pelvis and ureter, renal cancer other than urothelial, prostate cancer, anorectal cancer, colorectal cancer, squamous cell cancer of esophagus, esophageal cancer other than squamous, gastric cancer, hepatobiliary cancer arising from hepatocytes, hepatobiliary cancer arising from cells other than hepatocytes, pancreatic cancer, human-papillomavirus-associated head and neck cancer, head and neck cancer not associated with human papillomavirus, lung adenocarcinoma, small cell lung cancer, squamous cell lung cancer and lung cancer other than adenocarcinoma or small cell lung cancer, neuroendocrine cancer, melanoma, thyroid cancer, sarcoma, multiple myeloma, lymphoma, and leukemia. In some embodiments, the cancer type is additionally selected from a group including brain cancer, vulvar cancer, vaginal cancer, testicular cancer, mesothelioma of the pleura, mesothelioma of the peritoneum, and gallbladder cancer.

Clause 7. The method of any of clauses 1-6, wherein the covariate characteristic is one of: age, status as a smoker, and biological sex.

Clause 8. The method of any of clauses 1-7, further comprising: determining, for each methylation sequence read, with p-value filtering whether the methylation sequence read has an informative methylation pattern, wherein the feature vector for each training sample is generated based on the methylation sequence reads with informative methylation patterns.

Clause 9. The method of any of clauses 1-8, wherein each tissue model of the plurality of tissue models is trained by: obtaining a second plurality of training samples each having one of a plurality of disease states, wherein each training sample comprises methylation sequence reads for cfDNA fragments obtained from the individual; for each tissue model, generating a training dataset comprising at least 10,000 methylation sequence reads of training samples having the disease state associated with the tissue model; and training each tissue model with the associated training dataset to predict a likelihood that a methylation sequence read is informative of presence of the associated disease state.

Clause 10. The method of any of clauses 1-9, wherein each tissue model is one of: a binomial model, an independent sites model, a Markov model, or a mixture model.

Clause 11. The method of any of clauses 1-9, wherein the classifier is a machine-learning model.

Clause 12. The method of any of clauses 1-11, further comprising: obtaining a test sample from a test individual having an unknown disease state and associated with a first label of the plurality of labels for the covariate characteristic, wherein the test sample comprises methylation sequence reads for cfDNA fragments obtained from the test individual; generating a test feature vector based on the methylation sequence reads of the test sample by: for each methylation sequence read of the test sample: applying each of the plurality of tissue models to the methylation sequence reads to determine a likelihood that the methylation sequence read is informative of presence of one disease state associated with the tissue model, assigning the methylation sequence read to one of the disease states with the highest likelihood output by the tissue models, and determining the test feature vector based on the methylation sequence reads assigned to each disease state; applying the classifier to the test feature vector of the test to generate a signal vector for the test sample; and detecting positive disease signal for one or more of the disease states by applying the cutoff thresholds associated with the first label for the covariate characteristic to the signal vector for the test sample.

Clause 13. The method of clause 12, wherein detecting the positive disease signal for one or more of the disease states comprises: for each disease state, applying the cutoff threshold for the disease state to the value in the signal vector for the test sample corresponding to the disease state.

Clause 14. The method of any of clauses 1-13, wherein each individual is further associated with one of a second plurality of labels for a second covariate characteristic; wherein determining the cutoff thresholds comprises determining, for each combination of one label from the plurality of labels for the covariate characteristic and one label from the second plurality of labels for the second covariate characteristic; wherein the test sample is further associated with a second label for the second covariate characteristic; and wherein detecting the positive disease signal for one or more of the disease states comprises applying the cutoff thresholds associated with the combination of the first label for the covariate characteristic and the second label for the second covariate characteristic.

Clause 15. The method of any of clauses 12-14, further comprising reporting the positive disease signal to a healthcare provider for additional workup diagnostic steps.

Clause 16. A method comprising: obtaining a plurality of training samples from individuals each having one of a plurality of disease states and each associated with one of a plurality of labels for a covariate characteristic, wherein each training sample comprises methylation sequence reads for at least 1,000 cell-free deoxyribonucleic acid (cfDNA) fragments obtained from the individual; generating, for each training sample, a feature vector based on the methylation sequence reads of the training sample by: for each methylation sequence read of the training sample: applying each of a plurality of tissue models to the methylation sequence reads to determine a likelihood that the methylation sequence read is informative of presence of one disease state associated with the tissue model, assigning the methylation sequence read to one of the disease states with the highest likelihood output by the tissue models, and determining the feature vector based on the methylation sequence reads assigned to each disease state; generating a set of training samples for each label of the plurality of labels for the covariate characteristic comprising the feature vectors of the training samples associated with the label for the covariate characteristic; for each label, training, with the feature vectors of the set of training samples corresponding to the label, a classifier to generate a signal vector based on an input feature vector, wherein the signal vector comprises a value for each disease state.

Clause 17. The method of clause 16, wherein the methylation sequence reads are obtained from a targeted methylation sequencing assay, or a whole genome bisulfate sequencing assay.

Clause 18. The method of any of clauses 16-17, wherein each training sample comprises methylation sequence reads for at least 10,000 cfDNA fragments.

Clause 19. The method of any of clauses 16-18, wherein the plurality of disease states includes a non-cancer state and one or more cancer states for one or more cancers of distinct origins.

Clause 20. The method of clause 19, wherein the one or more cancers of distinct origins comprise: breast cancer, uterine cancer, cervical cancer, ovarian cancer, bladder cancer, urothelial cancer of renal pelvis and ureter, renal cancer other than urothelial, prostate cancer, anorectal cancer, colorectal cancer, squamous cell cancer of esophagus, esophageal cancer other than squamous, gastric cancer, hepatobiliary cancer arising from hepatocytes, hepatobiliary cancer arising from cells other than hepatocytes, pancreatic cancer, human-papillomavirus-associated head and neck cancer, head and neck cancer not associated with human papillomavirus, lung adenocarcinoma, small cell lung cancer, squamous cell lung cancer and lung cancer other than adenocarcinoma or small cell lung cancer, neuroendocrine cancer, melanoma, thyroid cancer, sarcoma, multiple myeloma, lymphoma, and leukemia. In some embodiments, the cancer type is additionally selected from a group including brain cancer, vulvar cancer, vaginal cancer, testicular cancer, mesothelioma of the pleura, mesothelioma of the peritoneum, and gallbladder cancer.

Clause 21. The method of any of clauses 16-20, wherein the covariate characteristic is one of: age, status as a smoker, and biological sex.

Clause 22. The method of any of clauses 16-21, further comprising: determining, for each methylation sequence read, with p-value filtering whether the methylation sequence read has an informative methylation pattern, wherein the feature vector for each training sample is generated based on the methylation sequence reads with informative methylation patterns.

Clause 23. The method of any of clauses 16-22, wherein each tissue model of the plurality of tissue models is trained by: obtaining a second plurality of training samples each having one of a plurality of disease states, wherein each training sample comprises methylation sequence reads for cfDNA fragments obtained from the individual; for each tissue model, generating a training dataset comprising at least 10,000 methylation sequence reads of training samples having the disease state associated with the tissue model; and training each tissue model with the associated training dataset to predict a likelihood that a methylation sequence read is informative of presence of the associated disease state.

Clause 24. The method of any of clauses 16-23, wherein each tissue model is one of: a binomial model, an independent sites model, a Markov model, or a mixture model.

Clause 25. The method of any of clauses 16-24, wherein each classifier is a machine-learning model.

Clause 26. The method of any of clauses 16-25, further comprising: obtaining a test sample from a test individual having an unknown disease state and associated with a first label of the plurality of labels for the covariate characteristic, wherein the test sample comprises methylation sequence reads for cfDNA fragments obtained from the test individual; generating a test feature vector based on the methylation sequence reads of the test sample by: for each methylation sequence read of the test sample: applying each of the plurality of tissue models to the methylation sequence reads to determine a likelihood that the methylation sequence read is informative of presence of one disease state associated with the tissue model, assigning the methylation sequence read to one of the disease states with the highest likelihood output by the tissue models, and determining the test feature vector based on the methylation sequence reads assigned to each disease state; applying the classifier corresponding to the first label to the test feature vector of the test to generate a signal vector for the test sample; and detecting positive disease signal for one or more of the disease states based on the signal vector for the test sample.

Clause 27. The method of clause 26, wherein detecting the positive disease signal for one or more of the disease states comprises: identifying the disease state with the largest value in the signal vector as the disease state with the detected positive disease signal.

Clause 28. The method of any of clauses 16-27, wherein each individual is further associated with one of a second plurality of labels for a second covariate characteristic; wherein generating the sets of training samples comprises generating, for each combination of one label from the plurality of labels for the covariate characteristic and one label from the second plurality of labels for the second covariate characteristic, a set of training samples; wherein training the classifiers comprises training a classifier for each combination with the feature vectors from the set of training samples corresponding to the combination; wherein the test sample is further associated with a second label for the second covariate characteristic; and wherein applying the classifier comprises applying the classifier trained for the combination of the first label and the second label.

Clause 29. The method of any of clauses 12-14, further comprising reporting the positive disease signal to a healthcare provider for additional workup diagnostic steps.

Clause 30. A method comprising: obtaining a plurality of training samples from individuals each having one of a plurality of disease states and each associated with one of a plurality of labels for a covariate characteristic, wherein each training sample comprises methylation sequence reads for at least 1,000 cell-free deoxyribonucleic acid (cfDNA) fragments obtained from the individual; generating, for each training sample, a feature vector based on the methylation sequence reads of the training sample by: for each of a plurality of genomic regions, determining a methylation feature value based on one or more methylation sequence reads of the training sample overlapping the genomic region, and determining the feature vector based on the methylation feature values across the plurality of genomic regions; generating a set of training samples for each label of the plurality of labels for the covariate characteristic comprising the feature vectors of the training samples associated with the label for the covariate characteristic; for each label: determining a mutual information score for each genomic region based on the feature vectors of the set of training samples corresponding to the label; ranking the genomic regions based on the mutual information scores; selecting a set of features from the ranked genomic regions; modifying the feature vectors to include methylation feature values for the set of features; and training a classifier with the modified feature vectors to generate a signal vector based on an input feature vector, wherein the signal vector comprises a value for each disease state.

Clause 31. The method of clause 30, wherein the methylation sequence reads are obtained from a targeted methylation sequencing assay, or a whole genome bisulfite sequencing assay.

Clause 32. The method of any of clauses 30-31, wherein each training sample comprises methylation sequence reads for at least 10,000 cfDNA fragments.

Clause 33. The method of any of clauses 30-32, wherein each genomic region comprises one or more CpG sites.

Clause 34. The method of any of clauses 30-33, wherein the methylation feature value is a methylation density at the genomic region.

Clause 35. The method of any of clauses 30-33, further comprising: determining, for each methylation sequence read, with p-value filtering whether the methylation sequence read has an informative methylation pattern, wherein the feature vector for each training sample is generated based on the methylation sequence reads with informative methylation patterns.

Clause 36. The method of clause 35, wherein the methylation feature value is a count of methylation sequence reads overlapping the genomic region.

Clause 37. The method of any of clauses 30-36, wherein the plurality of disease states includes a non-cancer state and one or more cancer states for one or more cancers of distinct origins.

Clause 38. The method of clause 37, wherein the one or more cancers of distinct origins comprise: breast cancer, uterine cancer, cervical cancer, ovarian cancer, bladder cancer, urothelial cancer of renal pelvis and ureter, renal cancer other than urothelial, prostate cancer, anorectal cancer, colorectal cancer, squamous cell cancer of esophagus, esophageal cancer other than squamous, gastric cancer, hepatobiliary cancer arising from hepatocytes, hepatobiliary cancer arising from cells other than hepatocytes, pancreatic cancer, human-papillomavirus-associated head and neck cancer, head and neck cancer not associated with human papillomavirus, lung adenocarcinoma, small cell lung cancer, squamous cell lung cancer and lung cancer other than adenocarcinoma or small cell lung cancer, neuroendocrine cancer, melanoma, thyroid cancer, sarcoma, multiple myeloma, lymphoma, and leukemia. In some embodiments, the cancer type is additionally selected from a group including brain cancer, vulvar cancer, vaginal cancer, testicular cancer, mesothelioma of the pleura, mesothelioma of the peritoneum, and gallbladder cancer.

Clause 38. The method of any of clauses 30-37, wherein the covariate characteristic is one of: age, status as a smoker, and biological sex.

Clause 39. The method of any of clauses 30-38, wherein determining for each label the mutual information score for each genomic region comprises: determining a pairwise information gain for each pairwise combination of disease states based on discriminatory power of the genomic region to classify between the two disease states; and determining the mutual information score by combining the pairwise information gains across the pairwise combinations of disease states.

Clause 40. The method of any of clauses 30-39, further comprising: for each label: determining a coverage for each genomic region based on a sequencing depth of the set of training samples corresponding to the label; and excluding one or more genomic regions from selection for the set of features based on the coverage being below a threshold.

Clause 41. The method of any of clauses 30-40, further comprising: for each label: determining, for each genomic region, an activation score for a non-cancer disease state indicating a number of training samples having the non-cancer disease state showing activation of the genomic region; and excluding one or more genomic regions from selection for the set of features based on activation score being above a threshold.

Clause 42. The method of any of clauses 30-41, wherein the set of features selected for each label is determined to optimize precision of the classifier.

Clause 43. The method of any of clauses 30-42, wherein one set of features for one label is different than another set of features for another label.

Clause 44. The method of clause 43, wherein the one set of features comprises one or more different features than the other set of features.

Clause 45. The method of clause 43, wherein the one set of features comprises a different number of features than the other set of features.

Clause 46. The method of any of clauses 30-45, wherein each classifier is a machine-learning model.

Clause 47. The method of any of clauses 16-25, further comprising: obtaining a test sample from a test individual having an unknown disease state and associated with a first label of the plurality of labels for the covariate characteristic, wherein the test sample comprises methylation sequence reads for cfDNA fragments obtained from the test individual; generating a test feature vector based on the methylation sequence reads of the test sample by: for each of the plurality of genomic regions, determining a methylation feature value based on one or more methylation sequence reads of the test sample overlapping the genomic region, and determining the test feature vector based on the methylation feature values across the plurality of genomic regions; applying the classifier corresponding to the first label to the test feature vector of the test to generate a signal vector for the test sample; and detecting positive disease signal for one or more of the disease states based on the signal vector for the test sample.

Clause 48. The method of clause 47, wherein detecting the positive disease signal for one or more of the disease states comprises: identifying the disease state with the largest value in the signal vector as the disease state with the detected positive disease signal.

Clause 49. The method of any of clauses 12-14, further comprising reporting the positive disease signal to a healthcare provider for additional workup diagnostic steps.

Clause 50. A method for optimizing feature selection, comprising: obtaining, via an analytics system, a plurality of sequence reads from a sample; generating, via the analytics system, a plurality of features based on the plurality of sequence reads; generating, via the analytics system, one or more of activation fractions, mutual information scores, or coverages based on the plurality of sequence reads; determining, via the analytics system, a rank of the plurality of features based on the one or more activation fractions, mutual information scores, or coverages; and selecting, via the analytics system, a subset of features from the plurality of features based on the rank of the plurality of features.

Clause 51. The method of clause 50, wherein the sample comprises a cell free nucleic acid sample.

Clause 52. The method of any of clauses 50-51, wherein the plurality of features comprise sequence read counts of sequence reads that exceed a ratio threshold value.

Clause 53. The method of any of clauses 50-52, wherein generating the plurality of features comprises determining rates of methylation for a plurality of CpG sites within the plurality of sequence reads.

Clause 54. The method of any of clauses 50-53, wherein the activation fractions comprise non-cancer activation fractions.

Clause 55. The method of any of clauses 50-54, wherein a mutual information score of the mutual information scores corresponding to a feature of the plurality of features is inversely proportional to a rank of the feature.

Clause 56. The method of any of clauses 50-55, wherein a lower coverage corresponds to a noisier feature.

Clause 57. The method of any of clauses 50-56, further comprising training at least one classifier based on the subset of features, the at least one classifier trained to predict a presence or absence of the disease, a disease type, and/or a disease tissue of origin.

Clause 58. The method of clause 57, wherein the at least one classifier is trained to determine the disease tissue of origin based on one or more characteristics.

Clause 59. The method of clause 58, wherein the one or more characteristics comprise at least one of age, a status as a smoker, and sex.

Clause 60. The method of any of clauses 50-59, further comprising: determining a first activation fraction for a disease positive prediction; and determining a second activation fraction for a disease negative prediction; wherein the subset of features is selected at least in part by minimizing the difference between the first activation fraction and the second activation fraction.

Clause 61. The method of any of clauses 50-60, further comprising: generating a set of training data corresponding to the subset of features, the training data generated from sequence data labeled with a disease type or tissue of origin or labeled as healthy; training a machine-learned model using the generated set of training data, the machine-learned model configured to predict a disease state and type based on sequence data corresponding to the subset of features.

Clause 62. A non-transitory computer-readable storage medium storing instructions that, when executed by a computer processor, cause the processor to perform the method of any of clauses 1-61.

Clause 63. A system comprising: the computer-processor; and the non-transitory computer-readable storage medium of clause 62.

Clause 64. A treatment kit comprising: a collection vessel for storing a biological sample comprising cfDNA fragments; optionally, one or more reagents for isolating the cfDNA fragments from the biological sample; optionally, a sequencing panel comprising probes for targeting particular genomic regions; and the non-transitory computer-readable storage medium of clause 62.

### BRIEF DESCRIPTION OF THE DRAWINGS

FIG. 1A is a flowchart of a method for generating a classifier to predict disease state, according to various embodiments.

FIG. 1B is a flowchart of a method for generating a classifier to predict disease state, according to various embodiments.

FIG. 2A illustrates a flowchart of devices for sequencing nucleic acid samples according to one embodiment.

FIG. 2B is block diagram of an analytics system for processing sequence reads, according to various embodiments.

FIG. 3 is a flowchart describing a process of sequencing nucleic acids, according to various embodiments.

FIG. 4A is an illustration of a part of the process of FIG. 3 of sequencing nucleic acids to obtain a methylation information and methylation state vectors, according to various embodiments.

FIG. 4B illustrates generation of a data structure for a control group, according to various embodiments.

FIG. 4C illustrates a flowchart describing a process of determining informative fragments from a sample, according to various embodiments.

FIG. 5 is an illustration of blocks of a reference genome, according to various embodiments.

FIG. 6 is an illustration of a process of determining features to train a classifier, according to various embodiments.

FIGS. 7A, 7B, 7C, 7D, 7E, and 7F include confusion matrices indicating accuracy of classifiers, according to various embodiments.

FIG. 8 is a flowchart of a method for model-based featurization, according to various embodiments.

FIGS. 9A and 9B illustrate sensitivity of tissue of origin classifiers, according to an embodiment.

FIGS. 10A and 10B illustrate sensitivity of tissue of origin classifiers at different cancer stages, according to an embodiment.

FIG. 11 illustrates a performance grid representing the accuracy of tissue of origin localization, according to an embodiment.

FIG. 12 illustrates accuracy and sensitivity of a tissue of origin classifier at different cancer stages, according to embodiment.

FIGS. 13A and 13B illustrates ROC curves for a tissue of origin classifier, according to an embodiment.

FIG. 14 depicts a data flow diagram for training models, according to various embodiments.

FIG. 15 illustrates a precision-recall curve for indeterminate call thresholds, according to various embodiments.

FIG. 16 is a flowchart of a method for determining a probability that a sample has a disease state according to various embodiments.

FIG. 17 illustrates performance gain in sensitivity of a multilayer perceptron model according to an embodiment.

FIG. 18 illustrates experimental results of a multilayer perceptron model in determining tissue of origin according to an embodiment.

FIG. 19 illustrates experimental results of a multilayer perceptron model in determining tissue of origin by cancer stage according to an embodiment.

FIG. 20 illustrates experimental results of a multilayer perceptron model across types of cancers according to an embodiment.

FIG. 21 illustrates a graph of cancer type likelihood for non-cancer samples above 95% specificity.

FIG. 22 illustrates a graph of methylation sequencing data of non-cancer samples and hematological sub-type cancer samples.

FIG. 23A illustrates a flowchart describing a process of determining a binary threshold cutoff for binary cancer classification, in accordance with one or more embodiments.

FIG. 23B illustrates a flowchart describing a process of thresholding a tissue of origin label for determining a binary threshold cutoff for binary cancer classification, in accordance with one or more embodiments.

FIGS. 24A and 24B illustrates confusion matrices demonstrating performance of a trained cancer tissue of origin classifier with additional hematological cancer sub-types.

FIGS. 25A and 25B illustrate graphs showing cancer prediction accuracy for cancer classifiers with and without adjusting a threshold cutoff for numerous cancer types over stages of cancer.

FIG. 26A depicts a receiver operator curve (ROC) showing the sensitivity and specificity of cancer detection using methylation data for the target genomic regions of Assay Panel A.

FIG. 26B is a confusion matrix depicting the accuracy of cancer type classifications for subjects determined to have cancer using methylation data for the target genomic regions of Assay Panel A.

FIG. 27A depicts a receiver operator curve (ROC) showing the sensitivity and specificity of cancer detection using methylation data for the target genomic regions of Assay Panel B.

FIG. 27B is a confusion matrix depicting the accuracy of cancer type classifications for subjects determined to have cancer using methylation data for the target genomic regions of Assay Panel B.

FIG. 28 shows classifier performance for a proprietary cancer assay panel (Assay Panel C), in accordance with an embodiment.

FIGS. 29A & 29B show tissue of origin (TOO) confusion matrices representing the accuracy of cancer tissue of origin localization for Assay Panel C, according to an embodiment.

FIG. 30 show classifier sensitivity performance in individual tumors by stage for Assay Panel C, in accordance an embodiment.

FIG. 31 shows tissue of origin accuracy of multiple iterations of trained models in accordance to various embodiments.

FIG. 32 illustrates a process for stratifying hematological signals into two strata, in accordance with various embodiments.

FIG. 33 illustrates predicted and actual labels of cancer signal origin for a sample of male and female individuals, according to one embodiment.

FIG. 34 illustrates predicted and actual labels of cancer signal origin for a sample of male individuals, according to one embodiment.

FIG. 35 illustrates predicted and actual labels of cancer signal origin for a sample of female individuals, according to one embodiment.

FIG. 36 illustrates experimental results of fragment coverage versus feature rank, according to one embodiment.

FIG. 37A illustrates experimental results of fragment coverage versus non-cancer activation fractions, according to one embodiment.

FIG. 37B illustrates experimental results of non-cancer activation fraction versus feature rank, according to one embodiment.

FIG. 38A illustrates experimental results of non-cancer activation fraction versus feature weight, according to one embodiment.

FIG. 38B illustrates another view of the experimental results shown in FIG. 38A, according to one embodiment.

FIG. 39A illustrates experimental results of activation fraction versus coverage, according to one embodiment.

FIG. 39B illustrates another view of the experimental results shown in FIG. 39A, according to one embodiment.

FIGS. 40-42 illustrate experimental results of various thresholding methods described herein, according to various embodiments.

### DETAILED DESCRIPTION

Early detection and classification of cancer is an important technology. Being able to detect cancer before it becomes symptomatic is beneficial to all parties involved, including patients, doctors, and loved ones. For patients, early cancer detection allows them a greater chance of a beneficial outcome; for doctors, early cancer detection allows more pathways of treatment that may lead to a beneficial outcome; for loved ones, early cancer detection increases the likelihood of not losing friends and family to the disease.

Recently, early cancer detection technology has progressed towards analyzing genetic fragments (e.g., DNA), e.g., in a person's blood, to determine if any of those genetic fragments originate from cancer cells. These new techniques allow doctors to identify a cancer presence in a patient that may not be detectable otherwise, e.g., in conventional screening processes. For instance, consider the example of a person at high risk for breast cancer. Traditionally, this person will regularly visit their doctor for a mammogram, which creates an image of their breast tissue (e.g., taking x-ray images) that a doctor uses to identify cancerous tissue. Unfortunately, with even the highest resolution mammograms, doctors are only able to identify tumors once they are approximately a millimeter in size. This means that the cancer has been present for some time in the person and has gone undiagnosed and untreated. Visual determinations like this are typical for most cancers—that is, only identifiable once it has grown to a sufficient size to be detected by some sort of imaging technology.

Cancer detection using analysis of genetic fragments in a patient's, e.g., blood alleviates this issue. To illustrate, cancer cells will start sloughing DNA fragments into a person's bloodstream as soon as they form. This occurs when there are very few of the cancer cells, and before they would be visible with imaging techniques. With the appropriate methods, therefore, a system that analyzes DNA fragments in the bloodstream could identify cancer presence in a person based on sloughed cancer DNA fragments, and, more importantly, the system could do so before the cancer is identifiable using more traditional cancer detection techniques.

Cancer detection based on the analysis of DNA fragments is enabled by next-generation sequencing (“NGS”) techniques. NGS, broadly, is a group of technologies that allows for high throughput sequencing of genetic material. As discussed in greater detail herein, NGS largely consists of (1) sample preparation, (2) DNA sequencing, and (3) data analysis. Sample preparation is the laboratory methods necessary to prepare DNA fragments for sequencing, sequencing is the process of reading the ordered nucleotides in the samples, and data analysis is processing and analyzing the genetic information in the sequencing data to identify cancer presence.

While these steps of NGS may help enable early cancer detection, they also introduce their own complex, detrimental problems to cancer detection and, therefore, any improvements to sample preparation, DNA sequencing, and/or data analysis, including the pre-processing, algorithmic processing, and summary or presentation of predications or conclusions, results in an improvement to cancer detection technologies and early cancer detection more generally.

To illustrate, as an example, problems introduced in (1) sample preparation include DNA sample quality, sample contamination, fragmentation bias, and accurate indexing. Remedying these problems would yield better genetic data for cancer detection.

Similarly, problems introduced in (2) sequencing include, for example, errors in accurate transcribing of fragments (e.g., reading an “A” instead of a “C”, etc.), incorrect or difficult fragment assembly and overlap, disparate coverage uniformity, sequencing depth vs. cost vs. specificity, and insufficient sequencing length. Again, remedying any of these problems would yield improved genetic data for cancer detection.

The problems in (3) data analysis are the most daunting and complex. The introduced challenges stem from the vast amounts of data created by NGS sequencing techniques. Sequencing data for a single sample can be on the order of hundreds of thousands (up to millions) of sequence reads, amounting to terabytes of data. Multiply that by the thousands (up to tens of thousands) of samples which are collected for use in training of the analytical models. Effectively and efficiently analyzing that amount of data is both procedurally and computationally demanding. For instance, analyzing NGS sequencing involves several baseline processing steps such as, e.g., aligning reads to one another, aligning and mapping reads to a reference genome, de-duping duplicative reads, detecting contamination of a sample, identifying and calling variant genes, identifying and calling abnormally methylated genes, generating functional annotations, etc. Performing any of these processes on terabytes of genetic data is computationally expensive for even the most powerful of computer architectures, and completely impossible for a normal human mind. Additionally, with the genetic sequencing data derived from the error-prone processes of sample preparation and sequence reading, large portions of the resulting genetic data may be low-quality or unusable for cancer identification. For example, large amounts of the genetic data may include contaminated samples, transcription errors, mismatched regions, overrepresented regions, etc. and may be unsuitable for high accuracy cancer detection. Identifying and accounting for low quality genetic data across the vast amount of genetic data obtained from NGS sequencing is also procedurally and computationally rigorous to accomplish and is also not practically performable by a human mind. Overall, any process created that leads to more efficient processing of large array sequencing data would be an improvement to cancer detection using NGS sequencing. Moreover, such processes were crafted as a solution to the various hurdles created in NGS sequences, and as such are non-routine and unconventional activity in the technical field of endeavor.

Particularly, under (3) data analysis, accurate identification of informative DNA from NGS data to identify a cancer presence is also a difficult task-at-hand. To be effective, algorithms are sought to compensate for, e.g., errors generated by sample preparation and sequencing, and to overcome the large-scale data analysis problems accompanying NGS techniques. That is, designing a machine learning model or models, or other computational processing algorithms, that enable early cancer detection based on next generation sequencing techniques must be configured to account for the problems that those techniques create. Some of those techniques and models are discussed hereinbelow and particular improvements to state-of-the-art techniques and models are further discussed. Furthermore, such techniques are non-routine and unconventional activity in the technical field of endeavor.

One particular challenge arises when predicting presence of cancer signal in individuals with disparate backgrounds. For example, the likelihood of a biological female developing breast cancer is a lot higher than the likelihood of a biological male. Or, in another example, the likelihood of a non-smoker developing lung cancer is a lot lower than the likelihood of a heavy smoker. Accounting for such disparities in statistics can lead to improved predictions, thereby enabling more accurate diagnostics by a healthcare provider. The present disclosure aims address this challenge through one or more different approaches. In some embodiments, the system trains one cancer classifier broadly applied to all samples, but tailors the cutoff thresholds for positively detecting presence of one or more disease states to each subpopulation, as defined by one or more covariate characteristics. In other embodiments, the system trains disparate classifiers for each subpopulation. Training may differ according to the training samples used, the features evaluated, or some combination thereof.

The training of the machine-learned models described herein (such as the one or more cancer classifiers, the tissue models, and other models referenced herein) include the performance of one or more non-mathematical operations or implementation of non-mathematical functions at least in part by a machine or computing system, examples of which include but are not limited to data loading operations, data storage operations, data toggling or modification operations, non-transitory computer-readable storage medium modification operations, metadata removal or data cleansing operations, data compression operations, modification operations, image modification operations, noise application operations, noise removal operations, and the like. Accordingly, the training of the machine-learned models described herein may be based on or may involve mathematical concepts, but is not simply limited to the performance of a mathematical calculation, a mathematical operation, or an act of calculating a variable or number using mathematical methods.

Likewise, it should be noted that the training of the models describes herein cannot be practically performed in the human mind alone. The models are innately complex including vast amounts of weights and parameters associated through one or more complex functions. For example, the number of weights in a model may be on the order of thousands, tens of thousands, hundreds of thousands, millions, or billions. Training may entail utilizing training samples on the order of thousands, tens of thousands, hundreds of thousands, millions, or billions. Training and/or deployment of such models involve so great a number of operations that it is not feasibly performable by the human mind alone, nor with the assistance of pen and paper. In such embodiments, the operations may number in the hundreds, thousands, tens of thousands, hundreds of thousands, millions, billions, or trillions. Accordingly, such models are necessarily rooted in computer-technology for their implementation and use.

### I. DEFINITIONS

Unless defined otherwise, all technical and scientific terms used herein have the meaning commonly understood by a person skilled in the art to which this description belongs. As used herein, the following terms have the meanings ascribed to them below.

The term “individual” can refer to any living organism, such as a human individual or an animal individual. The term “healthy individual” refers to an individual presumed to not have a cancer or disease.

The term “subject” refers to an individual whose DNA is being analyzed. A subject may be a test subject whose DNA is be evaluated using whole genome sequencing or a targeted panel as described herein to evaluate whether the person has a disease state (e.g., cancer, type of cancer, or cancer tissue of origin). A subject may also be part of a control group known not to have cancer or another disease. A subject may also be part of a cancer or other disease group known to have cancer or another disease. Control and cancer/disease groups may be used to assist in designing or validating the targeted panel.

The term “reference sample” refers to a sample obtained from a subject with a known disease state.

The term “training sample” refers to a sample obtained from a known disease state that can be used to generate sequence reads. Training samples may be applied to probability models to generate features that can be utilized for disease state classification.

The term “test sample” refers to a sample that may have an unknown disease state.

The term “sequence read” refers to a nucleotide sequence read from a sample obtained from an individual. Sequence reads may be generated from nucleic acid fragments in the sample. A sequence read can be a collapsed sequence read generated from a plurality of sequence reads derived from a plurality of amplicons from a single original nucleic acid molecule. In some embodiments, the sequence read can be a deduplicated sequence read. Sequence reads can be obtained through various methods known in the art.

The term “disease state” refers to presence or non-presence of a disease, a type of disease, and/or a disease tissue of origin. For example, in one embodiment, the present disclosure provides methods, systems, and non-transitory computer readable medium for detecting cancer (i.e., presence or absence of cancer), a type of cancer, or a cancer tissue of origin.

The term “tissue of origin” or “TOO” or “Cancer Signal Origin” refers to the organ, organ group, body region or cell type from which a disease state may arise or originate. For example, the identification of a tissue of origin or cancer cell type typically allows to identify appropriate next steps to further diagnose, stage, and decide on treatment.

The term “methylation” as used herein refers to a chemical process by which a methyl group is added to a DNA molecule. Two of DNA's four bases, cytosine (“C”) and adenine (“A”) can be methylated. For example, a hydrogen atom on the pyrimidine ring of a cytosine base can be converted to a methyl group, forming 5-methylcytosine. Methylation tends to occur at dinucleotides of cytosine and guanine referred to herein as “CpG sites.” In other instances, methylation may occur at a cytosine not part of a CpG site or at another nucleotide that is not cytosine; however, these are rarer occurrences. In this present disclosure, methylation is discussed in reference to CpG sites for the sake of clarity. However, the principles described herein are equally applicable for the detection of methylation in a non-CpG context, including non-cytosine methylation. For example, Adenine methylation has been observed in bacteria, plant and mammalian DNA, although it has received considerably less attention.

In such embodiments, the wet laboratory assay used to detect methylation may vary from those described herein as well known in the art. Further, the methylation state vectors may contain elements that are generally vectors of sites where methylation has or has not occurred (even if those sites are not CpG sites specifically). With that substitution, the remainder of the processes described herein are the same, and consequently the inventive concepts described herein are applicable to those other forms of methylation.

The term “CpG site” refers to a region of a DNA molecule where a cytosine nucleotide is followed by a guanine nucleotide in the linear sequence of bases along its 5′ to 3′ direction. “CpG” is a shorthand for 5′-C-phosphate-G-3′ that is cytosine and guanine separated by only one phosphate group; phosphate links any two nucleotides together in DNA. Cytosines in CpG dinucleotides can be methylated to form 5-methylcytosine.

The term “methylation site” refers to a single site of a DNA molecule where a methyl group can be added. “CpG” sites are the most common methylation site, but methylation sites are not limited to CpG sites. For example, DNA methylation may occur in cytosines in CHG and CHH, where H is adenine, cytosine or thymine. Cytosine methylation in the form of 5-hydroxymethylcytosine may also assessed (see, e.g., WO 2010/037001 and WO 2011/127136, which are incorporated herein by reference), and features thereof, using the methods and procedures disclosed herein. The term “hypomethylated” or “hypermethylated” refers to a methylation status of a DNA molecule containing multiple CpG sites (e.g., more than 3, 4, 5, 6, 7, 8, 9, 10, etc.) where a high percentage of the CpG sites (e.g., more than 80%, 85%, 90%, or 95%, or any other percentage within the range of 50%-100%) are unmethylated or methylated, respectively.

The term “cell free deoxyribonucleic nucleic acid,” “cell free DNA,” or “cfDNA” refers to deoxyribonucleic acid fragments that circulate in bodily fluids such blood, sweat, urine, or saliva and originate from one or more healthy cells and/or from one or more cancer cells.

The term “circulating tumor DNA” or “ctDNA” refers to deoxyribonucleic acid fragments that originate from tumor cells or other types of cancer cells, which may be released into an individual's bodily fluids such blood, sweat, urine, or saliva as result of biological processes such as apoptosis or necrosis of dying cells or actively released by viable tumor cells.

### II. OVERVIEW OF METHOD

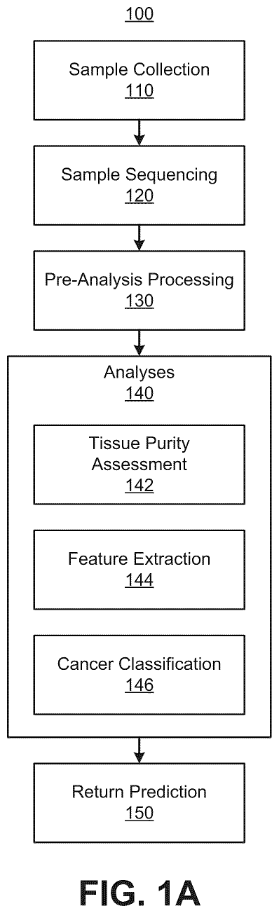

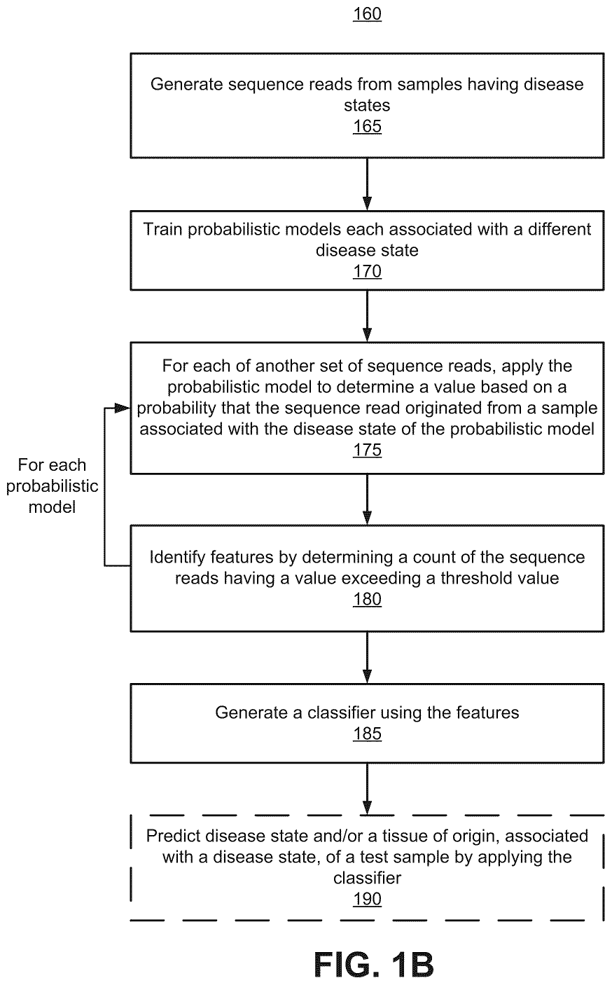

FIG. 1A is an exemplary flowchart describing an overall workflow 100 of cancer classification of a sample, according to one or more embodiments. The workflow 100 is by one or more entities, e.g., including a healthcare provider, a sequencing device, an analytics system, etc. Objectives of the workflow include detecting and/or monitoring cancer in individuals. From a healthcare standpoint, the workflow 100 can serve to supplement other existing cancer diagnostic tools. The workflow 100 may serve to provide early cancer detection and/or routine cancer monitoring to better inform treatment plans for individuals diagnosed with cancer. The overall workflow 100 may include additional/fewer steps than those shown in FIG. 1.

A healthcare provider performs sample collection 110. An individual to undergo cancer classification visits their healthcare provider. The healthcare provider collects the sample for performing cancer classification. Examples of biological samples include, but are not limited to, tissue biopsy, blood, whole blood, plasma, serum, urine, cerebrospinal fluid, fecal, saliva, sweat, tears, pleural fluid, pericardial fluid, or peritoneal fluid of the subject. The sample includes genetic material belonging to the individual, which may be extracted and sequenced for cancer classification. Once the sample is collected, the sample is provided to a sequencing device. Along with the sample, the healthcare provider may collect other information relating to the individual, e.g., biological sex, age, ethnicity, smoking status, any prior diagnoses, etc.

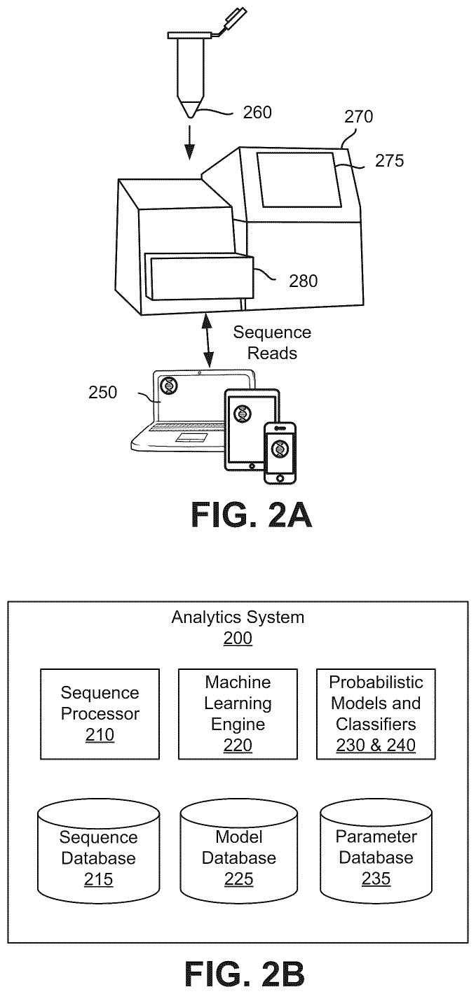

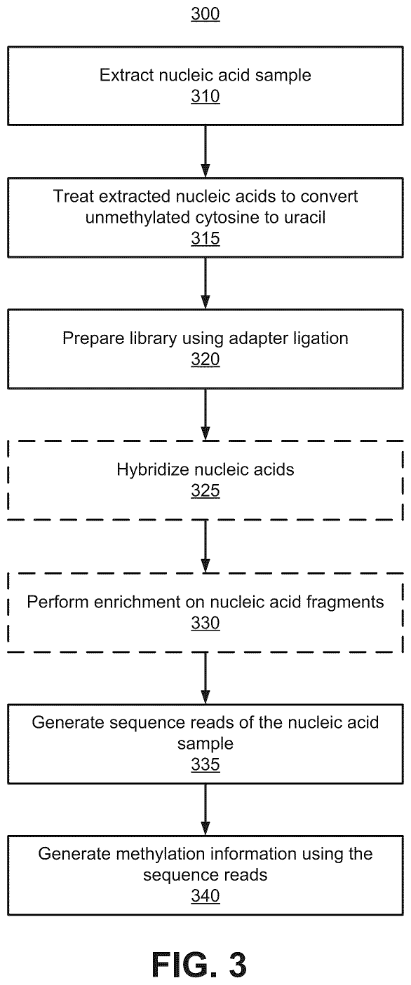

A sequencing device performs sample sequencing 120. A lab clinician may perform one or more processing steps to the sample in preparation of sequencing. Once prepared, the clinician loads the sample in the sequencing device. An example of devices utilized in sequencing is further described in conjunction with FIGS. 2A & 2B. The sequencing device generally extracts and isolates fragments of nucleic acid that are sequenced to determine a sequence of nucleobases corresponding to the fragments. Sequencing may also include amplification of nucleic material. Different sequencing processes include Sanger sequencing, fragment analysis, and next-generation sequencing. Sequencing may be whole-genome sequencing or targeted sequencing with a target panel. In the context of DNA methylation, bisulfite sequencing (e.g., further described in FIGS. 3A & 3B) can determine methylation status through bisulfite conversion of unmethylated cytosines at CpG sites. Sample sequencing 120 yields sequences for a plurality of nucleic acid fragments in the sample. In one or more embodiments, the sequences may include methylation state vectors, wherein each methylation state vector describes the methylation statuses for CpG sites on a fragment.

An analytics system performs pre-analysis processing 130. An example analytics system is described in FIG. 2B. Pre-analysis processing 130 may include, but not limited to, de-duplication of sequence reads, determining metrics relating to coverage, determining whether the sample is contaminated, removal of contaminated fragments, calling sequencing error, etc.

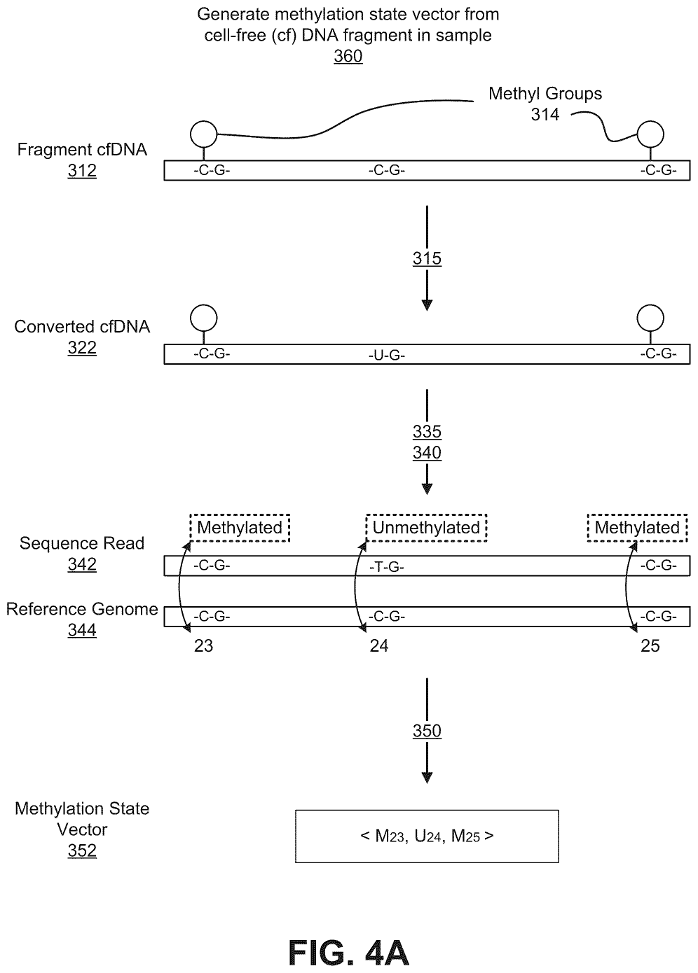

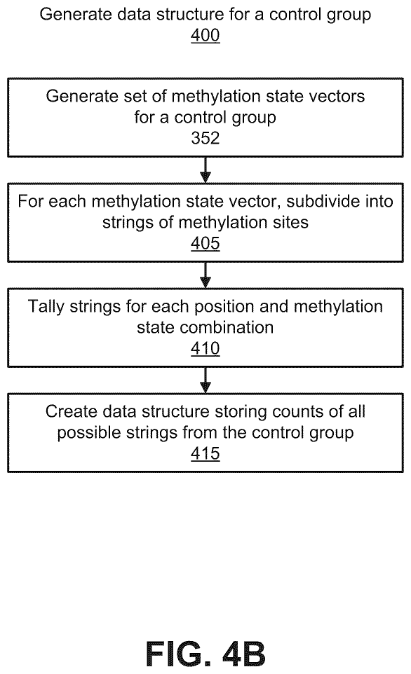

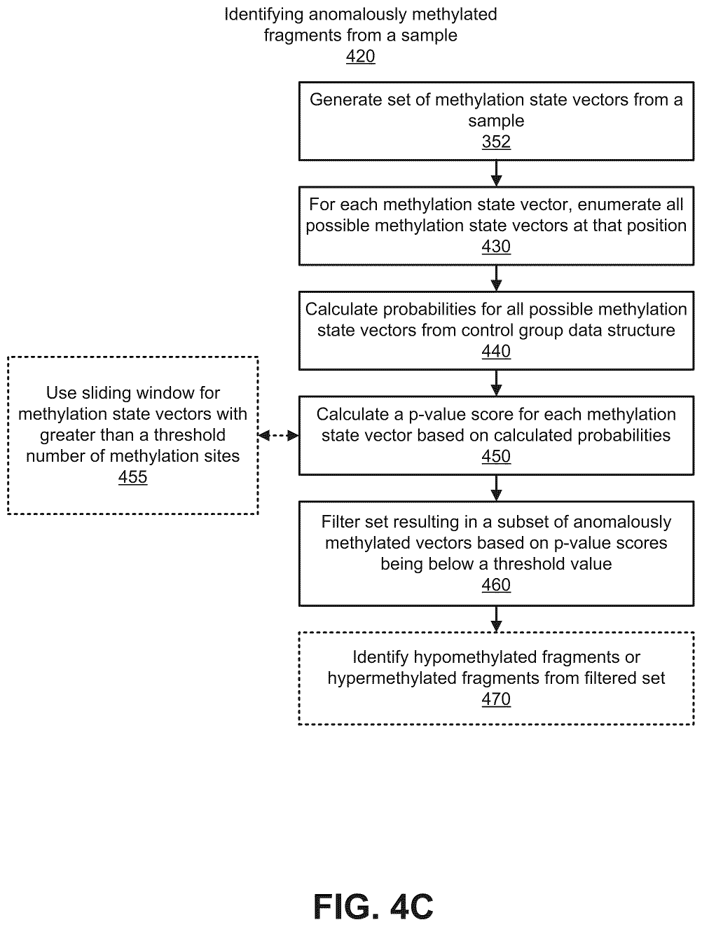

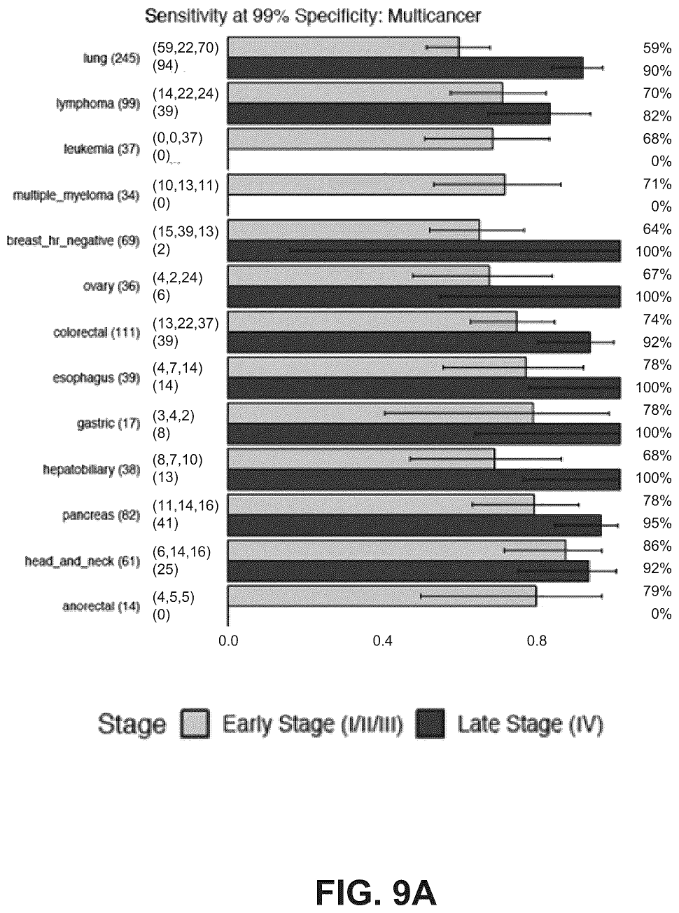

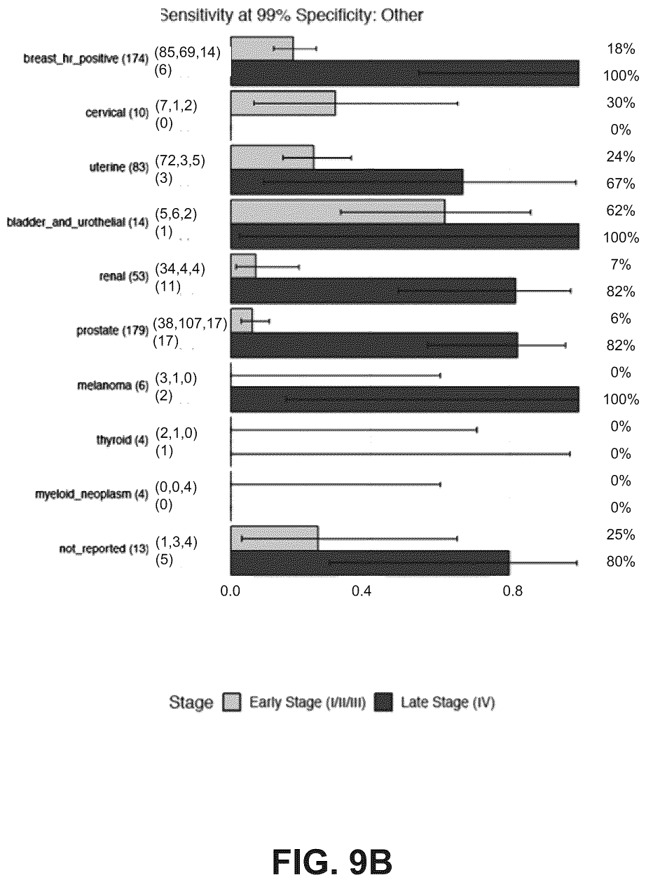

The analytics system performs one or more analyses 140. The analyses are statistical analyses or application of one or more trained models to predict at least a cancer status of the individual from whom the sample is derived. Different genetic features may be evaluated and considered, such as methylation of CpG sites, single nucleotide polymorphisms (SNPs), insertions or deletions (indels), other types of genetic mutation, etc. In the context of methylation, analyses 140 may include tissue purity assessment 142 (e.g., further described in FIGS. 4A, 4B, 5A, 5B, 6, and 7), feature extraction 144, and applying a cancer classifier 146 to determine a cancer prediction (e.g., further described in FIGS. 9A & 9B). Tissue purity assessment 142 involves applying a mixture model to deconvolute proportions of tissue components contributing DNA fragments to the sample. In general, tissue purity assessment 142 may be used to determine what proportion of methylation sequence reads for a sample were shed from cancerous tissue compared to a proportion of methylation sequence reads from non-cancer cells. Tissue purity assessment 142 may be particularly useful in deconvolving heterogeneous tumors that comprise multiple clonal populations with potentially distinct genetic signatures. The mixture model may also be applied to quantify cancer signal or grade cancer status based on the determined proportions. The cancer classifier 146 inputs the extracted features to determine a cancer prediction. The cancer prediction may be a label or a value. The label may indicate a particular cancer state, e.g., binary labels can indicate presence or absence of cancer, multiclass labels can indicate one or more cancer types from a plurality of cancer types that are screened for, cancer stage, etc. The value may indicate a likelihood of a particular cancer state, e.g., a likelihood of cancer, and/or a likelihood of a particular cancer type.

The analytics system returns the prediction 150 to the healthcare provider. The prediction 150 may include binary prediction of presence or absence of cancer, a particular cancer type, cancer stage, tissue proportions, etc. The healthcare provider may establish or adjust a treatment plan based on the returned prediction 150. Optimization of treatment is further described in Section V.C. Treatment.

FIG. 1B is a flowchart of a method 160 for identifying a plurality of features for generating a classifier to predict a disease state (e.g., presence or absence of a disease, type of disease, and/or a disease tissue of origin), according to various embodiments. In some embodiments, the analytics system 200 performs the method 160 to process sequence reads of fragments from nucleic acid samples. The method 160 includes, but is not limited to, the following steps: generating 165 sequence reads; training 170 probabilistic models associated with each of a plurality of different disease states (e.g., different cancer types); applying 175 the probabilistic models to determine a value based on a probability that a sequence read originated from a sample associated with each of the plurality of disease states associated with each probabilistic model; identifying 180 features by determining a count of sequence reads having a value exceeding a threshold; generating 185 a classifier using the features, and, optionally, applying 190 the classifier to predicting disease state and/or a tissue of origin, associated with a disease state.

### II.A. Methylation Overview

According to the present description, cfDNA fragments from an individual are treated, for example by converting unmethylated cytosines to uracils, sequenced and the sequence reads compared to a reference genome to identify the methylation states at specific CpG sites within the DNA fragments. Each CpG site may be methylated or unmethylated. Identification of informative fragments, in comparison to healthy individuals, may provide insight into a subject's cancer status. In some embodiments, sequence reads or fragments are informative (e.g., informative of a presence of a disease state) when such sequence reads or fragments are determined to be anomalously methylated. As is well known in the art, DNA methylation anomalies (compared to healthy controls) can cause different effects, which may contribute to cancer. Various challenges arise in the identification of informative cfDNA fragments. First off, determining a DNA fragment to be informative can hold weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be informative. On another note, methylation of a cytosine at a CpG site can causally influence methylation at a subsequent CpG site. To encapsulate this dependency can be another challenge in itself.

Methylation can typically occur in deoxyribonucleic acid (DNA) when a hydrogen atom on the pyrimidine ring of a cytosine base is converted to a methyl group, forming 5-methylcytosine. In particular, methylation can occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Informative DNA methylation can be identified as hypermethylation or hypomethylation, both of which may be indicative of cancer status. Throughout this disclosure, hypermethylation and hypomethylation can be characterized for a DNA fragment, if the DNA fragment comprises more than a threshold number of CpG sites with more than a threshold percentage of those CpG sites being methylated or unmethylated.

The principles described herein can be equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein can be the same, and consequently the inventive concepts described herein can be applicable to those other forms of methylation.

### II.B. Exemplary Sequencer and Analytics System

FIGS. 2A&B is a flowchart of systems and devices for sequencing nucleic acid samples according to one embodiment. This illustrative flowchart includes devices such as a sequencer 270 and an analytics system 200. The sequencer 270 and the analytics system 200 may work in tandem to perform one or more steps in the processes described herein.

In various embodiments, the sequencer 270 receives an enriched nucleic acid sample 260. As shown in FIG. 2A, the sequencer 270 can include a graphical user interface 275 that enables user interactions with particular tasks (e.g., initiate sequencing or terminate sequencing) as well as one more loading stations 280 for loading a sequencing cartridge including the enriched fragment samples and/or for loading necessary buffers for performing the sequencing assays. Therefore, once a user of the sequencer 270 has provided the necessary reagents and sequencing cartridge to the loading station 280 of the sequencer 270, the user can initiate sequencing by interacting with the graphical user interface 275 of the sequencer 270. Once initiated, the sequencer 270 performs the sequencing and outputs the sequence reads of the enriched fragments from the nucleic acid sample 260.

In some embodiments, the sequencer 270 is communicatively coupled with the analytics system 200. The analytics system 200 includes some number of computing devices used for processing the sequence reads for various applications such as assessing methylation status at one or more CpG sites, variant calling or quality control. The sequencer 270 may provide the sequence reads in a BAM file format to the analytics system 200. The analytics system 200 can be communicatively coupled to the sequencer 270 through a wireless, wired, or a combination of wireless and wired communication technologies. Generally, the analytics system 200 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 200 may label a sequence read with one or more genes that align to the sequence read. In one embodiment, fragment length (or size) is determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. In one embodiment, the read pair R_1 and R_2 can be assembled into a fragment, and the fragment used for subsequent analysis and/or classification. An output file having SAM (sequence alignment map) format or BAM (binary) format may be generated and output for further analysis.

Referring now to FIG. 2B, FIG. 2B is a block diagram of an analytics system 200 for processing DNA samples according to one embodiment. The analytics system implements one or more computing devices for use in analyzing DNA samples. The analytics system 200 includes a sequence processor 210, sequence database 215, model database 225, one or more probabilistic models 230 and/or one or more classifiers 240, and parameter database 235. In some embodiments, the analytics system 200 performs one or more steps in the methods or processes disclosed herein.

The sequence processor 210 generates methylation state vectors for fragments from a sample. At each CpG site on a fragment, the sequence processor 210 generates a methylation state vector for each fragment specifying a location of the fragment in the reference genome, a number of CpG sites in the fragment, and the methylation state of each CpG site in the fragment whether methylated, unmethylated, or indeterminate via the process 360 of FIG. 4B. The sequence processor 210 may store methylation state vectors for fragments in the sequence database 215. Data in the sequence database 215 may be organized such that the methylation state vectors from a sample are associated to one another.

Further, multiple different models 230 may be stored in the model database 225 or retrieved for use with test samples. In one example, a model is a trained cancer classifier 240 for determining a cancer prediction for a test sample using a feature vector derived from informative fragments. The training and use of the cancer classifier is discussed elsewhere herein. The analytics system 200 may train the one or more models 230 and/or one or more classifiers 240 and store various trained parameters in the parameter database 235. The analytics system 200 stores the models 230 and/or classifiers along with functions in the model database 225.

During inference, the machine learning engine 220 uses the one or more models 230 and/or classifiers 240 to return outputs. The machine learning engine accesses the models 230 and/or classifiers 240 in the model database 225 along with trained parameters from the parameter database 235. According to each model, the machine learning engine 220 receives an appropriate input for the model and calculates an output based on the received input, the parameters, and a function of each model relating the input and the output. In some use cases, the machine learning engine 220 further calculates metrics correlating to a confidence in the calculated outputs from the model. In other use cases, the machine learning engine 220 calculates other intermediary values for use in the model.

### II.C. Assay Protocol

FIG. 3 is a flowchart describing a process 300 of sequencing nucleic acids, according to an embodiment. In some embodiments, the process 300 is performed to generate the sequence reads as part of step 110 of the method 100 of FIG. 1.

In step 310, a nucleic acid sample (e.g., DNA or RNA) is extracted from a subject. In the present disclosure, DNA and RNA can be used interchangeably unless otherwise indicated. That is, the embodiments described herein can be applicable to both DNA and RNA types of nucleic acid sequences. However, the examples described herein can focus on DNA for purposes of clarity and explanation. The sample can include nucleic acid molecules derived from any subset of the human genome, including the whole genome. The sample can include blood, plasma, serum, urine, fecal, saliva, other types of bodily fluids, or any combination thereof. In some embodiments, methods for drawing a blood sample (e.g., syringe or finger prick) can be less invasive than procedures for obtaining a tissue biopsy, which can require surgery. The extracted sample can comprise cfDNA and/or ctDNA. If a subject has a disease state, such as cancer, cell free nucleic acids (e.g., cfDNA) in an extracted sample from the subject generally includes detectable level of the nucleic acids that can be used to assess a disease state.

In step 315, the extracted nucleic acids (e.g., including cfDNA fragments) are treated to convert unmethylated cytosines to uracils. In some embodiments, the method 300 uses a bisulfite treatment of the samples which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, CA)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, e.g., APOBEC-Seq (NEBiolabs, Ipswich, MA).

In step 320, a sequencing library is prepared. In some embodiments, the preparation includes at least two steps. In a first step, a ssDNA adapter is added to the 3′-OH end of a bisulfite-converted ssDNA molecule using a ssDNA ligation reaction. In some embodiments, the ssDNA ligation reaction uses CircLigase II (Epicentre) to ligate the ssDNA adapter to the 3′-OH end of a bisulfite-converted ssDNA molecule, wherein the 5′-end of the adapter is phosphorylated and the bisulfite-converted ssDNA has been dephosphorylated (i.e., the 3′ end has a hydroxyl group). In another embodiment, the ssDNA ligation reaction uses Thermostable 5′ AppDNA/RNA ligase (available from New England BioLabs (Ipswich, MA)) to ligate the ssDNA adapter to the 3′-OH end of a bisulfite-converted ssDNA molecule. In this example, the first UMI adapter is adenylated at the 5′-end and blocked at the 3′-end. In another embodiment, the ssDNA ligation reaction uses a T4 RNA ligase (available from New England BioLabs) to ligate the ssDNA adapter to the 3′-OH end of a bisulfite-converted ssDNA molecule.

In a second step, a second strand DNA is synthesized in an extension reaction. For example, an extension primer, that hybridizes to a primer sequence included in the ssDNA adapter, is used in a primer extension reaction to form a double-stranded bisulfite-converted DNA molecule. Optionally, in some embodiments, the extension reaction uses an enzyme that is able to read through uracil residues in the bisulfite-converted template strand.

Optionally, in a third step, a dsDNA adapter is added to the double-stranded bisulfite-converted DNA molecule. Then, the double-stranded bisulfite-converted DNA can be amplified to add sequencing adapters. For example, PCR amplification using a forward primer that includes a P5 sequence and a reverse primer that includes a P7 sequence is used to add P5 and P7 sequences to the bisulfite-converted DNA. Optionally, during library preparation, unique molecular identifiers (UMI) can be added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In an optional step 325, the nucleic acids (e.g., fragments) can be hybridized. Hybridization probes (also referred to herein as “probes”) may be used to target, and pull down, nucleic acid fragments informative for disease states. For a given workflow, the probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand. The probes can range in length from 10s, 100s, or 1000s of base pairs. Moreover, the probes can cover overlapping portions of a target region.

In an optional step 330, the hybridized nucleic acid fragments are captured and can be enriched, e.g., amplified using PCR. In some embodiments, targeted DNA sequences can be enriched from the library. This is used, for example, where a targeted panel assay is being performed on the samples. For example, the target sequences can be enriched to obtain enriched sequences that can be subsequently sequenced. In general, any known method in the art can be used to isolate, and enrich for, probe-hybridized target nucleic acids. For example, as is well known in the art, a biotin moiety can be added to the 5′-end of the probes (i.e., biotinylated) to facilitate isolation of target nucleic acids hybridized to probes using a streptavidin-coated surface (e.g., streptavidin-coated beads).

In step 335, sequence reads are generated from the nucleic acid sample, e.g., enriched sequences. Sequencing data can be acquired from the enriched DNA sequences by known means in the art. For example, the method can include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In step 340, the sequence processor 210 can generate methylation information using the sequence reads. A methylation state vector can then be generated using the methylation information determined from the sequence reads. FIG. 4B is an illustration of the process 360, starting from process 300 of FIG. 3 of sequencing a cfDNA molecule, to obtain a methylation state vector 352, according to an embodiment. As an example, the analytics system receives a cfDNA molecule 312 that, in this example, contains three CpG sites. As shown, the first and third CpG sites of the cfDNA molecule 312 are methylated 314. During the treatment step 315, the cfDNA molecule 312 is converted to generate a converted cfDNA molecule 322. During the treatment 315, the second CpG site which was unmethylated has its cytosine converted to uracil. However, the first and third CpG sites were not converted.

After conversion, a sequencing library 330 is prepared and sequenced generating a sequence read 342. The analytics system aligns (not shown) the sequence read 342 to a reference genome 344. The reference genome 344 provides the context as to what position in a human genome the fragment cfDNA originates from. In this simplified example, the analytics system aligns the sequence read 342 such that the three CpG sites correlate to CpG sites 23, 24, and 25 (arbitrary reference identifiers used for convenience of description). The analytics system thus generates information both on methylation status of all CpG sites on the cfDNA molecule 312 and the position in the human genome that the CpG sites map to. As shown, the CpG sites on sequence read 342 which were methylated are read as cytosines. In this example, the cytosines appear in the sequence read 342 only in the first and third CpG site which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the analytics system generates 200 a methylation state vector 352 for the fragment cfDNA 312. In this example, the resulting methylation state vector 352 is <M₂₃, U₂₄, M₂₅>, wherein M corresponds to a methylated CpG site, U corresponds to an unmethylated CpG site, and the subscript number corresponds to a position of each CpG site in the reference genome.

### II.D. Identifying Informative Fragments

In some embodiments, the analytics system determines informative fragments for a sample using the sample's methylation state vectors. For example, for each nucleic acid molecule or fragment in a sample, the analytics system determines whether the nucleic acid molecule or fragment is an informative molecule or fragment (via analysis of sequence reads derived therefrom), relative to an expected methylation state vector from a healthy sample using the methylation state vector corresponding to the nucleic acid molecule. In one embodiment, the analytics system calculates a p-value score for each methylation state vector describing a probability of observing that methylation state vector or other methylation state vectors even less probable in the healthy control group (as described, for example, in U.S. Pat. Appl. Pub. No. 2019/0287652, which is incorporated herein by reference). The process for calculating a p-value score will also be discussed below in Section II.B.i. P-Value Filtering. The analytics system may determine, and optionally filter out, sequence reads of nucleic acid molecules or fragments with a methylation state vector having below a threshold p-value score as informative fragments. In another embodiment, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining informative molecules or fragments. Examples of other probabilistic models include a mixture model, a deep probabilistic model, etc. In some embodiments, the analytics system may use any combination of the processes described below for identifying informative fragments. With the identified informative fragments, the analytics system may filter the set of methylation state vectors for a sample for use in other processes, e.g., for use in training and deploying a cancer classifier.

### II.D.i. P-Value Filtering

In one embodiment, the analytics system calculates a p-value score for each methylation state vector compared to methylation state vectors from fragments in a healthy control group. The p-value score describes a probability of observing a nucleic acid molecule having the methylation status matching that methylation state vector in the healthy control group. In order to determine a DNA fragment to be informative, the analytics system uses a healthy control group with a majority of fragments that are normally methylated. When conducting this probabilistic analysis for determining informative fragments, the determination holds weight in comparison with the group of control subjects that make up the healthy control group. To ensure robustness in the healthy control group, the analytics system may select some threshold number of healthy individuals to source samples including DNA fragments. FIG. 4B below describes the method of generating a data structure for a healthy control group with which the analytics system can calculate p-value scores. FIG. 4C describes the method of calculating a p-value score with the generated data structure.

FIG. 4B is a flowchart describing a process 400 of generating a data structure for a healthy control group, according to an embodiment. To create a healthy control group data structure, the analytics system receives a plurality of DNA fragments (e.g., cfDNA) from a plurality of healthy individuals. A methylation state vector is identified for each fragment, for example via the process 360.

With each fragment's methylation state vector, the analytics system subdivides 405 the methylation state vector into strings of CpG sites. In one embodiment, the analytics system subdivides 405 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 would result in 4 strings of length 4, 5 strings of length 3, 6 strings of length 2, and 7 strings of length 1. If a methylation state vector is shorter than or the same length as the specified string length, then the methylation state vector may be converted into a single string containing all of the CpG sites of the vector.

The analytics system 200 tallies 410 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system tallies 410 how many occurrences of each methylation state vector possibility come up in the control group. Continuing this example, this may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site x in the reference genome. The analytics system creates 415 the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of 4 means that every CpG site has at the very least 2\hat{ }4 numbers to tally for strings of length 4. Increasing the maximum string length to 5 means that every CpG site has an additional 2\hat{ }4 or 16 numbers to tally, doubling the numbers to tally (and computer memory required) compared to the prior string length. Reducing string size helps keep the data structure creation and performance (e.g., use for later accessing as described below), in terms of computational and storage, reasonable. Second, a statistical consideration to limiting the maximum string length is to avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it requires a significant amount of data that may not be available, and thus would be too sparse for a model to perform appropriately. For example, calculating a probability of anomalousness/cancer conditioned on the prior 100 CpG sites would require counts of strings in the data structure of length 100, ideally some matching exactly the prior 100 methylation states. If only sparse counts of strings of length 100 are available, there will be insufficient data to determine whether a given string of length of 100 in a test sample is informative or not.

FIG. 4C is a flowchart describing a process 420 for identifying informative fragments from an individual, according to an embodiment. In process 420, the analytics system generates methylation state vectors 352 from cfDNA fragments of the subject. The analytics system handles each methylation state vector as follows.

For a given methylation state vector, the analytics system enumerates 430 all possibilities of methylation state vectors having the same starting CpG site and same length (i.e., set of CpG sites) in the methylation state vector. As each methylation state is generally either methylated or unmethylated there are effectively two possible states at each CpG site, and thus the count of distinct possibilities of methylation state vectors depends on a power of 2, such that a methylation state vector of length n would be associated with 2ⁿ possibilities of methylation state vectors. With methylation state vectors inclusive of indeterminate states for one or more CpG sites, the analytics system may enumerate 430 possibilities of methylation state vectors considering only CpG sites that have observed states.

The analytics system 200 calculates 440 the probability of observing each possibility of methylation state vector for the identified starting CpG site and methylation state vector length by accessing the healthy control group data structure. In one embodiment, calculating the probability of observing a given possibility uses a Markov chain probability to model the joint probability calculation. In other embodiments, calculation methods other than Markov chain probabilities are used to determine the probability of observing each possibility of methylation state vector.

The analytics system calculates 450 a p-value score for the methylation state vector using the calculated probabilities for each possibility. In one embodiment, this includes identifying the calculated probability corresponding to the possibility that matches the methylation state vector in question. Specifically, this is the possibility having the same set of CpG sites, or similarly the same starting CpG site and length as the methylation state vector. The analytics system sums the calculated probabilities of any possibilities having probabilities less than or equal to the identified probability to generate the p-value score.

This p-value represents the probability of observing the methylation state vector of the fragment or other methylation state vectors even less probable in the healthy control group. A low p-value score, thereby, generally corresponds to a methylation state vector which is rare in a healthy individual, and which causes the fragment to be labeled informative, relative to the healthy control group. A high p-value score generally relates to a methylation state vector is expected to be present, in a relative sense, in a healthy individual. If the healthy control group is a non-cancerous group, for example, a low p-value indicates that the fragment is informative relative to the non-cancer group, and therefore possibly indicative of the presence of cancer in the test subject.

As above, the analytics system calculates p-value scores for each of a plurality of methylation state vectors, each representing a cfDNA fragment in the test sample. To identify which of the fragments are informative, the analytics system may filter 460 the set of methylation state vectors based on their p-value scores. In one embodiment, filtering is performed by comparing the p-values scores against a threshold and keeping only those fragments below the threshold. This threshold p-value score could be on the order of 0.1, 0.01, 0.001, 0.0001, or similar.

According to example results from the process, the analytics system yields a median (range) of 2,800 (1,500-12,000) fragments with informative methylation patterns for participants without cancer in training, and a median (range) of 3,000 (1,200-220,000) fragments with informative methylation patterns for participants with cancer in training. These filtered sets of fragments with informative methylation patterns may be used for the downstream analyses as described below.

In one embodiment, the analytics system uses 455 a sliding window to determine possibilities of methylation state vectors and calculate p-values. Rather than enumerating possibilities and calculating p-values for entire methylation state vectors, the analytics system enumerates possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window identifies the sequential set of CpG sites from the vector within the window starting from the first CpG site in the vector. The analytic system calculates a p-value score for the window including the first CpG site. The analytics system then “slides” the window to the second CpG site in the vector, and calculates another p-value score for the second window. Thus, for a window size l and methylation vector length m, each methylation state vector will generate m−l+l p-value scores. After completing the p-value calculations for each portion of the vector, the lowest p-value score from all sliding windows is taken as the overall p-value score for the methylation state vector. In another embodiment, the analytics system aggregates the p-value scores for the methylation state vectors to generate an overall p-value score.

Using the sliding window helps to reduce the number of enumerated possibilities of methylation state vectors and their corresponding probability calculations that would otherwise need to be performed. To give a realistic example, it is possible for fragments to have upwards of 54 CpG sites. Instead of computing probabilities for 2\hat{ }54 (˜1.8×10\hat{ }16) possibilities to generate a single p-score, the analytics system can instead use a window of size 5 (for example) which results in 50 p-value calculations for each of the 50 windows of the methylation state vector for that fragment. Each of the 50 calculations enumerates 2\hat{ }5 (32) possibilities of methylation state vectors, which total results in 50×2\hat{ }5 (1.6×10\hat{ }3) probability calculations. This results in a vast reduction of calculations to be performed, with no meaningful hit to the accurate identification of informative fragments.

In embodiments with indeterminate states, the analytics system may calculate a p-value score summing out CpG sites with indeterminates states in a fragment's methylation state vector. The analytics system identifies all possibilities that have consensus with the all methylation states of the methylation state vector excluding the indeterminate states. The analytics system may assign the probability to the methylation state vector as a sum of the probabilities of the identified possibilities. As an example, the analytics system calculates a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states uses calculations of probabilities of possibilities up to 2\hat{ }i, wherein i denotes the number of indeterminate states in the methylation state vector. In additional embodiments, a dynamic programming algorithm may be implemented to calculate the probability of a methylation state vector with one or more indeterminate states. Advantageously, the dynamic programming algorithm operates in linear computational time.

In one embodiment, the computational burden of calculating probabilities and/or p-value scores may be further reduced by caching at least some calculations. For example, the analytic system may cache in transitory or persistent memory calculations of probabilities for possibilities of methylation state vectors (or windows thereof). If other fragments have the same CpG sites, caching the possibility probabilities allows for efficient calculation of p-score values without needing to re-calculate the underlying possibility probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

### II.D.ii Hypermethylated Fragments and Hypomethylated Fragments

In some embodiments, the analytics system determines informative fragments as fragments with over a threshold number of CpG sites and either with over a threshold percentage of the CpG sites methylated or with over a threshold percentage of CpG sites unmethylated; the analytics system identifies such fragments as hypermethylated fragments or hypomethylated fragments. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

### II.E. Blocks of Reference Genome

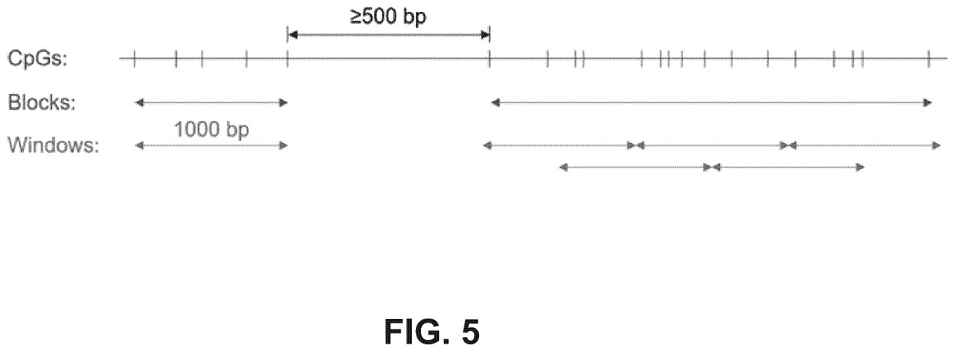

FIG. 5 is an illustration of blocks of a reference genome, according to an embodiment. The sequence processor 210 can partition a reference genome (or a subset of the reference genome) in one or more stages, e.g., for use cases involving a targeted methylation assay. For instance, the sequence processor 210 separates the reference genome into blocks of CpG sites. Each block is defined when there is a separation between two adjacent CpG sites that exceeds a threshold, e.g., greater than 200 base pairs (bp), 300 bp, 400 bp, 500 bp, 600 bp, 700 bp, 800 bp, 900 bp, or 1,000 bp, among other values. Thus, blocks can vary in size of base pairs. For each block, the sequence processor 210 can subdivide the block into windows of a certain length, e.g., 500 bp, 600 bp, 700 bp, 800 bp, 900 bp, 1,000 bp, 1,100 bp, 1,200 bp, 1,300 bp, 1,400 bp, or 1,500 bp, among other values. In other embodiments, the windows can be from 200 bp to 10 kilobase pairs (kbp), from 500 bp to 2 kbp, or about 1 kbp in length. Windows (e.g., that are adjacent) can overlap by a number of base pairs or a percentage of the length, e.g., 10%, 20%, 30%, 40%, 50%, or 60%, among other values. Windows can be separated between two adjacent CpG sites that exceeds a threshold, e.g., greater than 200 base pairs (bp), 300 bp, 400 bp, 500 bp, 600 bp, 700 bp, 800 bp, 900 bp, or 1,000 bp, among other values.

The sequence processor 210 can analyze sequence reads derived from DNA fragments using a windowing process. In particular, the sequence processor 210 scans through the blocks window-by-window and reads fragments within each window. The fragments can originate from tissue and/or high-signal cfDNA. High-signal cfDNA samples can be determined by a binary classification model, by cancer stage, or by another metric. By partitioning the reference genome (e.g., using blocks and windows), the sequence processor 210 can facilitate computational parallelization. Moreover, the sequence processor 210 can reduce computational resources to process a reference genome by targeting the sections of base pairs that include CpG sites, while skipping other sections that do not include CpG sites.

### III. MODEL BASED FEATURE ENGINEERING AND CLASSIFICATION

### III.A. Model Based Feature Engineering

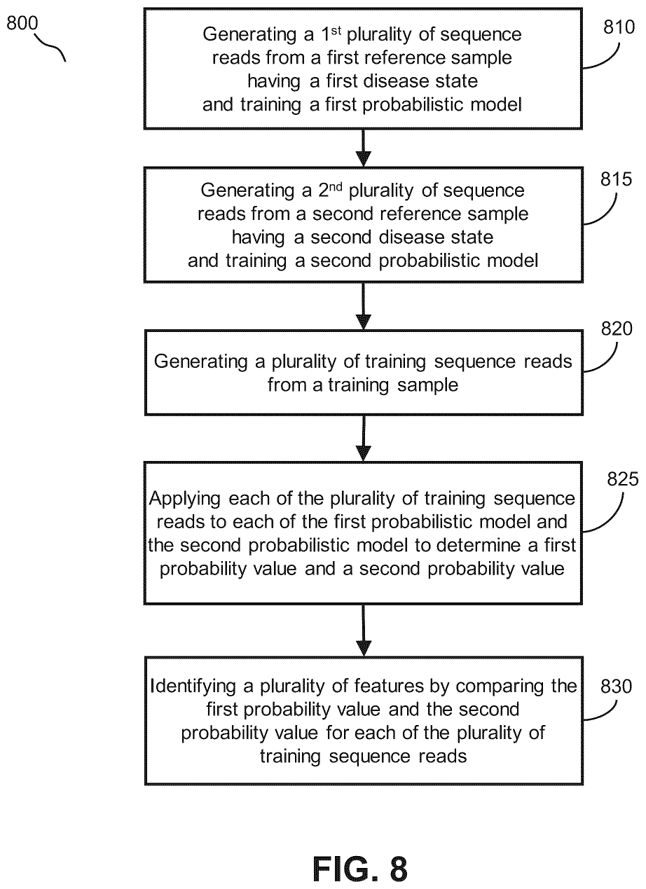

In accordance with one embodiment, as illustrated in FIG. 8, the present disclosure is directed to model-based feature engineering for deriving features useful for classification of a disease state. As described elsewhere herein, the disease state can be the presence or absence of a disease, a type of disease, and/or a disease tissue or origin. For example, as described herein, the disease state can be the presence or absence of cancer, a type of cancer, and/or a cancer tissue of origin. The type of cancer and/or cancer tissue of origin can be selected from the group including breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, such as lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia, among other types of cancer.

In step 810, a first plurality of sequence reads are generated, as described elsewhere herein, from a first reference sample having a first disease state, and a second plurality of sequence reads are generated from a second reference sample having a second disease state. The first plurality of sequence reads and/or the second plurality of sequence reads can be more than 10,000, more than 50,000, more than 100,000, more than 200,000, more than 500,000, more than 1,000,000, more than 2,000,000, more than 5,000,000, or more than 10,000,000 sequence reads. As used herein a “reference sample” is a sample obtained from a subject with a known disease state.

In some embodiments, one or more reference samples, having one or more known disease state, can be used to train one or more probabilistic models, that in turn can be used to derive features for classifying a disease state of an unknown test sample.

The sample can be a genomic DNA (gDNA) sample or a cell free DNA (cfDNA) sample. The reference sample can be a blood, plasma, serum, urine, fecal, and saliva samples. Alternatively, the reference sample can be whole blood, a blood fraction, a tissue biopsy, pleural fluid, pericardial fluid, cerebral spinal fluid, and peritoneal fluid. In some embodiments, the first reference sample is obtained from a subject known to have cancer and the second reference sample is obtained from a healthy subject or a non-cancer subject. In some embodiments, the first reference sample is obtained from a subject known to have a first type of cancer (e.g., lung cancer) and the second reference sample is obtained from a subject known to have a second type of cancer (e.g., breast cancer). In still other embodiments, the first reference sample is obtained from a subject known to have a first disease tissue of origin (e.g., lung disease) and a second reference sample is obtained from a second disease state tissue of origin (e.g., a liver disease).

In step 815, the machine learning engine 220 trains a first probabilistic model 230 and a second probabilistic model 230, from the first plurality of sequence reads and the second plurality of sequence reads (generated in step 110), respectively, each probabilistic model associated with a different disease state of one or more possible disease states. As previously described, the disease state can be the presence or absence of cancer, a type of cancer, and/or a cancer tissue of origin. In various embodiments, training data is split into K subsets (folds) for K-fold cross-validation. Folds can be balanced for: cancer/non-cancer status, tissue of origin, cancer stage, age (e.g., grouped in 10-year buckets), gender, ethnicity, and smoking status, among other factors. Data from K−1 of the folds may be used as training data for the probabilistic models, and the held-out fold may be used as testing data.

The machine learning engine 220 trains the first and second probabilistic models 230, for the first and second disease states, respectively, by fitting each of the probabilistic models 230 to the first plurality and second plurality of sequence reads, respectively. For example, in one embodiment, the first probabilistic model is fitted using a first plurality of sequence reads derived from one or more samples from subjects known to have cancer and the second probabilistic model is fitted using the second plurality of sequence reads derived from one or more samples from healthy subjects or non-cancer subjects. In other embodiments, the first probabilistic model can be trained for a first type of cancer or a first tissue of origin and the second probabilistic model can be trained for a second type of cancer or a second tissue of origin. As one of skill in the art would appreciate, any number of disease state probabilistic models can be trained utilizing sequence reads derived from one or more sample taken from subjects with any one of a number of possible disease states. For example, in some embodiments, additional cancer-specific probabilistic models (i.e., for additional types of cancer and or tissues of origin models) can be trained for a third, fourth, fifth, sixth, seventh, eighth, ninth, tenth, etc. (e.g., up to twenty, thirty, or more) specific type of cancer and used to determine probabilities that sequence reads from a training set, or an unknown cancer type, are more likely derived from one cancer type (or cancer tissue of origin) than another cancer type (or cancer tissue of origin), as described elsewhere herein.

As used herein a “probabilistic model” is any mathematical model capable of assigning a probability to a sequence read based on methylation status at one or more sites on the read. During training, the machine learning engine 220 fits sequence reads derived from one or more samples from subjects having a known disease and can be used to determine sequence reads probabilities indicative of a disease state utilizing methylation information or methylation state vectors (e.g., previously described with respect to FIGS. 3-4). In particular, in one embodiment, the machine learning engine 220 determines observed rates of methylation for each CpG site within a sequence read. The rate of methylation represents a fraction or percentage of base pairs that are methylated within a CpG site. The trained probabilistic model 230 can be parameterized by products of the rates of methylation. In general, any known probabilistic model for assigning probabilities to sequence reads from a sample can be used. For example, the probabilistic model can be a binomial model, in which every site (e.g., CpG site) on a nucleic acid fragment is assigned a probability of methylation, or an independent sites model, in which each CpG's methylation is specified by a distinct methylation probability with methylation at one site assumed to be independent of methylation at one or more other sites on the nucleic acid fragment.

In some embodiments, the probabilistic model 230 is a Markov model, in which the probability of methylation at each CpG site is dependent on the methylation state at some number of preceding CpG sites in the sequence read, or nucleic acid molecule from which the sequence read is derived. See, e.g., U.S. patent application Ser. No. 16/352,602, entitled “Anomalous Fragment Detection and Classification,” and filed Mar. 13, 2019.

In some embodiments, the probabilistic model 230 is a “mixture model” fitted using a mixture of components from underlying models. For example, in some embodiments, the mixture components can be determined using multiple independent sites models, where methylation (e.g., rates of methylation) at each CpG site is assumed to be independent of methylation at other CpG sites. Utilizing an independent sites model, the probability assigned to a sequence read, or the nucleic acid molecule from which it derives, is the product of the methylation probability at each CpG site where the sequence read is methylated and one minus the methylation probability at each CpG site where the sequence read is unmethylated. In accordance with this embodiment, the machine learning engine 220 determines rates of methylation of each of the mixture components. The mixture model is parameterized by a sum of the mixture components each associated with a product of the rates of methylation. A probabilistic model Pr of n mixture components can be represented as:

$$Pr ( fragment \vert \{ {β}_{ki} , {f}_{k} \} ) = {\sum }_{k = 1}^{n} {f}_{k} {\prod }_{i} {{β}_{ki}^{{m}_{i}} ( 1 - {β}_{ki} )}^{1 - {m}_{i}}$$

For an input fragment, mᵢ∈{0, 1} represents the fragment's observed methylation status at position i of a reference genome, with 0 indicating unmethylation and 1 indicating methylation. A fractional assignment to each mixture component k is fₖ, where fₖ≥0 and Σₖ₌₁ⁿ fₖ=1. The probability of methylation at position i in a CpG site of mixture component k is βₖᵢ. Thus, the probability of unmethylation is 1−βₖᵢ. The number of mixture components n can be 1, 2, 3, 4, 5, 6, 7, 8, 9, 10, etc.

In some embodiments, the machine learning engine 220 fits the probabilistic model 230 using maximum-likelihood estimation to identify a set of parameters {βₖᵢ, fₖ} that maximizes the log-likelihood of all fragments deriving from a disease state, subject to a regularization penalty applied to each methylation probability with regularization strength r. The maximized quantity for N total fragments can be represented as:

$${\sum }_{j}^{N} ln ( Pr ( {fragment}_{j} \vert \{ {β}_{ki} , {f}_{k} \} ) ) + r \cdot ln ( {β}_{ki} ( 1 - {β}_{ki} ) )$$

As one of skill in the art would appreciate, other means can be used to fit the probabilistic models or to identify parameters that maximize the log-likelihood of all sequence reads derived from the reference samples. For example, in one embodiment, Bayesian fitting (using e.g., Markov chain Monte Carlo), in which each parameter is not assigned a single value but instead is associated to a distribution, is used. In other embodiments, gradient-based optimization, in which the gradient of the likelihood (or log-likelihood) with respect to the parameter values is used to step through parameter space towards an optimum, is used. In other embodiments, expectation-maximization, in which a set of latent parameters (such as identities of the mixture component from which each fragment is derived) are set to their expected values under the previous model parameters, and then the model's parameters are assigned to maximize the likelihood conditional on the assumed values of those latent variables. The two-step process is then repeated until convergence.

At step 820, a plurality of training sequence reads are generated from a training sample. The plurality of training sequence reads can be more than 10,000, more than 50,000, more than 100,000, more than 200,000, more than 500,000, more than 1,000,000, more than 2,000,000, more than 5,000,000, or more than 10,000,000 sequence reads. As used herein, a “training sample” is a sample obtained from a known disease state that can be used to generate sequence reads, which are then applied to the first and/or second probability models to generate features that can be utilized for disease state classification. In step 825, the analytics system 200 applies the first and second probabilistic models 230 to determine a first probability value and a second probability value for each sequence read of the plurality of training sequence reads. The first and second probability values are determined based on a probability that the sequence read originated from a sample associated with the first disease state, and the second disease state, respectively. The analytics system 200 can repeat step 130 for any additional probabilistic models 230 (e.g., trained from sequence reads from a third, fourth, fifth, etc. reference sample) (not shown).

At step 830 one or more features are identified by comparing the first probability value and the second probability value for each of the plurality of training sequence reads. In general, a wide array of methods can be utilized to compare the first and second probability values and identify features. For example, in one embodiment, the one or more features comprise a count of outlier sequence reads of the plurality of training sequence reads where the first probability value is greater than the second probability value. The count can be a binary count, a total count of outlier sequence reads, or a total count of anonymously methylated sequence reads. In another embodiment, the one or more features comprises a count of sequence reads or fragments including a particular methylation pattern. For example, the one or more features can be a count of sequence reads or fragments that are fully methylated at each CpG site, a count of sequence reads or fragments that are partially methylated (e.g., at least 20%, 30%, 40%, 50%, 60%, 70%, 80%, 90%, or 95% methylated). In another embodiment, the one or more features are identified using an output of a discriminative classifier trained within a single genomic region (e.g., the discriminative classifier can be a multilayer perceptron or a convolutional neural net model). In another embodiment, comparing the first probability value and the second probability value comprises determining a ratio of the first probability value and the second probability value, and the one or more features comprise sequence read counts of sequence reads that exceed a ratio threshold value.

In another embodiment, the first probability value or the second probability value is a log-likelihood value. For example, the analytics system 200 can calculate a log-likelihood ratio R with the fitted probabilistic models associated with the first and second disease states, respectively. Specifically, the log-likelihood ratio can be calculated using the probabilities Pr of observing a methylation pattern on the fragment for samples associated with the first disease state and second disease state:

$${R}_{disease state} ( fragment ) ≡ ln ( \frac{Pr ( fragment \vert first disease state )}{Pr ( fragment \vert second disease state )} )$$

The analytics system 200 can identify features using multiple tiers of threshold values. For example, the tiers include threshold values of 1, 2, 3, 4, 5, 6, 7, 8, and 9. In some embodiments, a smoothing function may be applied. For example, responsive to determining that R is (e.g., significantly) less than a tier value, the analytics system 200 assigns a feature value of ˜0; responsive to determining that R equals a tier value, the analytics system 200 assigns a feature value of 0.5; responsive to determining that R is (e.g., significantly) greater than a tier value, the analytics system 200 assigns a feature value of ˜1. Each tier indicates a varying threshold that a fragment (from which the sequence reads were generated) more likely originated from a sample associated with a disease state than from a healthy sample. The analytics system 200 can use the threshold value to determine counts of outlier fragments, which can be used as features.

By filtering with a threshold value, the analytics system 200 can consider certain fragments as outliers because the fragments are unlikely to be present in healthy samples. Accordingly, outlier fragments can be considered to be more likely associated with (e.g., originating from) a disease state or a cancer sample. The number of features can vary between different tiers, e.g., one tier may have a different number of features than another tier based on the corresponding threshold values. In other embodiments, the analytics system 200 uses a different number of tiers or other threshold values. Other means for identifying features, or ranking the identified features based on measures of the features in distinguishing between different disease states (e.g., using mutual information to determine the measure of information content of a feature in distinguishing between two disease states) are described elsewhere herein.

In other embodiments, the analytics system 200 can identify a plurality of features using a different type of ratio or equation. The machine learning engine 220 can determine a fragment to be indicative of a disease state (e.g., cancer) based on whether at least one of the log-likelihood ratios considered against the various disease state is above a threshold value.

Subsequently, as described in further detail elsewhere herein, the plurality of features can be used to train a disease state classifier. For example, in some embodiments, the plurality of features can be used to train a classifier for classification of the presence or absence of cancer, a type of cancer, and/or a cancer tissue of origin.

### III.B. Disease State Tissue of Origin Classification

In accordance with another embodiment, the machine learning engine 220 trains probabilistic models 230 each associated with a different disease state of a set of multiple disease states.

The machine learning engine 220 trains probabilistic models 230 using one or more sets of sequence reads, wherein each of the one or more sets of sequence reads are generated from a different disease state of the set of multiple disease states. The disease states can include any number of types of cancer or cancer tissues of origin selected from the group including breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, such as lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia, among other types of cancer.

The machine learning engine 220 trains a probabilistic model 230, for each of the plurality of disease states, by fitting the probabilistic model 230 to the sequence reads deriving from each sample corresponding to each of the disease states. For example, in some embodiments, probabilistic models can be trained for specific types of cancer. In accordance with this embodiment, cancer-specific probabilistic models can be trained for a first, second, third, etc. specific type of cancer and used to assess a cancer type (e.g., of an unknown test sample). For example, a lung cancer-specific probabilistic model is fitted using a set of sequence reads deriving from one or more samples associated with lung cancer. As another example, a breast cancer-specific probabilistic model is fitted using a set of sequence reads deriving from one or more samples associated with breast cancer. In some embodiments, tissue specific probability models can be trained for a first, second, third, etc. tissue type and used to assess a disease state tissue of origin. For example, a first tissue of origin probabilistic model can be fitted using a set of sequence reads derived from a first tissue type (e.g., from a lung tissue sample, such as a lung biopsy) and a second tissue of origin probabilistic model can be fitted using a set of sequence reads derived from a second tissue type (e.g., from a liver tissue sample, such as a liver biopsy). Alternatively, in some embodiments, a cancer probabilistic model is fitted using a set of sequence reads derived from one or more samples from subjects known to have cancer and a non-cancer specific probabilistic model is fitted using a set of sequence reads derived from one or more samples from healthy subjects or non-cancer subjects. As one of skill in the art would appreciate, any number of disease state probabilistic models can be trained utilizing sequence reads derived from one or more sample taken from subjects with any one of a number of possible disease states. For example, in some embodiments, a plurality of sequence reads can be generated from a 3, 4, 5, 6, 7, 8, 9, 10, or more reference sample, each obtained from one or more subjects having a different disease state (e.g., different types of cancer), and used to train 3, 4, 5, 6, 7, 8, 9, 10, or more probabilistic models.

During training, the machine learning engine 220 can be trained on sequence reads indicative of a disease state utilizing methylation information or methylation state vectors (e.g., previously described with respect to FIGS. 3-4). In particular, the machine learning engine 220 determines observed rates of methylation for each CpG site within a sequence read. The rate of methylation represents a fraction or percentage of base pairs that are methylated within a CpG site. The trained probabilistic model 230 can be parameterized by products of the rates of methylation. As previously described, any known probabilistic model for assigning probabilities to sequence reads from a sample can be used. For example, the probabilistic model can be a binomial model, in which every site (e.g., CpG site) on a nucleic acid fragment is assigned a probability of methylation, or an independent sites model, in which each CpG's methylation is specified by a distinct methylation probability with methylation at one site assumed to be independent of methylation at one or more other sites on the nucleic acid fragment.

In some embodiments, a Markov model, in which the probability of methylation at each CpG site is dependent on the methylation state at some number of preceding CpG sites in the sequence read, or nucleic acid molecule from which the sequence read is derived. See, e.g., U.S. patent application Ser. No. 16/352,602, entitled “Anomalous Fragment Detection and Classification,” and filed Mar. 13, 2019.

In some embodiments, the probabilistic model 230 is a “mixture model” fitted using a mixture of components from underlying models. For example, in some embodiments, the mixture components can be determined using multiple independent sites models, where methylation (e.g., rates of methylation) at each CpG site is assumed to be independent of methylation at other CpG sites. Utilizing an independent sites model, the probability assigned to a sequence read, or the nucleic acid molecule from which it derives, is the product of the methylation probability at each CpG site where the sequence read is methylated and one minus the methylation probability at each CpG site where the sequence read is unmethylated. In accordance with this embodiment, the machine learning engine 220 determines rates of methylation of each of the mixture components. The mixture model is parameterized by a sum of the mixture components each associated with a product of the rates of methylation. A probabilistic model Pr of n mixture components can be represented as:

$$Pr ( fragment \vert \{ {β}_{ki} , {f}_{k} \} ) = {\sum }_{k = 1}^{n} {f}_{k} {\prod }_{i} {{β}_{ki}^{{m}_{i}} ( 1 - {β}_{ki} )}^{1 - {m}_{i}}$$

For an input fragment, mᵢ∈{0, 1} represents the fragment's observed methylation status at position i of a reference genome, with 0 indicating unmethylation and 1 indicating methylation. A fractional assignment to each mixture component k is fₖ, where fₖ≥0 and Σₖ₌₁ⁿ fₖ=1. The probability of methylation at position i in a CpG site of mixture component k is βₖᵢ. Thus, the probability of unmethylation is 1−βₖᵢ. The number of mixture components n can be 1, 2, 3, 4, 5, 6, 7, 8, 9, 10, etc.

In some embodiments, the machine learning engine 220 fits the probabilistic model 230 using maximum-likelihood estimation to identify a set of parameters {βₖᵢ, fₖ} that maximizes the log-likelihood of all fragments deriving from a disease state, subject to a regularization penalty applied to each methylation probability with regularization strength r. The maximized quantity for N total fragments can be represented as:

$${\sum }_{j}^{N} ln ( Pr ( {fragment}_{j} \vert \{ {β}_{ki} , {f}_{k} \} ) ) + r \cdot ln ( {β}_{ki} ( 1 - {β}_{ki} ) )$$

In step 130, the analytics system 200 applies a probabilistic model 230 to calculate values for each sequence read of a second set of sequence reads, e.g., different than the first set of sequence reads generated in step 110. The values are calculated based at least on a probability that the sequence read (and corresponding fragment) originated from a sample associated with the disease state of the probabilistic model 230. The analytics system 200 can repeat step 130 for each of the different probabilistic models 230. In some embodiments, the analytics system 200 calculates the value using a log-likelihood ratio R with the fitted probabilistic models associated with certain disease states. Specifically, the log-likelihood ratio can be calculated using the probabilities Pr of observing a methylation pattern on the fragment for samples associated with the disease state and healthy samples:

$${R}_{disease state} ( fragment ) ≡ ln ( \frac{Pr ( fragment \vert disease state )}{Pr ( fragment \vert healthy )} )$$

In other embodiments, the analytics system 200 can calculate the value using a different type of ratio or equation. The machine learning engine 220 can determine a fragment to be indicative of a disease state (e.g., cancer) based on whether at least one of the log-likelihood ratios considered against the various disease state is above a threshold value.

### III.C. Feature Selection

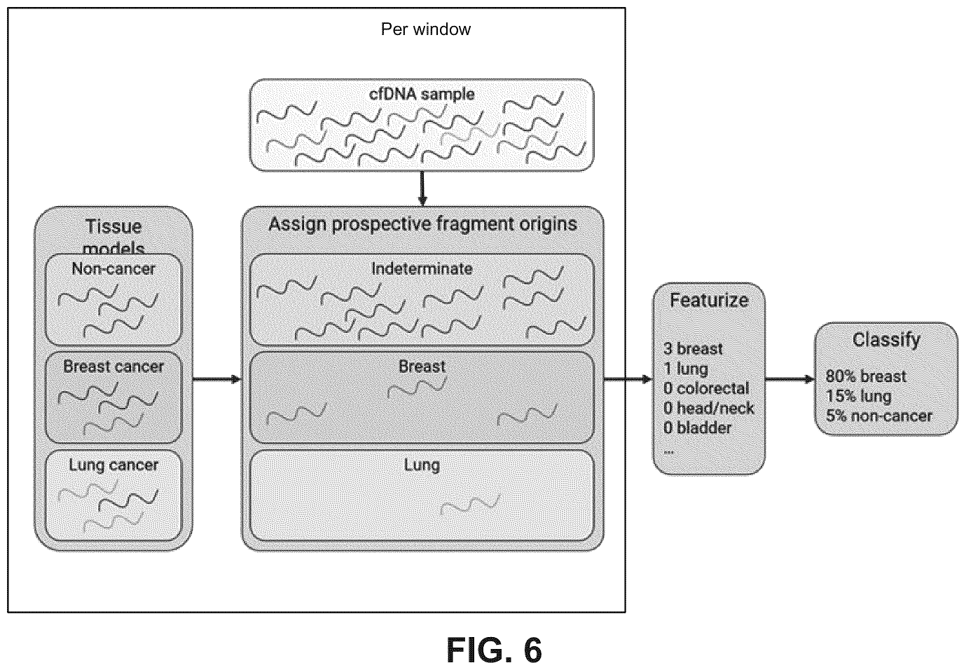

FIG. 6 is an illustration of a process of determining features to train a classifier, according to an embodiment. As previously described, the machine learning engine 220 trains probabilistic models 230 associated with disease states. In the example shown in FIG. 6, the probabilistic models 230 (“tissue models”) are associated with non-cancer (healthy), breast cancer, and lung cancer. The analytics system 200 processes one or more cfDNA and/or tumor samples to obtain fragments and uses the probabilistic models 230 to assign a value to the fragments associated with non-cancer (healthy), breast cancer, and lung cancer. The analytics system 200 can use information from sequence reads from the cfDNA and/or tumor samples to identify features for a classifier. In some embodiments, the analytics system 200 can obtain and assign fragments from each window of a partitioned referenced genome, as shown in FIG. 5. The analytics system 200 aggregates the fragments from the windows to sequence for determining features for the classifier.

The analytics system 200 identifies features by determining a count of the sequence reads having a value exceeding a threshold value. In embodiments where the value is based on the log-likelihood ratio R, the threshold value is a threshold ratio. The analytics system 200 can identify features using multiple tiers of threshold values. For example, the tiers include threshold values of 1, 2, 3, 4, 5, 6, 7, 8, and 9. Each tier indicates a varying threshold that a fragment (from which the sequence reads were generated) more likely originated from a sample associated with a disease state than from a healthy sample. The analytics system 200 can use the threshold value to determine counts of outlier fragments, which can be used as features.

By filtering with a threshold value, the analytics system 200 can consider certain fragments as outliers because the fragments are unlikely to be present in healthy samples. Accordingly, outlier fragments can be considered to be more likely associated with (e.g., originating from) a disease state or a cancer sample. The number of features can vary between different tiers. In other embodiments, the analytics system 200 uses a different number of tiers or other threshold values. In other embodiments, the analytics system 200 can filter fragments using other methods or scoring such as p-values. In some embodiments, the analytics system 200 calculates a p-value for a methylation state vector describing a probability of observing that methylation state vector or other methylation state vectors even less probable in a healthy control group. To determine a fragment to be informative, the analytics system 200 uses a healthy control group with a majority of fragments that are normally methylated (see, e.g., U.S. patent application Ser. No. 16/352,602, entitled “Anomalous Fragment Detection and Classification,” and filed Mar. 13, 2019).

The analytics system 200 can repeat steps of identifying features for each probabilistic model. As a result, the analytics system 200 can identify features for one or more disease states associated with the probabilistic models. In the example shown in FIG. 6, the analytics system 200 identifies one or more features for breast cancer and lung cancer.

In some embodiments, the analytics system 200 ranks the identified features based on measures of the features in distinguishing between different disease states. For instance, a feature is informative if the feature can distinguish a certain type of cancer from other types of cancer or healthy samples. The analytics system 200 can use mutual information to determine the measure of information content of a feature in distinguishing between two disease states. For each pair of distinct disease states, the analytics system 200 can designate one disease state, e.g., cancer type A, as a positive type and the other disease state, e.g., cancer type B, as a negative type.

The mutual information can be calculated using the estimated fraction of samples of the positive type and negative type (e.g., cancer types A and B) for which the feature is expected to be nonzero in a resulting assay. For instance, if a feature occurs frequently in healthy cfDNA, the analytics system 200 determines the feature is unlikely to occur frequently in cfDNA associated with various types of cancer. Consequently, the feature can be a weak measure in distinguishing between disease states. In calculating mutual information I, the variable Xis a certain feature (e.g., binary) and variable Y represents a disease state, e.g., cancer type A or B:

$$I ( X ; Y ) = {\sum }_{y ∈ Y} {\sum }_{x ∈ X} p ( x , y ) log log ( \frac{p ( x , y )}{p ( x ) p ( y )} )$$

$$I \approx \frac{1}{2} ( p ( 1 \vert A ) \cdot log ( \frac{p ( 1 \vert A )}{\frac{1}{2} ( p ( 1 \vert A ) + p ( 1 \vert B ) )} ) + p ( 1 \vert B ) \cdot log ( \frac{p ( 1 \vert B )}{\frac{1}{2} ( p ( 1 \vert A ) p ( 1 \vert B ) )} ) )$$

$$p ( 1 \vert A ) = {f}_{A} + {f}_{H} - {f}_{H} {f}_{A}$$

The joint probability mass function of X and Y is p (x, y) and the marginal probability mass functions are p (x) and p(y). The analytics system 200 can assume that feature absence is uninformative and either disease state is equally likely a priori, for example, p(Y=A)=p(Y=B)=0.5. The probability of observing (e.g., in cfDNA) a given binary feature of cancer type A is represented by p (1|A), where f_{A} is the probability of observing the feature in ctDNA samples from tumor (or high-signal cfDNA samples) associated with cancer type A, and f_{H} is the probability of observing the feature in a healthy or non-cancer cfDNA sample.

In some embodiments, the value of f_{A} is estimated by the fraction of cancer patients whose cfDNA would be expected to include a non-zero feature value. When the training data for cancer type A consists of cfDNA samples, this fraction can be estimated as simply the fraction of the cfDNA samples in which the feature is observed. When the training data includes tumor samples, a correction may be applied to account for the lower fraction of tumor-derived fragments in cfDNA compared to a tumor. For N fragments in a tumor sample determined to have a value greater than a threshold value (e.g., from step 140), the analytics system 200 calculates a chance r of detecting each of those fragments in cfDNA from that patient as:

$$r = \frac{cf DNA sequencing depth \times cf DNA tumor fraction}{tumor sequencing depth}$$

The probability of observing at least one fragment in cfDNA from that patient may then be calculated as, p(N_{cfDNA}>0)=1−(1−r)^{N}. To estimate f_{A}, p(N_{cfDNA}>0) may be averaged across all training samples of cancer type A, where that probability is assigned as 1 for cfDNA samples that have the feature, 0 for cfDNA samples that lack the feature, and 1−(1−r)^{N} for tumor samples. In some embodiments, the estimates are based on predetermined assumed values for tumor fraction in the cfDNA of an early-stage cancer patient (e.g., 0.1%), cfDNA sequencing depth in the final assay to be applied to patients (e.g., 1000×), and the tumor sequencing depth (e.g., 25×). To estimate f_{H}, the analytics system 200 uses a fraction of positive samples to determine how many additional samples would result in a positive detection classification at greater sequencing depth.

### III.D. Classification

The analytics system 200 generates a classifier using the features. The classifier is trained to predict, for an input sequence read from a test sample of a test subject, a tissue of origin associated with a disease state. The analytics system 200 can select a predetermined number (e.g., 128, 256, 512, 1024) of top ranking features for each pair of disease states for training the classifier, e.g., based on the mutual information calculations or another calculated measure. The predetermined number may be treated as a hyperparameter selected based on performance in cross-validation. The analytics system 200 can also select features from regions of a reference genome determined to be more informative in distinguishing between the pair of disease states. In various embodiments, the analytics system 200 keeps the best performing tier for each region and for each cancer type pair (including non-cancer as a negative type).

In some embodiments, the analytics system 200 trains the classifier by inputting sets of training samples with their feature vectors into the classifier and adjusting classification parameters so that a function of the classifier accurately relates the training feature vectors to their corresponding label. The analytics system 200 can group the training samples into sets of one or more training samples for iterative batch training of the classifier. After inputting all sets of training samples including their training feature vectors and adjusting the classification parameters, the classifier can be sufficiently trained to label test samples according to their feature vector within some margin of error. The analytics system 200 can train the classifier according to any one of a number of methods, for example, L1-regularized logistic regression or L2-regularized logistic regression (e.g., with a log-loss function), generalized linear model (GLM), random forest, multinomial logistic regression, multilayer perceptron, support vector machine, neural net, or any other suitable machine learning technique.

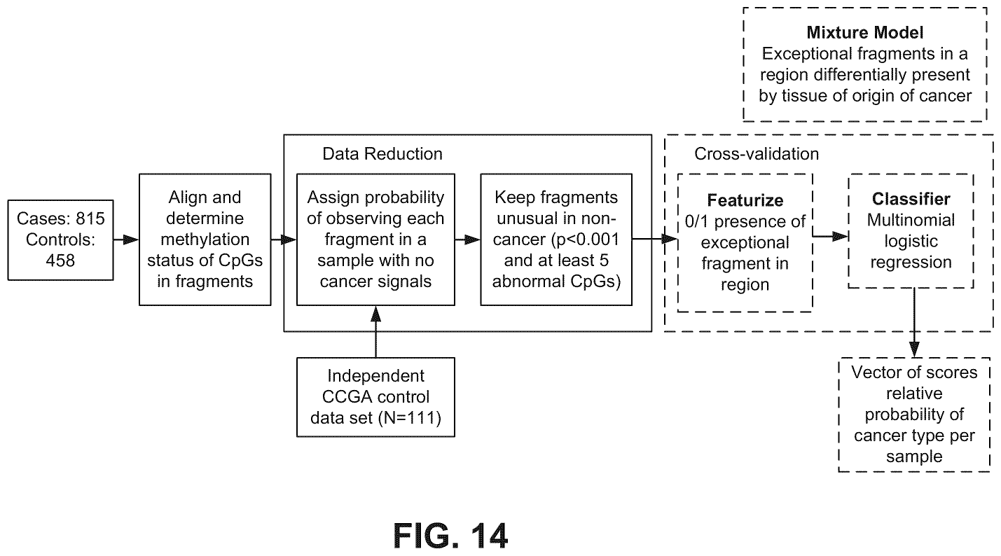

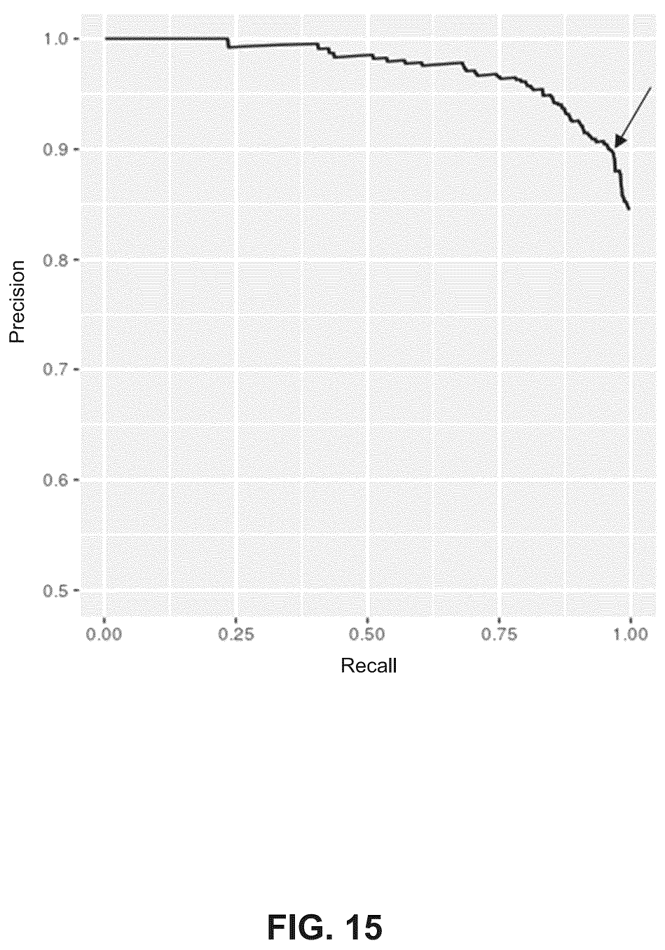

In various embodiments, the analytics system 200 transforms feature values by binarization. In particular, feature values greater than 0 are set to 1, such that feature values are either 0 or 1 (indicating presence or absence of a disease state). In other embodiments, a smoothing function may be implemented (e.g., to provide more granular values) instead of binarization to 0 or 1. As shown in FIG. 14, the analytics system 200 can binarize features in cross-validation before training a classifier with the features.

In various embodiments, the analytics system 200 trains a multinomial logistic regression classifier on the training data for a fold and generates predictions for the held-out data. For each of the K folds, the analytics system 200 trains one logistic regression for each combination of hyperparameters. An example hyperparameter is the L2 penalty, i.e., a form of regularization applied to the weights of the logistic regression. Another example hyperparameter is the topK, i.e., the number of high-ranking regions to keep for each tissue type pair (including non-cancer). For instance, where topK=16, the analytics system 200 keeps the top 16 regions per tissue type pair, as ranked by the mutual information procedure described herein. By following this procedure, the analytics system 200 can generate a prediction for each sample in the training set while ensuring that classifiers are not trained on the data for which predictions are generated.

In various embodiments, for each set of hyperparameters, the analytics system 200 evaluates performance on the cross-validated predictions of the full training set, and the analytics system 200 selects the set of hyperparameters with the best performance for retraining on the full training set. Performance may be determined based on a log-loss metric. The analytics system 200 can calculate log-loss by taking the negative logarithm of the prediction for the correct label for each sample, and then summing over samples. For instance, a perfect prediction of 1.0 for the correct label would result in a log-loss of 0 (lower is more accurate). To generate predictions for a new sample, the analytics system 200 can calculate feature values using the method described above, but restricted to features (region/positive class combinations) selected under the chosen topK value. The analytics system 200 can use the generated features to create a prediction using the trained logistic regression model.

During deployment, the analytics system 200 applies the classifier to predict a tissue of origin of a test sample, where the tissue of origin is associated with one of the disease states. In some embodiments, the classifier can return a prediction or likelihood for more than one disease state or tissue of origin. For example, the classifier can return a prediction that a test sample has a 65% likelihood of having a breast cancer tissue of origin, a 25% likelihood of having a lung cancer tissue of origin, and a 10% likelihood of having a healthy tissue of origin. The analytics system 200 can further process the prediction values to generate a single disease state determination.

### III.E. Indeterminate Localization

In various embodiments, tumor fraction can be a covariate of predictions made by a trained classifier or model across samples. As tumor fraction decreases, score assignments (e.g., based on the previously described log-likelihood ratio R) may become less definitive until the limit of classification detection is reached (i.e., probability of detection of cancer/cancer type is 50%). Samples with high cfDNA tumor fraction tend to be definitively classified, whereas samples with low cfDNA tumor fraction tend to be more ambiguous. In instances with ambiguous signal, assignments become less reliable and may be correct or incorrect by chance. In the use case of a single localization, the analytics system 200 can identify ambiguous signals and isolate those predictions to an “indeterminate localization class.”

For example, in some embodiments, the analytics system 200 can determine post-hoc indeterminate assignments from a set of tissue of origin localization vectors for individuals who have cancer scores greater than a specificity target threshold. The analytics system 200 may determine indeterminate assignments under cross validation. For each sample, the analytics system 200 can compute a metric to capture the uncertainty in the localization for that sample. As one example approach, the analytics system 200 calculates the metric using the information entropy (bits) of the tissue of origin localization, where a bit value of zero occurs when one prediction is certain. In the most ambiguous case (equal probability on all n classes), the analytics system 200 calculates a bit value of (n). As another example approach, the analytics system 200 determines the metric using the difference (delta value) between the top-ranking score and second top ranking score. A delta value of 1 occurs when one prediction is certain. A delta value of 0 occurs in the most ambiguous case. By including an indeterminate outcome, the analytics system 200 can filter out weak calls that are correct only by chance and improve the precision (e.g., fraction correct for tissue of origin assignment) for definite localization calls.

As an alternative to post-hoc indeterminate assignments, the analytics system 200 can use expectation-maximization during training to determine assignment to an indeterminate class. The analytics system 200 can also add a second layer to the classifier output to classify cases into the indeterminate class.

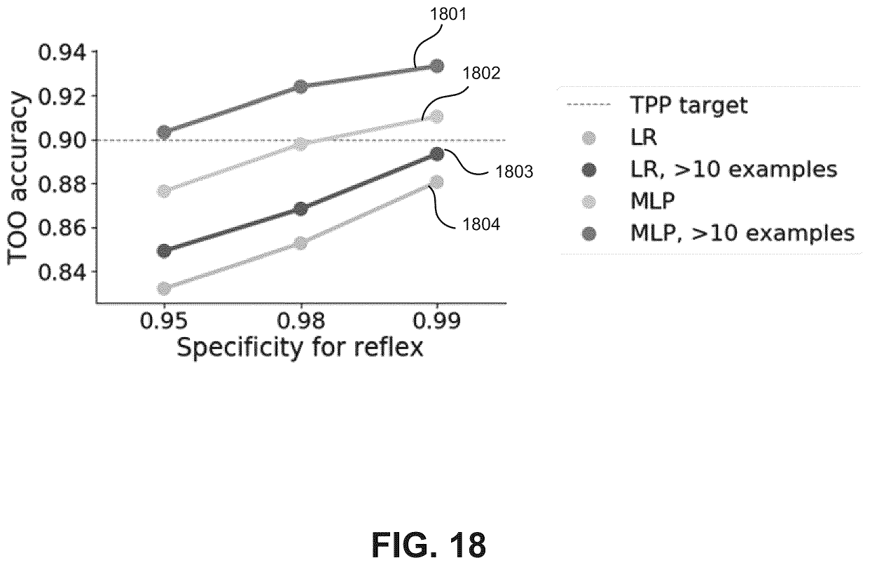

Given the metric and a record of whether each sample was correctly localized, the analytics system 200 can compute a precision-recall curve for indeterminate call thresholds, as shown FIG. 18. A cut-off point may be selected, for instance, based on a target precision level such as 90% in the example shown in FIG. 18. The analytics system 200 can compute cut-off points for localization labels individually (e.g., for a certain cancer type), or for all cancer types as a whole. Tradeoffs are subject to optimization and may depend on the cost of a wrong localization call versus the number of calls assigned an indeterminate result (e.g., precision and recall).

### III.F. Guarding Against Class Imbalance

In various embodiments, the elements score vector for an individual sample si includes posterior probabilities of the signal localization for each prediction class (e.g., disease state). Each element is scaled by the prior probability proportional to the proportion of training examples for each class:

$$p ( {c}_{i} \vert {D}_{i} ) = \frac{p ( {c}_{i} ) p ( {c}_{j} )}{p ( {D}_{i} )}$$

$$p ( {c}_{j} ) = \frac{{n}_{j}}{{\sum }_{j} {n}_{j}}$$

If the classes are imbalanced, samples with weak signal may be shifted to an inappropriate class. For example, a training set may include 99% of samples with liver cancer detections but few detections of a different cancer type. As a result, a classifier trained on this set may be skewed toward liver cancer predictions (or always guess that class). Moreover, if class proportions in classifier training are incompatible with the population frequencies (e.g., where class proportions are more balanced) to which the classifier is applied, incorrect predictions may be produced.

To assess the ability of classifiers to localize cfDNA samples from methylation and/or genomic and/or clinical features, the analytics system 200 can target proportion equivalence across classes. The analytics system 200 can calibrate scores to the incidence of disease states in a screening population optionally accounting for the detectability of the disease through tumor fraction. By modifying the prior applied to a classifier trained using a general training set, the analytics system 200 can customize the classifier to improve predictions for a specific population associated with the prior (e.g., indicating distribution of disease states in that specific population). Different geographical regions or countries may have different priors based on prevalence of specific disease states or types of cancers in the corresponding sub-population of individuals.

As an example approach, the analytics system 200 performs post-hoc recalibration of model scores. Specifically, the analytics system 200 corrects scores for a class by dividing the assigned probability by the frequency of the training set examples for that class. The correction can be optionally stabilized by adding a pseudo count. The analytics system 200 can then normalize each score vector si to sum to one.

As another approach, the analytics system 200 can re-sample low frequency training examples to the desired proportion. As yet another approach, the analytics system 200 can re-weight the loss function in classifier training.

### III.G. Conditional Cancer Signal Origin

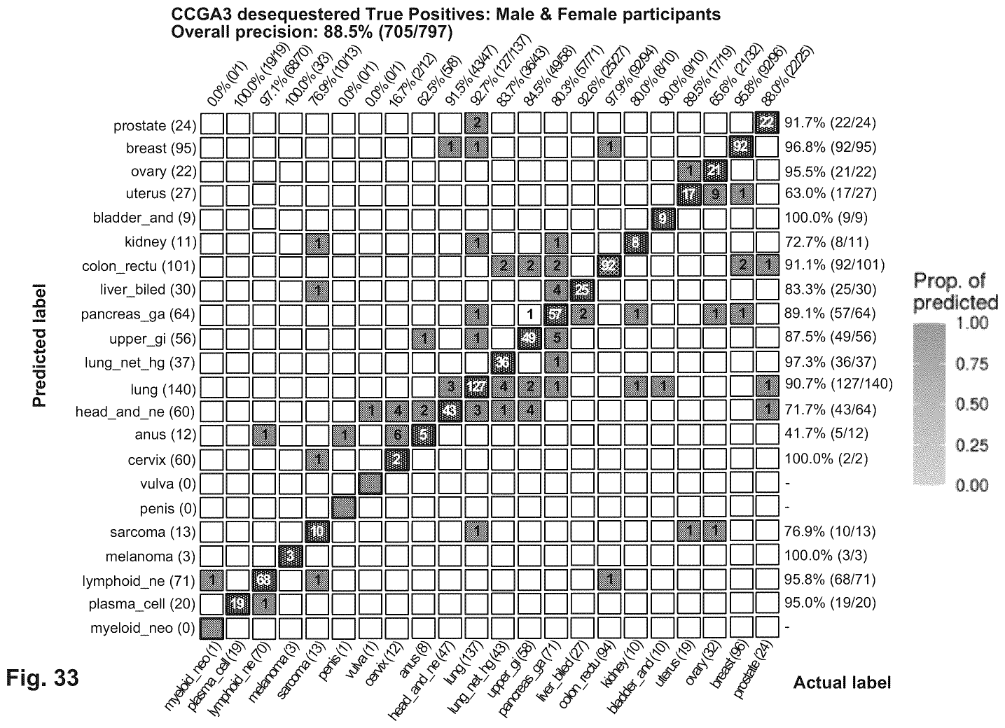

In some embodiments, the analytics system 200 trains a classifier to predict a cancer signal origin (tissue of origin) conditional on one or more characteristics (e.g., co-factors) of individuals. Example characteristics include: age, status as a smoker (e.g., individual smokes regularly, sometimes, or never), biological sex (e.g., male, or female), ethnicity, etc. By taking one or more characteristics into account, the cancer signal origin prediction of a classifier is tailored to a specific demographic inclusive of the patient rather than a general population of all individuals. As a result, the classifier may be less likely to generate false positives and may reduce crosstalk between different types of cancer signal origin, which is further described below with reference to FIGS. 33-35. Likewise, false positives and cross-talk between predictions of cancer signal origin can be distributed more effectively among results of the classifier for patients. For example, male breast cancer is approximately 100× less likely than female breast cancer, so it may be important to reduce predictions of breast CSO in males, redistributing those predictions to other CSO predictions where errors won't overshadow the incidence of true cancers. By reducing false positives that are assigned to low incidence cancers and transferring the classifications to high incidence cancers, the classifier can improve its precision of predictions, i.e., the percentage of samples labeled with a cancer signal origin that actually have that predicted cancer signal origin. A classifier with high precision increases the likelihood that a patient will obtain the proper intervention for the type of cancer they actually have and not receive unnecessary intervention for a false positive. Even a small improvement in precision or accuracy can be significant for detection and treatment of low frequency cancers (those with rare occurrences).

In an embodiment, the analytics system 200 takes one or more characteristics into account by training separate classifiers for different combinations of the characteristics. For example, the analytics system 200 trains four different classifiers based on sex (male or female) and status as a smoker (smoker or never-smoker). In this example, (1) a first classifier is trained using data from samples of individuals who are female and never-smokers, (2) a second classifier is trained using data from samples of individuals who are female and smokers, (3) a third classifier is trained using data from samples of individuals who are male and never-smokers, and (4) a fourth classifier is trained using data from samples of individuals who are male and smokers. Due to the different sets of training data used to train the four classifiers, each of the trained classifiers has different latent weights personalized to the specific characteristics. Moreover, the analytics system 200 can adjust the weights of an existing classifier using a specific set of training data associated with one or more characteristics.

In a different embodiment, the analytics system 200 takes one or more characteristics into account by applying one or more post-processes to an output of a classifier. As one example, the analytics system 200 multiplies a classifier's output probability by a factor based on a characteristic. For instance, the factor is based on an age of the subject because a certain cancer signal origin is more likely to occur in older individuals. As another example, the factor increases the probability of lung cancer signal origin if the subject is a smoker and decreases the output probability if the subject is a never smoker.

FIG. 33 illustrates predicted and actual labels of cancer signal origin for a set of male and female individuals, according to one embodiment. The y-axis indicates cancer signal origins predicted by a classifier that has not been trained to generate predictions conditional on one or more characteristics. The classifier generated the predictions for samples of 797 individuals including both male and female individuals. The x-axis indicates the actual known cancer signal origin for the 797 samples, resulting in an overall precision of 88.5%. Although the classifier has a high level of precision for certain cancers, the level of precision could be improved for other cancers.

For example, out of the 12 samples predicted to have anus cancer, only 5 of the samples actually had anus cancer, while 6 of the incorrect predictions actually had cervix cancer. Since male individuals do not have a cervix, the classifier could improve the precision of predictions if the classifier was personalized by sex. Specifically, a classifier conditional on male sex that is trained using data from samples of male individuals only would not generate predictions of cervix cancer. Since the classifier would not make the incorrect predictions labeling 6 of the cervix cancer samples as anus cancer, the classifier's precision for anus cancer would increase from 5/12 (41.7%) to 5/6 (83.3%).

As another example, FIG. 33 shows that the classifier made 140 predictions of lung cancer, where 127 of those predictions were accurate and 13 predictions were inaccurate. The likelihood of lung cancer for non-smokers is approximately a magnitude less than the likelihood for smokers. Thus, a classifier training using samples of smokers would generate predictions with greater precision than a classifier trained using samples of non-smokers. The accurate predictions of lung cancer in non-smokers (approximately 127/10=12.7) may be difficult to distinguish from the crosstalk of the 13 incorrect predictions of lung cancer that actually had a different cancer signal origin.

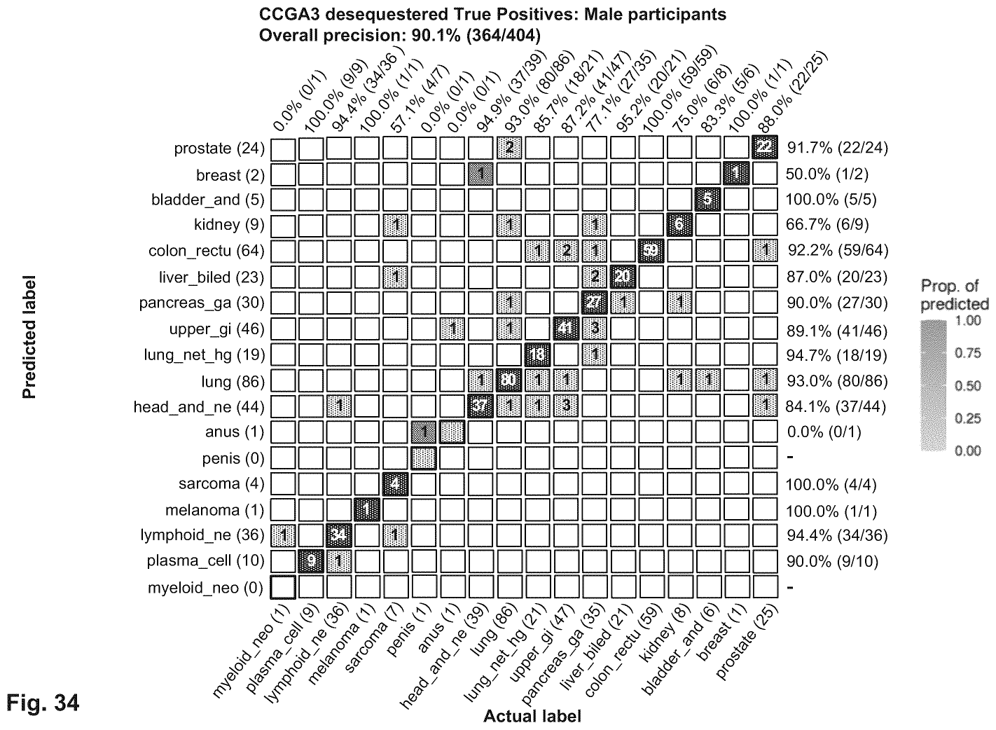

FIG. 34 illustrates predicted and actual labels of cancer signal origin for a set of male individuals, according to one embodiment. The classifier correctly predicted the one breast cancer label, but also generated a false positive breast cancer prediction that was actually head and neck cancer. Since breast cancer is rare in male individuals, a conditional classifier conditioned on the male sex would have improved performance for breast cancer predictions. A conditional classifier trained using samples from male individuals only (no samples from female individuals) results in a classifier with a more stringent threshold or requirement to return a breast cancer prediction.

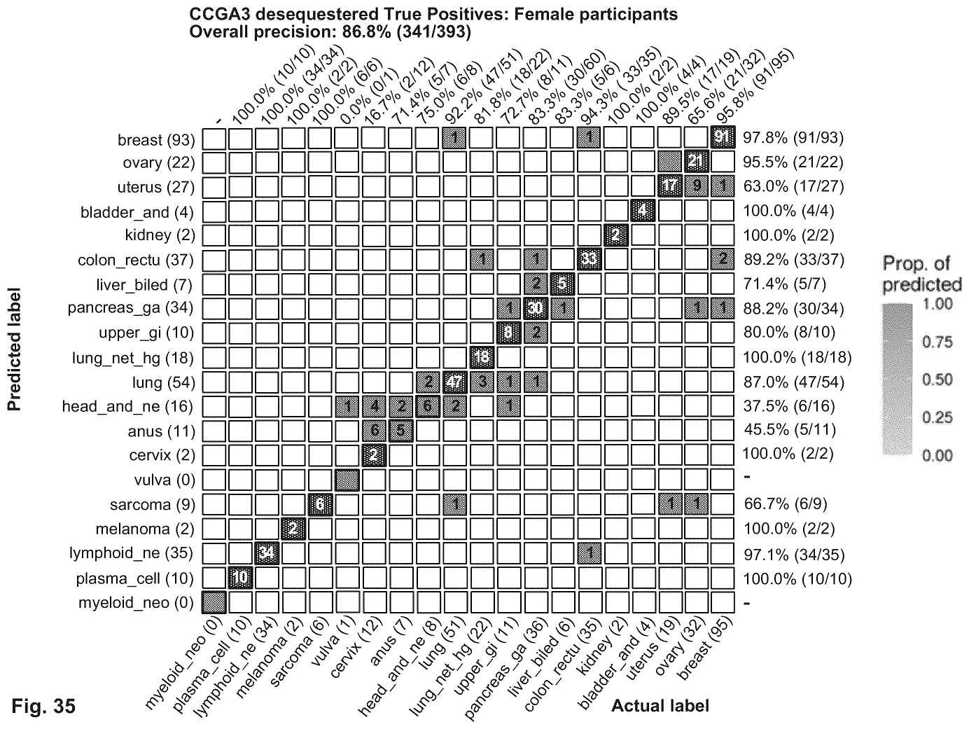

FIG. 35 illustrates predicted and actual labels of cancer signal origin for a sample of female individuals, according to one embodiment. The classifier made cervix cancer predictions with 100% precision (2/2), but the 16.7% accuracy (2/12) is low because 10 samples that actually had cervix cancer were labeled with a different cancer signal origin (head and neck or anus cancer). Since cervix cancer does not occur in male individuals, a classifier conditional on female sex would have an improved accuracy of cervix cancer predictions.

### III.H. Optimizing Features

In some embodiments, the analytics system 200 performs one or more processes to optimize feature selection when training classifiers. By optimizing the selection of features that are more informative and have lower noise, the analytics system 200 can improve the sensitivity of a trained classifier, for instance for one or more or all cancer types. An example process to optimize feature selection is using a subset of the top ranked features for the classifier. The ranks of features are inversely proportional to mutual information. Accordingly, features with a low rank have a high mutual information, making them useful for classifying different types of cancer. In some embodiments, the analytics system 200 selects a certain number of the top features. For example, the analytics system 200 selects the 256 features with the lowest (best) rank per cancer-pair group by positive type, while the remaining features outside the top 256 per cancer-pair group are not used for the classifier. The threshold of 256 features per cancer-pair group is a hyperparameter for training the classifier. In other embodiments, the threshold is a different number (e.g., 128, 256, 512, 1024) of features, which is generally referred to as “TopK.” The TopK threshold can be determined experimentally, using machine learning, or using another type of process.

To determine the TopK threshold for a conditional classifier trained for a particular demographic of individuals, the analytic system may train parallel classifiers based on featurization of the training datasets with a different number of top informative features. The analytics system may thereby modify each training samples feature vector based on the number of top informative features being evaluated. For example, the analytics system trains a first classifier evaluating the top 128 features, a second classifier evaluating the top 256 features, a third classifier evaluating the top 512 features, and a fourth classifier evaluating the top 1024 features. The analytics system may validate the accuracy of each classifier with a validation set of training samples. The classifier with the optimal accuracy is selected for the particular demographic of individuals.

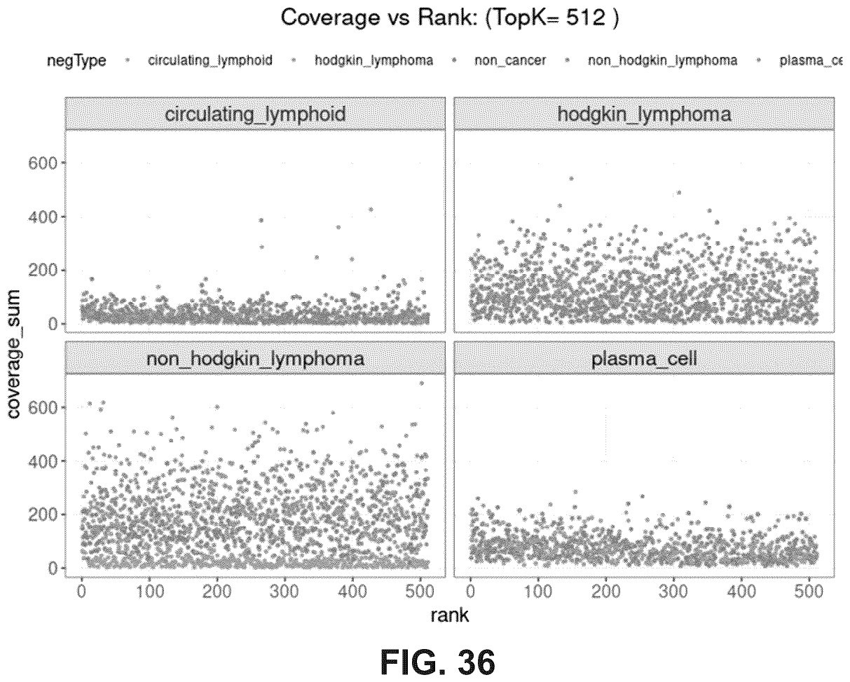

FIG. 36 illustrates experimental results of fragment coverage versus feature rank, according to one embodiment. The analytics system 200 determines the “coverage_sum” as the sum of the mean coverage of positive type fragments and the mean coverage of the negative type fragments. As shown in FIG. 36, the mean coverage per sample varies based on the cancer type (e.g., circulating lymphoid, Hodgkin lymphoma, non-Hodgkin lymphoma, and plasma cell). In some situations not shown in FIG. 36, samples with low coverage correspond to noisier features.

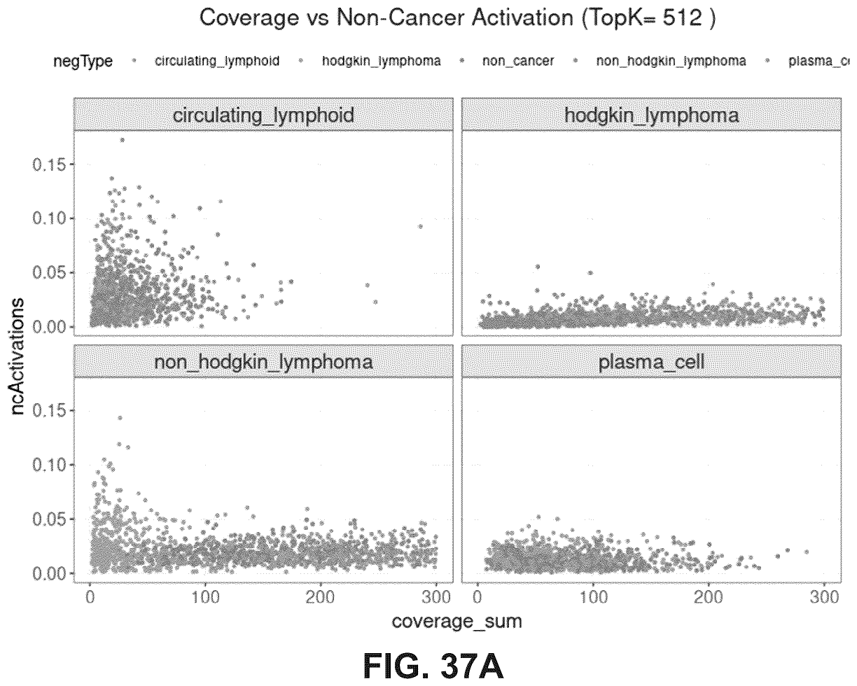

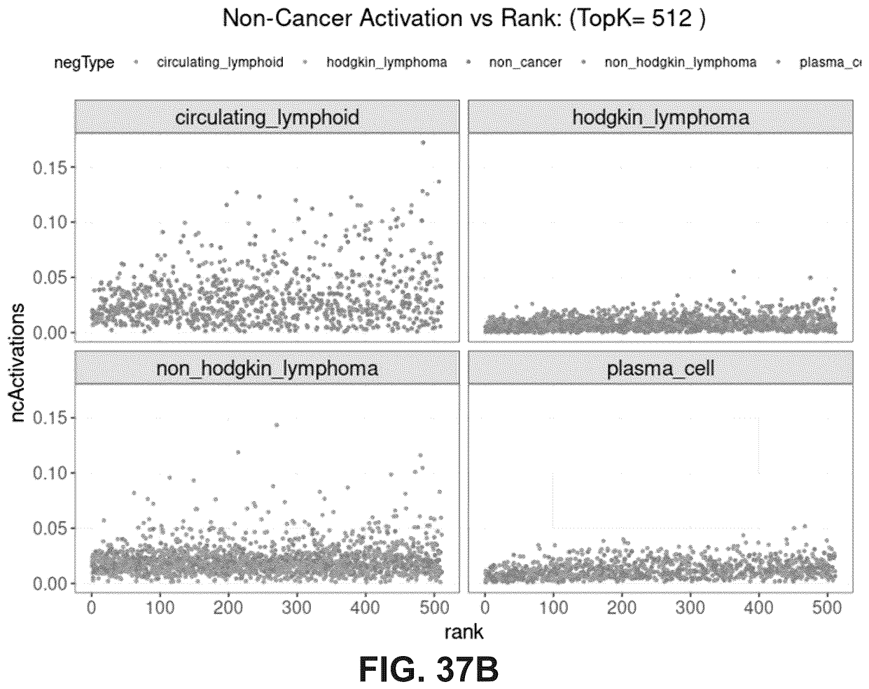

FIG. 37A illustrates experimental results of fragment coverage versus non-cancer activation fractions, according to one embodiment. FIG. 37B illustrates experimental results of non-cancer activation fraction versus feature rank, according to one embodiment. The analytics system 200 determines the non-cancer activation fractions as the portion of non-cancer samples with a certain feature turned on (e.g., ncActivations=sum(binarized(featureVals) for non-cancer samples)/len(non-cancer samples)). The analytics system 200 uses non-cancer activation fractions as a measure of noise because a greater non-cancer activation fraction value can mean that the samples have greater noise, which is less useful for a classifier to distinguish cancer types. Ideally, the non-cancer activation fraction value would be close to zero to have a low noise level. In another embodiment, analytics system 200 uses the difference between cancer and non-cancer activation fractions as a measure of noise. It is advantageous to select features that do not turn on in non-cancer samples (but turn on in cancer samples), so that the trained classifier is able to make more accurate predictions (discerning cancer from non-cancer) based on the top features. As shown in FIG. 37A, the data samples for circulating lymphoid and non-Hodgkin lymphoma have greater noise at lower coverage. In comparison, the data samples for Hodgkin lymphoma and plasma cell have lower noise in both FIGS. 37A and 37B.

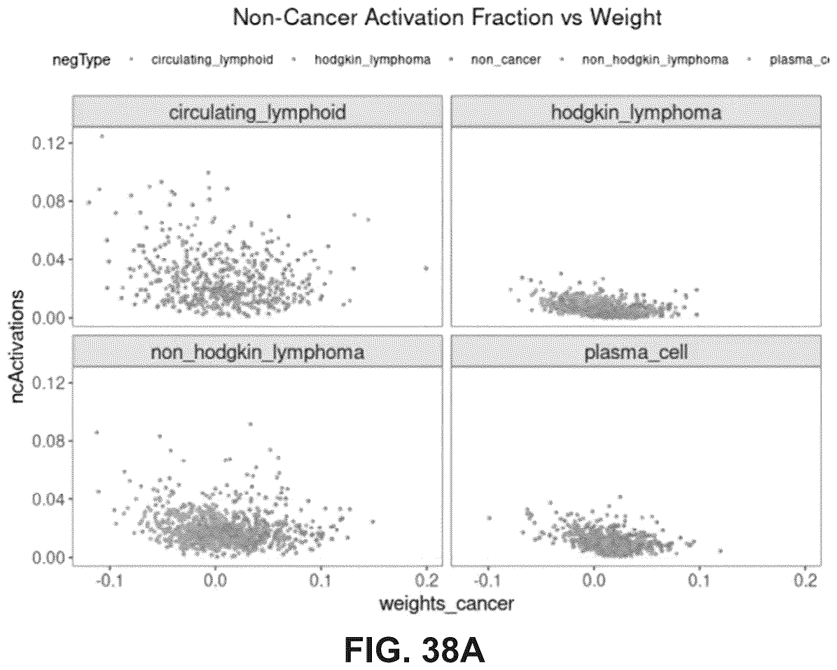

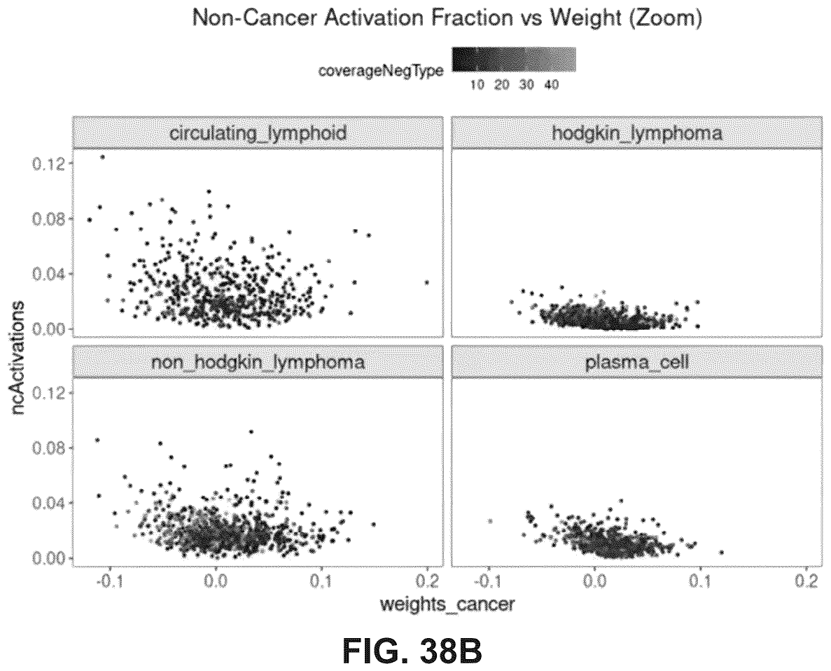

FIG. 38A illustrates experimental results of non-cancer activation fraction versus feature weight, according to one embodiment. FIG. 38B illustrates another view of the experimental results shown in FIG. 38A, according to one embodiment. The weight indicates the extent to which a certain feature contributes to a p-cancer score output by a classifier.

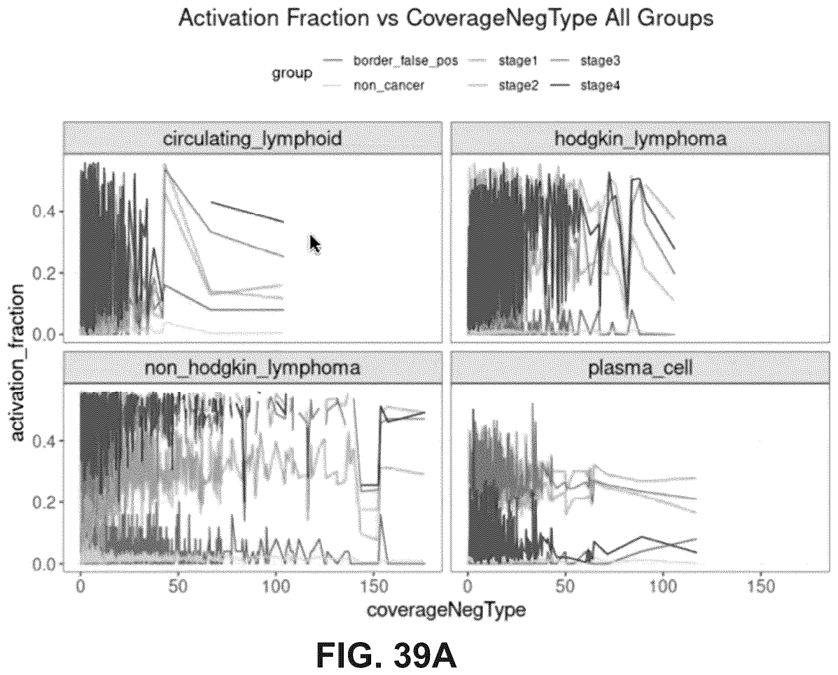

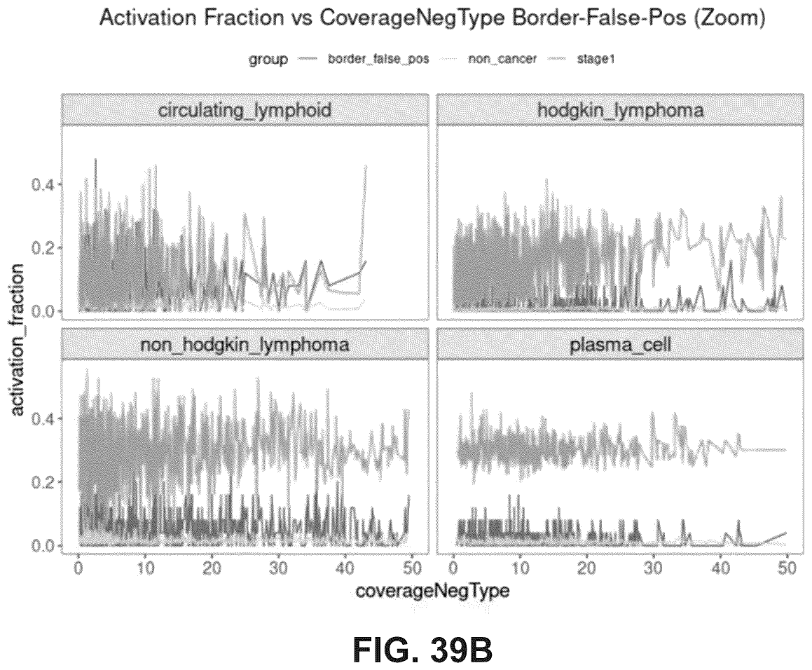

FIG. 39A illustrates experimental results of activation fraction versus coverage, according to one embodiment. FIG. 39B illustrates another view of the experimental results shown in FIG. 39A, according to one embodiment. FIGS. 39A-B show that stage 4 cancer generally has a greater activation fraction than non-cancers and the other cancer stages, which enables a trained classifier to discern stage 4 cancer. In contrast, it is more difficult to discern activation fractions between stage 1 cancer and borderline false-positive detections because their activation fractions are similar. The analytics system 200 determines the borderline false-positive detections based on p-cancer threshold such as 0.1, though the cut off threshold can vary.

As previously discussed, the analytics system 200 generates features by cancer-pair, which includes a positive type (with a certain type of cancer) and negative type (without the certain type of cancer). The analytics system 200 calculates a mutual information score for the cancer pairs and determines a threshold with the highest mutual information score. The analytics system then returns a set of features for each region of fragments, where the set includes a feature for each cancer-pair. For example, given 21 different types of cancer types (plus non-cancer), up to 21×21 features are returned per region (non-cancer is not used as a positive type). The analytics system 200 ranks the features by the mutual information score within the cancer-pair groups. Next, the analytics system 200 filters the ranked features using the TopK threshold and applies deduplication.

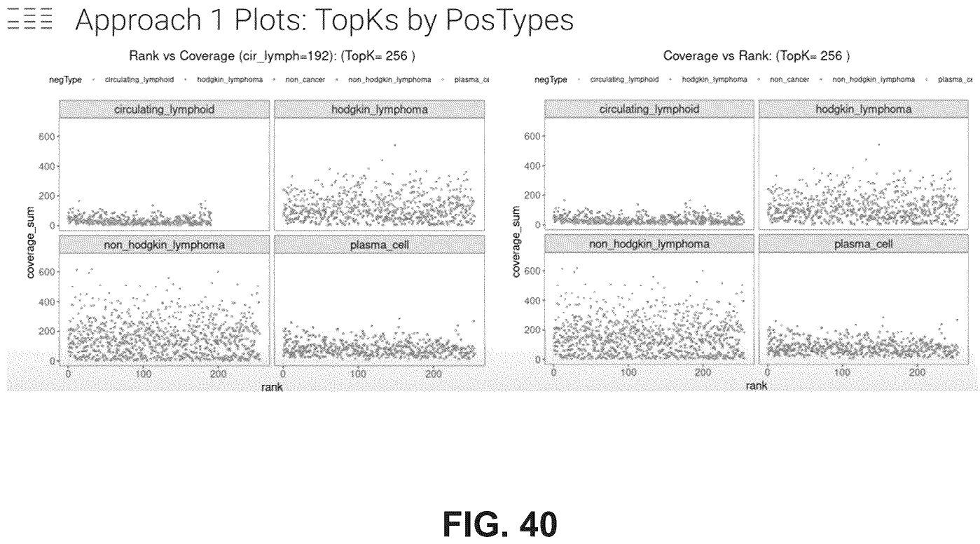

In an embodiment, the analytics system 200 adjusts the TopK threshold based on the positive cancer type. As one implementation, the analytics system 200 uses a lower TopK threshold for positive cancer types that have a higher level of noise. For example, the analytics system 200 applies a TopK threshold of 192 to the circulating lymphoid positive cancer type and a TopK threshold of 256 to the Hodgkin lymphoma, non-Hodgkin lymphoma, and plasma cell positive cancer types. Alternatively, the analytics system 200 adjusts the TopK threshold based on positive cancer type sample availability. FIG. 40 illustrates experimental results of this embodiment.

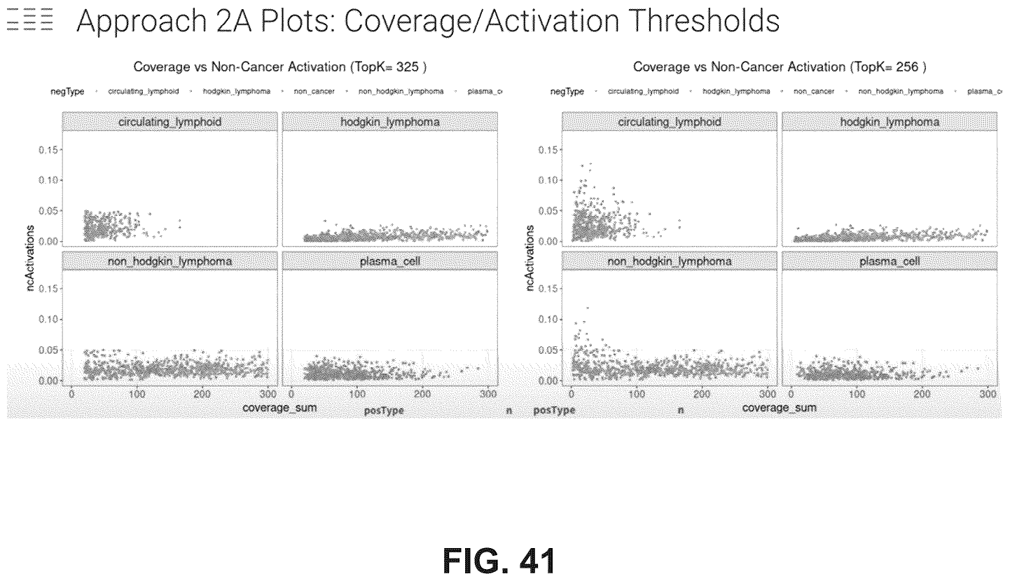

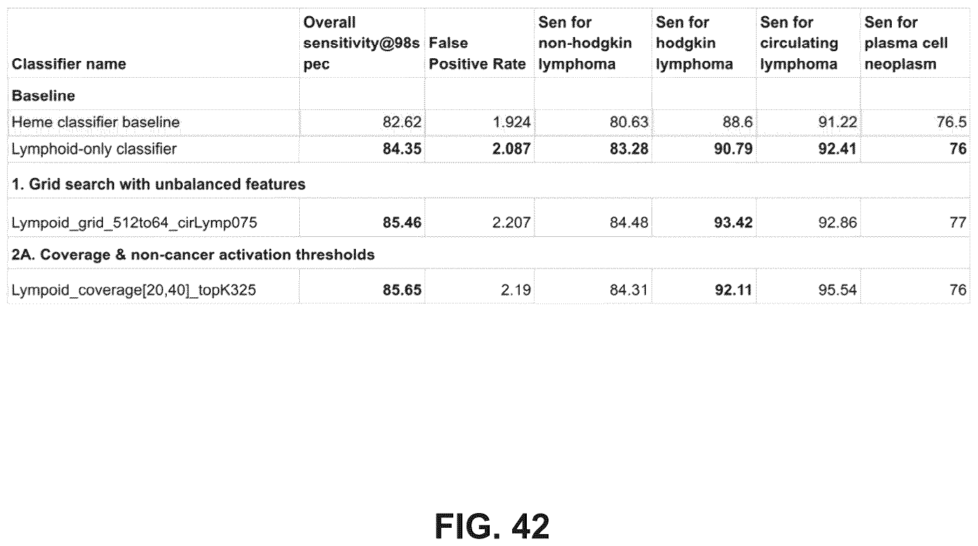

In an embodiment, the analytics system 200 prioritizes features using statistics including one or more of coverage (either coverage_sum or coverage_negative_type), activation fractions (non-cancer and/or cancer minus non-cancer), and mutual information score. As described above, features with high mutual information are useful for classifying different types of cancer. Features with low coverage are less desirable for training a classifier because they can have high levels of noise compared to features with greater coverage. Features with low non-cancer activation fractions are more desirable due to their low noise levels. Similarly, features with large differences between cancer and non-cancer activation fractions are more desirable due to their stronger signal quality. The analytics system 200 takes this into account by ranking features using mutual information score, coverage, and activation fractions (non-cancer and/or cancer minus non-cancer). In one example, for each cancer-pair group, the analytics system 200 applies thresholds for a minimum coverage level and maximum non-cancer activation fraction level. The minimum coverage level and activation fraction thresholds can vary by TopK within each positive cancer type. As another example, the analytics system 200 ranks features using a function: rank=α*mutual information score+β*coverage+γ*non-cancer activation fraction+delta*(cancer minus non-cancer activation fraction), where α, β, γ, and delta are different weights. In embodiments where features are initially ranked using mutual information score only or another criterion, the analytics system 200 can re-rank the features using additional statistics. The analytics system 200 applies a TopK threshold to filter the features after the ranking or re-ranking. FIG. 41 illustrates experimental results of this embodiment. FIG. 42 illustrates experimental results using the different thresholding methods described above, according to various embodiments.

In an embodiment, the analytics system 200 generates a statistical model (e.g., Z-score) to identify outlier features based on mutual information. For example, for each cancer-pair group, the analytics system 200 estimates the distribution of mutual information scores and determines a cutoff threshold based on that distribution. The statistical model can also account for regional fragment coverage or activation fraction statistics.

In an embodiment, the analytics system 200 uses the same “optimized” features described above for both the binary and the TOO classification stages. In another embodiment, different sets of “optimized” features are used for the binary and the TOO classification stages.

### IV. MULTILAYER PERCEPTRON MODEL

In some embodiments, a multilayer perceptron model (“MLP”) can be used as an alternative to logistic regression for classification. As with the logistic regression based classifier, the MLP classifier can be a single multi-class classifier for both detecting cancer and determining a cancer tissue of origin (TOO) or cancer type. For example, the multi-class classifier can be trained to distinguish two or more, three or more, five or more, ten or more, fifteen or more, or twenty or more different types of cancer. In one embodiment, the multi-class cancer MLP model can also include a class label for non-cancer, and cancer detection can be determined (e.g., as 1-non-cancer). In another embodiment, the multilayer perceptron model can be a two-stage classifier having a first stage for binary classification (e.g., cancer or non-cancer), and a second stage multilayer perceptron model for multi-class classification (e.g., TOO), e.g., with one or more hidden layer.

In one embodiment, the multilayer perceptron comprises a two-stage classifier: a first stage multilayer perceptron (MLP) binary classifier with no hidden layer; and a second stage multilayer perceptron (MLP) multi-class classifier with a single hidden layer. In one embodiment, sample determined to have cancer using the first stage classifier will subsequently analyzed by the second stage classifier.

In the first stage of training, a binary (two-class) multilayer perceptron model with no hidden layers for detecting the presence of cancer can be trained to discriminate cancer samples (regardless of TOO) from non-cancer. For each sample, the binary classifier outputs a prediction score indicating the likelihood of a presence or absence of cancer.

In the second stage of training, a parallel multi-class multilayer perceptron model for determining cancer type or cancer tissue of origin can be trained. In one embodiment, only cancer samples that received a score above a cutoff threshold (e.g., the 95th percentile of the non-cancer samples in the first stage classifier) can be included in the training of this multi-class MLP classifier. For each cancer sample used in training and testing, the multi-class MLP classifier outputs prediction values for the cancer types being classified, where each prediction value is a likelihood that the given sample has a certain cancer type. For example, the cancer classifier can return a cancer prediction for a test sample including a prediction score for breast cancer, a prediction score for lung cancer, and/or a prediction score for no cancer.

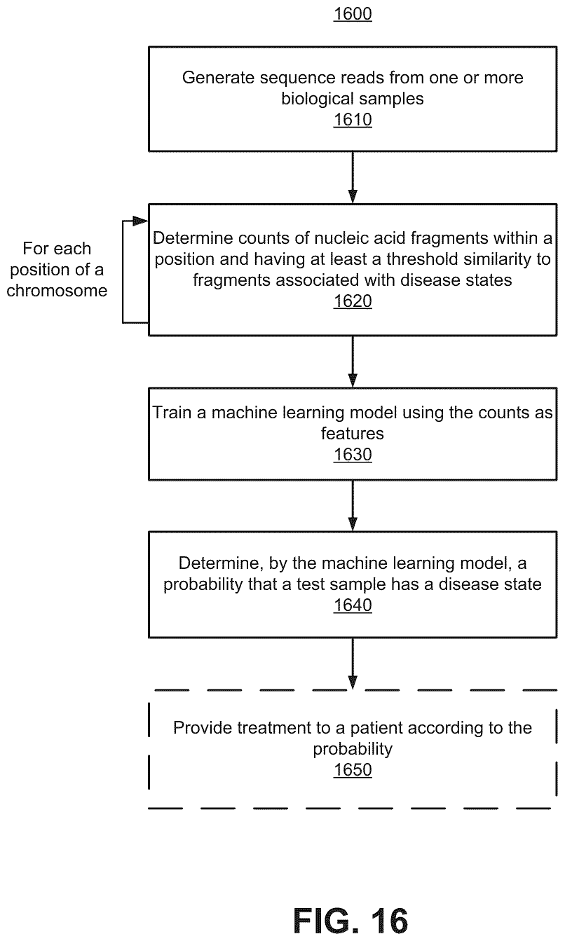

FIG. 16 is a flowchart of a method 1600 for determining a probability that a sample has a disease state according to various embodiments. In some embodiments, the analytics system 200 performs the method 1600 to process sequence reads of fragments from nucleic acid samples. The method 1600 includes, but is not limited to, the following steps, which are described with respect to the components of the analytics system 200.

In step 1610, the analytics system 200 generates sequence reads from one or more biological samples. In some embodiments, the analytics system 200 filters the sequence reads according to p-value scores of the sequence reads. The p-value score of a sequence read indicates a probability of observing methylation in a nucleic acid fragment of the one or more biological samples corresponding to the sequence read.

In step 1620, the analytics system 200 uses the sequence reads to determine, for each position of a set of positions of a chromosome, counts of nucleic acid fragments of the one or more biological samples within the position and having at least a threshold similarity to fragments associated with disease states, e.g., cancer-like fragments. The disease state may be associated with at least one type of cancer, a stage of cancer, or another type of disease or condition.

Each of the positions may represent a number of continuous base pairs of the chromosome. The number of base pairs may vary between different positions. The analytics system 200 may generate the sequence reads for multiple regions of a genome. There can be up to tens of thousands or more regions. Each region may include hundreds, thousands, or more base pairs. The method 1600 may be performed for whole-genome bisulfite sequencing (WGBS) or for a targeted panel assay.

In step 1630, the analytics system 200 trains a machine learning model using the counts of the positions as features. In some embodiments, the analytics system 200 binarizes the features to indicate a presence or absence (e.g., Boolean value) of one of the disease states in each of the positions. A count of at least one nucleic acid fragment in a position indicates presence of one of the disease states in the position. A count of zero nucleic acid fragments in a position indicates absence of one of the disease states in the position. In some embodiments, the machine learning model can be a logistic regression model. In some embodiments, the machine learning model can be a multilayer perceptron model (neural network). As one of skill in the art would readily appreciate other machine learning models can be used, including, for example, generalized linear model (GLM), multilayer perceptron, support vector machine, random forest, or neural network classifier.

In step 1640, the trained machine learning model determines a probability that a test sample has a disease state. The test sample can be obtained from a patient and can include blood and/or tissue. In an optional step 1650, treatment is provided to the patient according to the probability. For example, the patient can be provided treatment (e.g., medication or interventional procedure) responsive to determining that the probability is greater than a threshold value. In another embodiment, in optional step 1650, a test report can be generated to provide the patient with their test results, including a probability that the test sample has a disease.

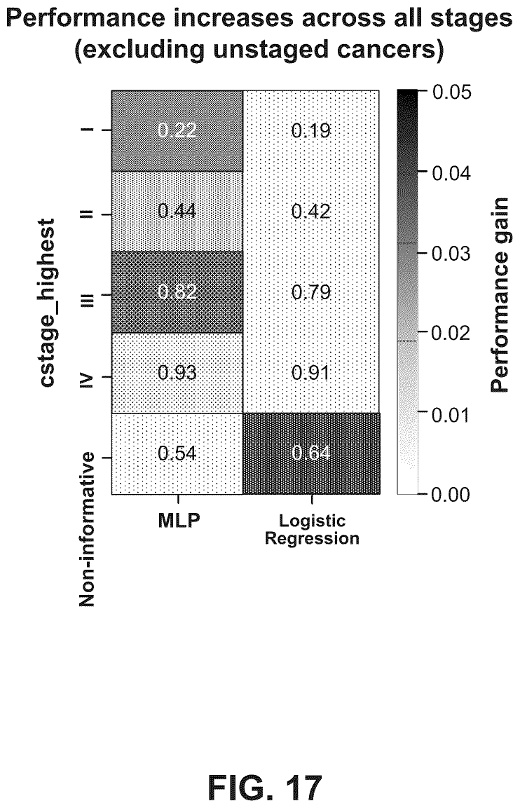

The experimental results shown in FIGS. 17-20 were obtained by training models using samples from the CCGA study, which is further described below.

FIG. 17 illustrates performance gain in sensitivity of a multilayer perceptron model according to an embodiment. In comparison to a logistic regression model, the multilayer perceptron model (MLP) demonstrates performance gains in sensitivity of disease detection across cancer stages I, II, III, and IV.

FIG. 18 illustrates experimental results of a multilayer perceptron model in determining tissue of origin according to an embodiment. In comparison to a logistic regression model (LR: 1803 and 1804), the multilayer perceptron model (MLP: 1801 and 1802) has improved accuracy in determining tissue of origin. The improved accuracy is realized when processing sequence reads associated with all cancer types of a training set, as well as when processing sequence reads of a training set including more than 10 example sequence reads for each cancer type in the training set.

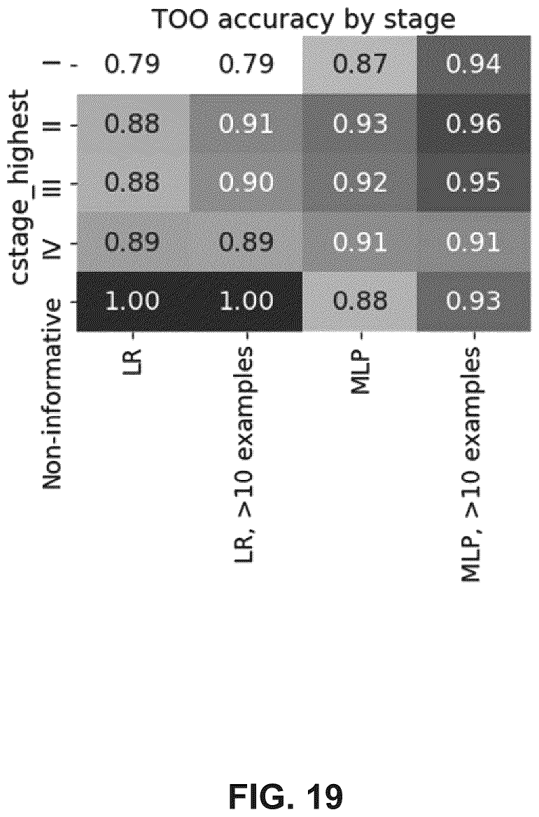

FIG. 19 illustrates experimental results of a multilayer perceptron model in determining tissue of origin by cancer stage according to an embodiment. In comparison to a logistic regression (LR) model, the multilayer perceptron model (MLP) demonstrates performance gains in accuracy of tissue of origin (TOO) detection across cancer stages I, II, III, and IV. Among the cancer stages, the performance gain for the MLP model is greatest for stage I.

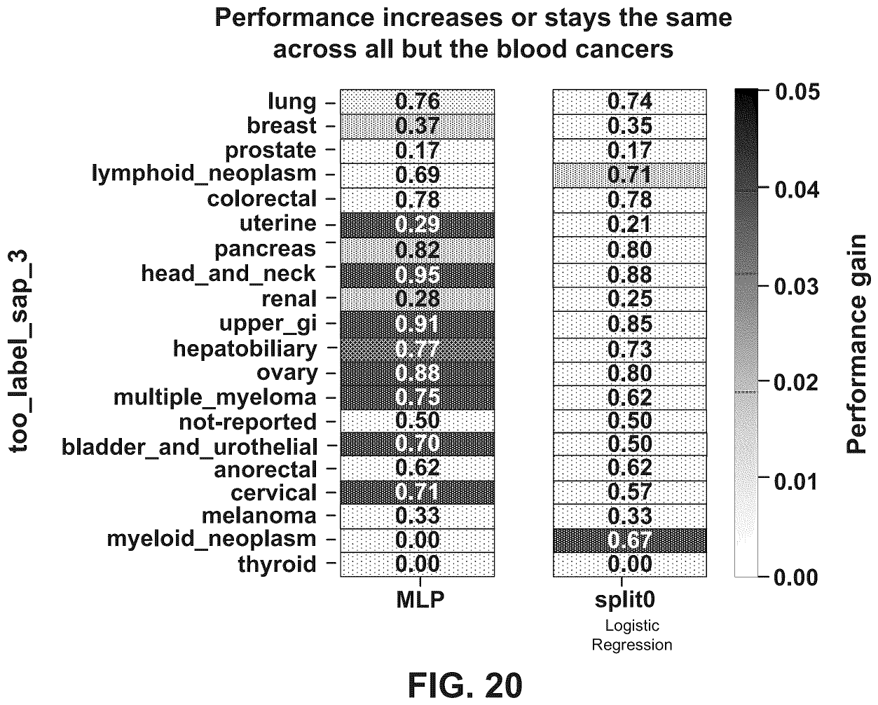

FIG. 20 illustrates experimental results of a multilayer perceptron model across types of cancers according to an embodiment. For most of the types of cancers shown in FIG. 20, the multilayer perceptron model (MLP) achieves greater accuracy in tissue of origin (TOO) detection in comparison to a logistic regression model.

In some embodiments, the analytics system uses a two-stage model to determine a tissue of origin (TOO) of cancer or another type of disease state. The analytics system generates sequence reads from nucleic acid fragments of biological samples. The analytics system determines a first set of training data by processing the sequence reads, for example, using any of the processes described in Section II. A. Assay Protocol. The analytics system can use methylation information to determine the first set of training data. For instance, the analytics system determines sequence reads that are hypomethylated by determining that a threshold number or percentage of CpG sites corresponding to the sequence reads are unmethylated. In addition, the analytics system determines sequence reads that are hypermethylated by determining that a threshold number or percentage of CpG sites corresponding to the sequence reads are methylated. The analytics system can also determine that sequence reads are informative. In some embodiments, the analytics system filters the sequence reads by removing sequence reads having a p-value less than a threshold p-value.

The analytics system trains a binary classifier using the first set of training data. The binary classifier is trained to predict, for an input sequence read from a first test biological sample, a binary output, that is, the presence or absence of at least one disease state in the first test biological sample.

Using predictions of the binary classifier, the analytics system can determine that a subset of the biological samples has a presence of one or more disease states. The binary classifier can be used to train a tissue of origin classifier. In particular, the analytics system determines a second set of training data using the sequence reads corresponding to the nucleic acid fragments of the subset of biological samples. The analytics system trains the tissue of origin classifier using the second set of training data. The tissue of origin classifier is trained to predict, for an input sequence read from a second test biological sample, a tissue of origin associated with a disease state present in the second test biological sample. The first and second test biological samples can be the same sample or different samples.

In some embodiments, the analytics system uses the tissue of origin classifier to determine a score indicating a probability that the tissue of origin associated with the disease state is present in the second test biological sample. The analytics system can calibrate the score, e.g., to tune the output of an over-confident model. For instance, the analytics system performs a k-nearest neighbor (KNN) operation in association with the score using a feature space output by the tissue of origin classifier. In an embodiment, the feature space includes the top two prediction labels from the tissue of origin classifier (e.g., lung cancer and prostate cancer) as well as an indication whether the correct classification was a disease state different than the top two predictions. The analytics system can also calibrate the score by normalizing the probability using an output of the binary classifier indicating a different probability of a presence of the at least one disease state present in the second test biological sample.

In some embodiments, the tissue of origin classifier is a multilayer perceptron including at least one hidden layer. The tissue of origin classifier can also include a 100-unit hidden layer or a 200-unit hidden layer, among other sizes of hidden layers. The multilayer perceptron can be fully connected and use a rectified linear unit activation function. In some embodiments, the binary classifier is a multilayer perceptron that does not include a hidden layer. In a different embodiment, the binary classifier is a multilayer perceptron including at least one hidden layer. In other embodiments, these classifiers can be a logistic regression model, multinomial logistic regression model, or other types of machine learning models.

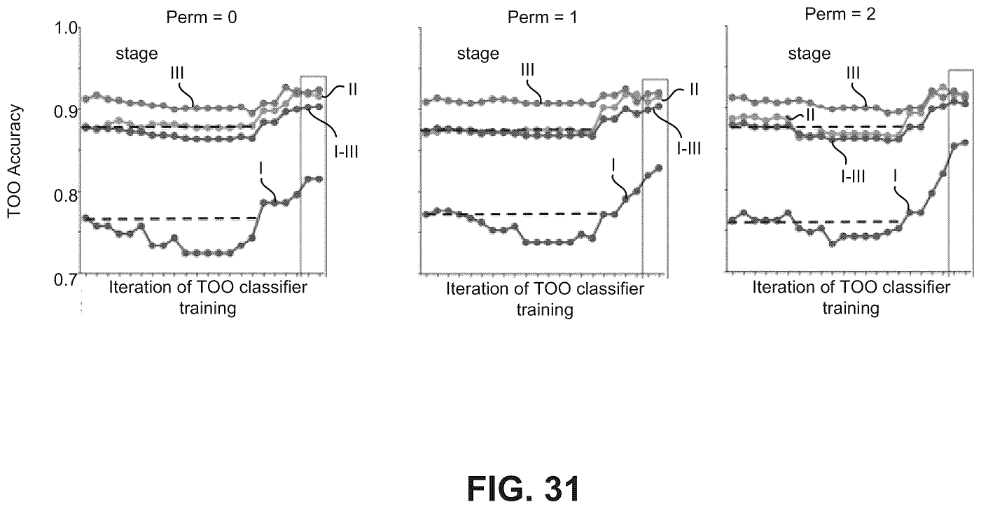

Moreover, the analytics system can train the tissue of origin classifier and the binary classifier using one or more machine learning techniques known to one skilled in the art including, for example, no early stopping (instead selecting a given number of training epochs), stochastic gradient descent, weight decay, dropout regularization, Adam optimization, He initialization, and learning rate scheduling, rectified linear unit activation function, leaky rectified linear unit activation function, sigmoid activation function, and boosting, among others. As shown in FIG. 31, the tissue of origin accuracy of the tissue of origin classifier improves over training iterations. The iterations may each include a different combination of the machine learning techniques. Additionally, the increase of tissue of origin accuracy is present across different cancer stages: I, II, and III.

In some embodiments, the analytics system performs cross validation on one or both of the tissue of origin classifier and the binary classifier. The analytics system can retrain a classifier using hyperparameters selected based on the output of cross-validation. The analytics system can select the hyperparameters by aggregating results from all folds in the cross-validation. In an embodiment, the analytics system selects hyperparameters to train the tissue of origin classifier by optimizing for tissue of origin accuracy instead of log likelihood because the classifier can be more confident about samples with stronger signals.

In some embodiments, the analytics system determines, by the tissue of origin classifier, a probability that the tissue of origin associated with the disease state is present in the second test biological sample. The analytics system predicts that the tissue of origin associated with the disease state is present in the second test biological sample responsive to determining that the probability is greater than a tissue of origin threshold. The analytics system can determine different tissue of origin thresholds associated with different tissues of origin. Additionally, the analytics system can determine a tissue of origin threshold associated with a given disease state by iterating through a range of different probabilities of candidate tissue of origin thresholds. For each iteration, the analytics system determines a sensitivity rate at a given specificity rate of the tissue of origin classifier. The analytics system can optimize a tradeoff between sensitivity rate and specificity rate of the tissue of origin classifier for the given disease state. The analytics system can determine the sensitivity rate using scores output by the binary classifier or the tissue of origin classifier. Furthermore, the analytics system can stratify samples using scores from the tissue of origin classifier.

In some embodiments, the analytics system trains the binary classifier and tissue of origin classifier using binarized features each having a value of 0 or 1. Values greater than 1 are replaced with 1 in binarization.

### V. TUNING OF BINARY CLASSIFICATION THRESHOLD

The analytics system may tune the trained cancer classifier to prune samples used in training the cancer classifier. In particular, the analytics system may seek to remove non-cancer samples with high tissue signal that dilute the cancer classifier's sensitivity in cancer prediction. High tissue signal refers to a sample having a significant fraction of cfDNA from a tissue of origin (TOO), e.g., determined by a tissue of origin classifier, a multiclass cancer classifier or other means, compared to a healthy distribution. Non-cancer samples with high tissue signal are outliers in the non-cancer distribution, and they may be pre-stage cancer, early stage cancer, or undiagnosed cancer. The analytics system can identify non-cancer samples with high tissue signal in at least one cancer type. In some embodiments, certain cancer types are further separated into cancer sub-types. For example, the hematological cancer type can further be separated into a combination of, for instance, circulating lymphoid sub-type, non-Hodgkin's-Lymphoma (NHL) indolent sub-type, NHL aggressive sub-type, Hodgkin's-Lymphoma (HL) sub-type, myeloid sub-type, and plasma cell sub-type.

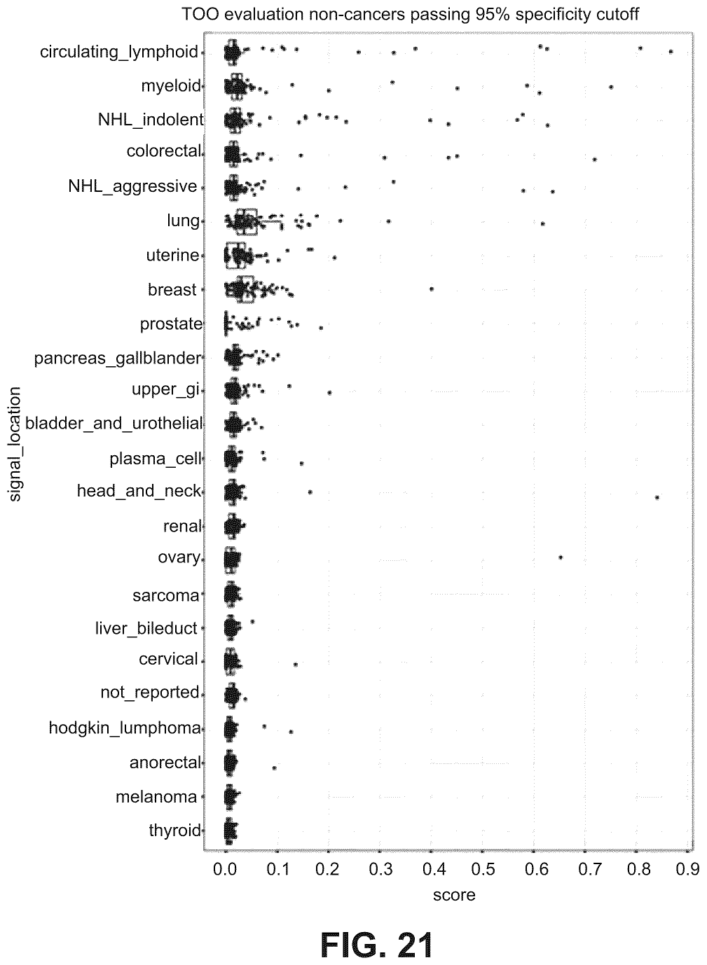

Referring to FIG. 21, FIG. 21 illustrates a graph of cancer type likelihood for non-cancer samples above 95% specificity. A cancer score was calculated for each non-cancer sample from a plurality of non-cancer samples, i.e., samples from healthy individuals not currently diagnosed with cancer. The cancer score can be determined by the binary classifier as a likelihood that a sample has cancer given the sample's methylation sequencing data. In other embodiments, the cancer score can be calculated according to other methods that input at least sequencing data (e.g., methylation, single nucleotide polymorphism (SNP), DNA, RNA, etc.) and output a sample's likelihood of having cancer based on the input sequencing data. One example of a classifier is a mixture model classifier. A distribution of the non-cancer samples can be generated according to the cancer scores of the non-cancer samples. A binary threshold cutoff can be set to ensure some level of binary classification specificity, e.g., a true negative rate. Typically, a high specificity cutoff is used in classifying cancer, e.g., between 90% and 99.9%, or 99.5% specificity or higher. However, many non-cancer samples, used in training the cancer classifier and just below the specificity cutoff, can have high tissue signal thereby positively biasing the binary threshold cutoff.

To demonstrate, non-cancer samples above the 95% specificity were selected and then input into a multiclass cancer classifier to determine a probability for each cancer type—or tissue of origin (TOO). The cancer types or TOO labels used in this embodiment of the multiclass cancer classifier include circulating lymphoid, myeloid, NHL indolent, colorectal, NHL aggressive, lung, uterine, breast, prostate, pancreas and gallbladder, upper gastrointestinal, bladder and urothelial, plasma cell, head and neck, renal, ovary, sarcoma, liver and bile duct, cervical, other tissues, HL, anorectal, melanoma, thyroid. The graph in FIG. 21 shows many non-cancer samples having high tissue signal from at least one tissue type. Each dot in a row for a tissue type corresponds to a tissue of origin likelihood for a non-cancer sample above the 95% specificity threshold. Notably, many tissue types have multiple non-cancer sample outliers having significant tissue contribution, not typical for non-cancer samples. This can arise when such non-cancer samples have cfDNA signals being driven by cancer-like methylation, clonal fraction, and/or rate of growth/turnover. It can be inferred that numerous non-cancer samples used in training the cancer classifier may be pre-stage cancer, early stage cancer, or undiagnosed cancer. Nonetheless, these non-cancer samples with significant tissue contribution shift the binary classification cutoff threshold up thereby decreasing sensitivity of the cancer classification, especially with samples with significant tissue signal just below the previously set binary classification cutoff threshold. In practice, such signals (e.g., corresponding to circulating_lymphoid, myeloid, and NHL_indolent) can be a major attractor of false positive determinations. Of note, circulating lymphoid, myeloid, NHL indolent, colorectal, NHL aggressive, lung, uterine, breast, prostate, pancreas and gallbladder, upper gastrointestinal, plasma cell, head and neck, cervical, HL had at least one non-cancer sample with a probability of tissue origin above 0.1. Particularly, circulating lymphoid, myeloid, NHL indolent, and NHL aggressive (all hematological sub-types) had two or more non-cancer samples with a probability of tissue origin above 0.5.

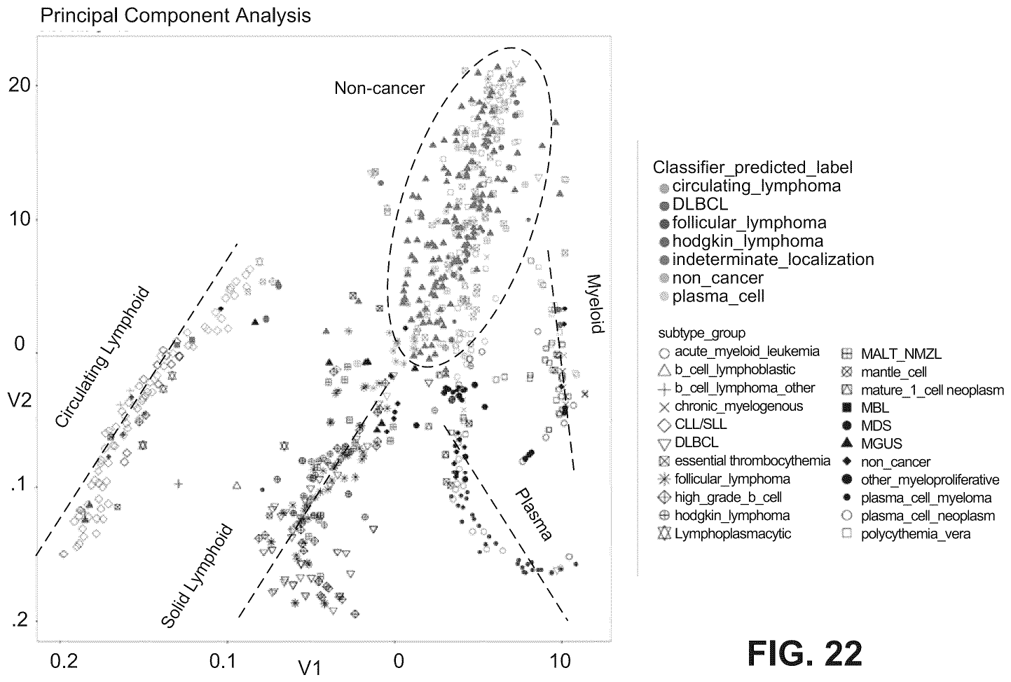

Referring to FIG. 22, FIG. 22 illustrates a graph of hematological sub-types separated according to methylation sequencing data. The graph of FIG. 22 demonstrates an ability to model hematological sub-types. This can prove beneficial in providing more granularity to the multiclass cancer classification (e.g., classifying additionally with the hematological sub-type labels) or as a manner of tuning the cancer classification through pruning non-cancer samples with high hematological sub-type signal prior to training the cancer classifier. As described above, methylation signal can cover a plurality of CpG sites, thereby creating a high-dimensional vector space. With the hematological sub-type samples and non-cancer samples, the analytics system can perform a principal component analysis. The principal component analysis identifies orthogonal principal components (or embeddings) of the vector space in order of variance in methylation signal amongst the samples. The first principal component, shown as V1 on the horizontal axis on the graph, has the highest variance with the second principal component, shown as V2 on the vertical axis on the graph, with the second highest variance. Annotated on the graph 900 are clusters of the samples for each hematological sub-type and non-cancer. The hematological sub-types shown include circulating lymphoid, solid lymphoid, plasma cell, and myeloid. The solid lymphoid sub-type can be further divided into HL, NHL indolent, and NHL aggressive. The graph shows potential for classifying according to the hematological sub-types—either for addition of the hematological sub-types in the multiclass cancer classification or for modeling each of the hematological sub-types for tuning of the cancer classifiers.

### V.a. Removal of High Signal Non-Cancer Samples

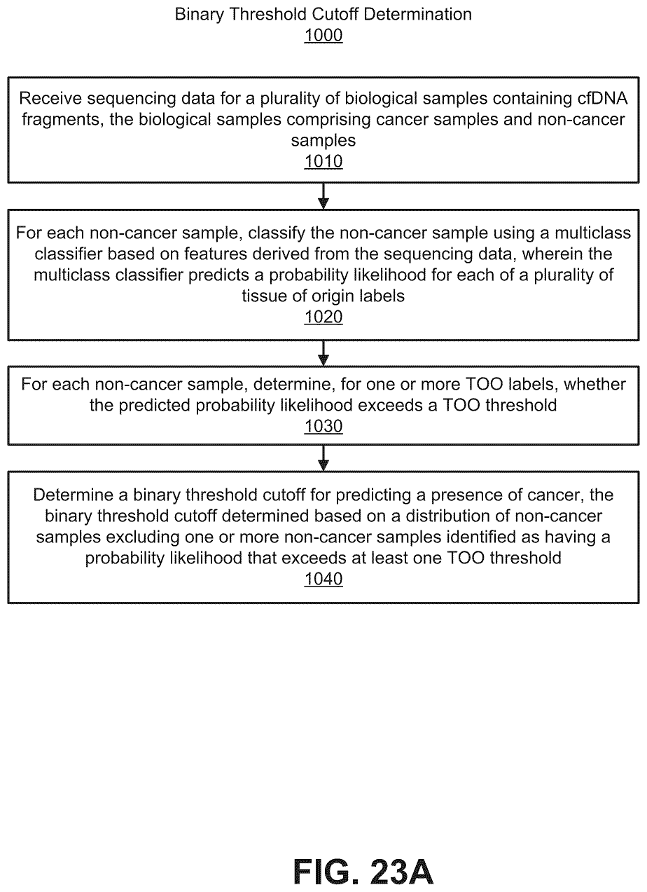

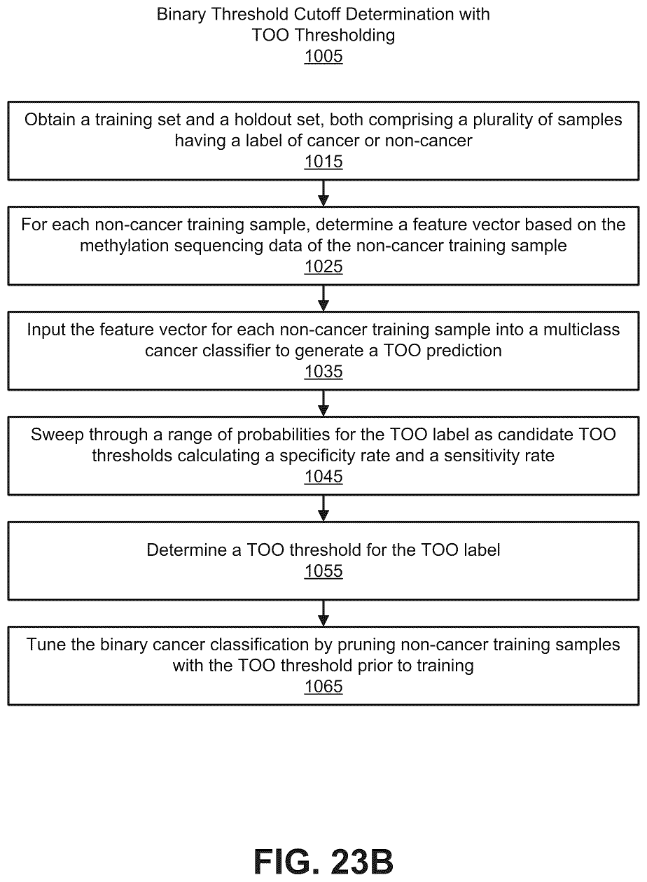

FIG. 23A illustrates a flowchart describing a process 1000 of determining a binary threshold cutoff for binary cancer classification, in accordance with one or more embodiments. A binary classification for predicting between cancer and non-cancer evaluates a sample's cancer score against a determined binary threshold cutoff, wherein a sample with a cancer score below the binary threshold cutoff is determined to be non-cancer and with a cancer score at or above the binary threshold cutoff is determined to be cancer. A trained multiclass cancer classifier evaluates a sample's methylation signal (and/or other sequencing data) to determine probabilities for a number of TOO labels classified by the multiclass cancer classifier. A TOO label used in a multiclass cancer classifier can be a cancer tissue type or a cancer tissue sub-type (e.g., the hematological sub-types described above). The process 1000 can be performed or accomplished by the analytics system.

The analytics system receives 1010 sequencing data for a plurality of biological samples containing cfDNA fragments, the biological samples comprising cancer samples and non-cancer samples. The sequencing data can be methylation sequencing data, SNP sequencing data, another DNA sequencing data, RNA sequencing data, etc.

For each non-cancer sample, the analytics system classifies 1020 the non-cancer sample using a multiclass cancer classifier based on features derived from the sequencing, wherein the multiclass cancer classifier predicts a probability for each of a plurality of TOO labels. The analytics system can generate a feature vector for the non-cancer sample, assigning an anomaly score for each CpG site in consideration based on at least one informative cfDNA fragment overlapping that CpG site.

For each non-cancer sample, the analytics system determines 1030, for one or more TOO labels, whether the predicted probability likelihood exceeds a TOO threshold. The TOO threshold determination is further described below in FIG. 23B.

The analytics system determines 1040 a binary threshold cutoff for predicting a presence of cancer, the binary threshold cutoff determined based on a distribution of non-cancer samples excluding one or more non-cancer samples identified as having a probability likelihood that exceeds at least one TOO threshold. Non-cancer samples that have at least one probability likelihood for a TOO label that exceeds the TOO threshold corresponding to that TOO label are excluded. The analytics system then calculates a distribution of the non-cancer samples according to a cancer score for each non-cancer sample and then from the distribution determines the binary threshold cutoff at a desired specificity level (e.g., 99.4-99.9% specificity). It is noted that each cancer score can be determined according to the sequencing data, e.g., the cancer score can be output by a binary cancer classifier predicting a likelihood of cancer based on methylation sequencing data, as described herein. In other embodiments, the cancer score can be calculated according to other methods that input at least sequencing data (e.g., methylation, single nucleotide polymorphism (SNP), DNA, RNA, etc.) and output a sample's likelihood of having cancer based on the input sequencing data.

FIG. 23B illustrates a flowchart describing a process 1005 of thresholding a TOO label for determining a binary threshold cutoff for binary cancer classification, in accordance with one or more embodiments. This process 1005 can be an embodiment of the process 1000. A binary classification for predicting between cancer and non-cancer evaluates a sample's cancer score against a determined binary threshold cutoff, wherein a sample with a cancer score below the binary threshold cutoff is determined to be non-cancer and with a cancer score at or above the binary threshold cutoff is determined to be cancer. A trained multiclass cancer classifier evaluates a sample's methylation signal (and/or other sequencing data) to determine probabilities for a number of TOO labels classified by the multiclass cancer classifier. A TOO label can be a cancer tissue type or more particularly a cancer tissue sub-type (e.g., the hematological sub-types described above). The process 1005 can be performed or accomplished by the analytics system.

The analytics system obtains 1015 a training set comprising a plurality of samples having a label of cancer or non-cancer and a holdout set comprising a plurality of samples having a label of cancer or non-cancer, i.e., either a cancer sample or a non-cancer sample, respectively. Each sample in the training set comprises methylation sequencing data, e.g., generated according to the process 300 of FIG. 3. In other embodiments, each training sample has other sequencing data used in tandem or in substitution of the methylation sequencing data. Moreover, each sample from the training set and the holdout set has a cancer score. As noted above, the cancer score can be determined by the binary classifier as a likelihood that a sample has cancer given the sample's methylation sequencing data. In other embodiments, the cancer score is calculated according to other methods that input at least sequencing data (e.g., methylation, single nucleotide polymorphism (SNP), DNA, RNA, etc.) and output a sample's likelihood of having cancer according to the input sequencing data, exampled by a mixture model described herein.

The analytics system, for each non-cancer training sample, determines 1025 a feature vector based on the methylation sequencing data. The analytics system can determine the feature vector for each non-cancer training sample, e.g., by determining an anomaly score for each CpG site in a set of CpG sites considered. In some embodiments, the analytics system defines the anomaly score for the feature vector with a binary score based on whether there is an informative fragment in the set of informative fragments that encompasses the CpG site. Once all anomaly scores are determined for a sample, the analytics system determines the feature vector as a vector of the anomaly scores associated with each CpG site considered. The analytics system can additionally normalize the anomaly scores of the feature vector based on a coverage of the sample.

The analytics system inputs 1035 the feature vector for each non-cancer training sample into a multiclass cancer classifier to generate a TOO prediction. The multiclass cancer classifier is trained on a plurality of TOO labels, including cancer types, cancer sub-types, non-cancer, or any combination thereof. The multiclass cancer classifier can be trained as described herein. The trained multiclass cancer classifier determines, as the cancer prediction, a plurality of probabilities for the TOO labels, wherein a probability for a TOO label indicates likelihood of having a cancer corresponding to the TOO label.

In some examples, the analytics system sweeps 1045 or iterates through a range of probabilities for the TOO label as candidate TOO thresholds calculating a specificity rate and a sensitivity rate over the range of probabilities for the TOO label. The analytics system can sweep through the range of probabilities incrementally, e.g., by 0.01, 0.02, 0.03, 0.04, 0.05, etc. As the analytics system sweeps through the range of probabilities, the analytics system filters non-cancer training samples having a probability of the TOO label at or above the candidate TOO threshold, according to the output of the multiclass cancer classifier. As a numerical example, the analytics system considers a candidate TOO threshold of 0.35. Non-cancer training samples with a probability of the TOO label at or above 0.35 are filtered out of the training set. The analytic system determines an adjusted binary threshold cutoff based on the filtered training set. The analytics system calculates a specificity rate of prediction with the adjusted binary threshold cutoff against the holdout set. The specificity refers to an accuracy of identifying non-cancer samples as the non-cancer label. The analytics system also calculates a sensitivity rate of prediction with the adjusted binary threshold cutoff against the holdout set. The sensitivity refers to an accuracy of identifying cancer samples as the cancer label. In practice, the specificity rate and/or the sensitivity rate may be defined according to a true positive rate, a false positive rate, a true negative rate, a false negative rate, another statistical calculation, etc.

The analytics system determines 1055 a TOO threshold for the TOO label. The analytics system selects the TOO threshold from the candidate TOO thresholds by optimizing the calculated specificity rates and/or sensitivity rates over the range of candidate TOO thresholds. In some examples, TOO thresholds are determined or otherwise applied for certain TOO tissue type classes or subtype classes, such as hematological classes. Merely by way of example, an algorithm for computing and applying TOO-specific probability thresholds can be used to remove non-cancer samples with exceeding signals of blood disorders. The algorithm can include, for each pre-specified TOO labels, first searching through a grid of probability values, and for every value, evaluating the clinical specificity and the clinical sensitivity of a holdout set using the binary detection threshold computed after removing non-cancer samples with equal or greater probability of the specified TOO label. By iterating through the probability grids, the algorithm will identify a combination of TOO threshold values for the pre-specified TOO labels that optimizes the tradeoff between the clinical specificity and the clinical sensitivity of the holdout set. The final optimized TOO probability threshold values will be used to filter out non-cancer samples that exceeds any of the values given the TOO labels. The cleaned set of non-cancer samples will be used to compute cancer-non-cancer detection threshold. Still, in some examples, the TOO-specific thresholding can be manually set at any cutpoint, such as a desired specificity level (e.g., 99.4-99.9% specificity).

The analytics system tunes 1065 the binary cancer classification by pruning non-cancer training samples exceeding the TOO thresholding prior to determining the binary threshold cutoff. The analytics system filters out non-cancer training samples from the training set according to the determined TOO threshold for the TOO label. The analytics system sets the binary threshold cutoff according to the filtered training set. For example, the analytics system determines a new binary threshold cutoff based on a filtered distribution of scores. In additional embodiments, the analytics system can determine a TOO threshold for any of the TOO labels according to steps 1010, 1020, 1030, and 1040, to tune the binary cancer classification.

### V.b. Stratification of Sample Distribution According to TOO Signal

In one or more embodiments, the analytics system tunes the cancer classifier by stratifying the sample distribution according to TOO signal to determine a binary threshold cutoff for each stratum. The analytics system may stratify the sample distribution according to the signal for one or more TOO labels, determined according a TOO prediction output by the multiclass cancer classifier.

As used herein, “high tissue signal” refers to a sample with a tissue signal, e.g., generally for any type of tissue or for a particular cancer type—also referred to as a TOO label, that exceeds some threshold. The tissue signal may be determined by a multiclass cancer classifier or other approaches, in comparison to a healthy distribution. Non-cancer samples with high tissue signal are outliers in the non-cancer distribution. Some of these non-cancer samples may be pre-stage cancer, early stage cancer, or undiagnosed cancer. The analytics system can identify non-cancer samples with high tissue signal in at least one TOO label. In one approach of determining high tissue signal, a prediction value for a TOO label output by the multiclass cancer classifier is compared against a tissue signal threshold. Samples with a prediction value above the tissue signal threshold are deemed to have high tissue signal for that TOO label; whereas, samples with a prediction value below the tissue signal threshold are deemed to not have high tissue signal for that TOO label (or low tissue signal). In another approach, one or more top predictions in a TOO prediction are considered. For example, a TOO prediction for a sample has a first prediction of the colorectal TOO label, a second prediction of the breast TOO label, and a third prediction of head/neck TOO label. If the top prediction is considered, then the sample is deemed to have high tissue signal for the TOO label in the first prediction, that being the colorectal TOO label in the example. If the top two predictions are considered, then there is high tissue signal in both the colorectal TOO label and the breast TOO label. Other approaches of determining tissue signal may include other models trained to determine tissue signal for one or more TOO labels. Such models may include classifiers trained to determine tissue signal for a subset of TOO labels. For example, a hematological-specific classifier may be trained and used to determine tissue signal for one or more hematological sub-types. Other models include deconvolution models that can deconvolve tissue signal from methylation sequencing data (and/or other types of sequencing data).

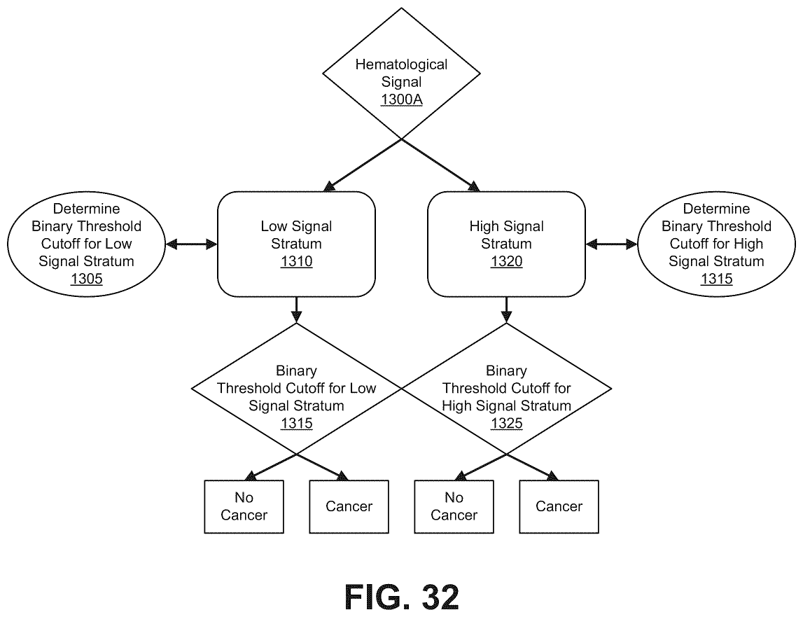

Referring now to FIG. 32, FIG. 32 illustrates a process for stratifying hematological signals into two strata, in accordance with one or more embodiments. Although the following description describes stratification with a hematological signal, the principles may be readily applied to other TOO signals.

The analytics system stratifies 1300A a holdout set of cancer and non-cancer samples according to the hematological signal into a low signal stratum 1310 and a high signal stratum 1320. Each sample of the holdout set has a cancer score determined by a binary cancer classifier and a TOO prediction determined by a multiclass cancer classifier. In one embodiment, hematological signal for a sample is determined according to a TOO prediction output by a multiclass cancer classifier. In one embodiment, when considering one or more top predictions (e.g., top one, top two, etc.), high hematological signal is determined if at least one of the top predictions being considered is one of a hematological sub-type (e.g., lymphoid neoplasm sub-type and myeloid neoplasm sub-type). Other hematological sub-types may be included. As such, if a sample has a TOO prediction with at least one of the top predictions being considered as the lymphoid neoplasm sub-type or the myeloid neoplasm sub-type, then the sample is determined to have high hematological signal. Otherwise, the sample is determined not to have high hematological signal.

The analytics system determines a binary threshold cutoff for each stratum for predicting presence or absence of cancer of a sample. The samples in the low signal stratum 1310 are used by the analytics system to determine 1305 a binary threshold cutoff for predicting absence or presence of cancer in samples in the low signal stratum 1310. The binary threshold cutoff is determined 1305 according to a false positive budget set for the low signal stratum 1310. With cancer scores for the samples in the low signal stratum 1310, the analytics system sweeps through a range of candidate binary threshold cutoffs evaluating a true positive rate (also referred to as sensitivity) and a false positive rate at each candidate binary threshold cutoff. The candidate binary threshold cutoff with a false positive rate that is closest within the false positive budget is determined to be the candidate binary threshold cutoff. The analytics system performs similar operations to determine 1315 a binary threshold cutoff for the high signal stratum 1320. The false positive budget for the low signal stratum 1310 and the false positive budget for the high signal stratum 1320 may be set according to a ratio of statistical true positive rates of the strata. The ratio aims to suppress the false positive rate in the high signal stratum 1320.

For a test sample, the analytics system places the test sample into either the low signal stratum 1310 or the high signal stratum 1320 according to hematological signal. If the test sample is placed in the low signal stratum 1310, then the analytics system applies 1315 the binary threshold cutoff for the low signal stratum 1310 to the cancer score of the test sample. If the cancer score is greater than or equal to the binary threshold cutoff for the low signal stratum 1310, then the analytics system returns a prediction of cancer presence in the test sample, and returns a prediction of no cancer otherwise. If test sample is placed in the high signal stratum 1320, then the binary threshold cutoff for the low signal stratum 1320 is applied 1325 to the cancer score of the test sample. If the cancer score is greater than or equal to the binary threshold cutoff for the high signal stratum 1320, then the analytics system returns a prediction of cancer presence in the test sample, and returns a prediction of no cancer otherwise.

### VI. CIRCULATING CELL-FREE GENOME ATLAS STUDY

In various embodiments, each predictive cancer model is trained using a set of training data derived from a training subset of patients of a circulating cell-free genome atlas (CCGA) study (See Clinical Trial.gov Identifier: NCT02889978 (https://www.clinicaltrials.gov/ct2/show/NCT02889978)) and then subsequently tested using a set of testing or validation data derived from a testing or validation subset of patients from the CCGA study.

The predictive cancer models described herein were trained using a plurality of known cancer types from the circulating cell-free genome atlas (CCGA) study. The CCGA sample set included the following cancer types: breast, lung, prostate, colorectal, renal, uterine, pancreas, esophageal, lymphoma, head and neck, ovarian, hepatobiliary, melanoma, cervical, multiple myeloma, leukemia, thyroid, bladder, gastric, and anorectal. As such, a model can be a multi-cancer model (or a multi-cancer classifier) for detecting of one or more, two or more, three or more, four or more, five or more, ten or more, or 20 or more different types of cancer. Predictive cancer models can be trained using a refined set of training data derived from a first subset of patients of the CCGA study and then subsequently tested using a refined set of testing data derived from a second subset of patients from the CCGA study.

### VII. CANCER ASSAY PANEL

In various embodiments, the predictive cancer models described herein use samples enriched using a cancer assay panel comprising a plurality of probes or a plurality of probe pairs. A number of targeted cancer assay panels are known in the art, for example, as describe in WO 2019/195268 filed Apr. 2, 2019, PCT/US2019/053509 filed Sep. 27, 2019 and PCT/US2020/015082 filed Jan. 24, 2020 (which are incorporated herein by reference). For example, in some embodiments, the cancer assay panel can be designed to include a plurality of probes (or probe pairs) that can capture fragments that can together provide information relevant to diagnosis of cancer. In some embodiments, a panel includes at least 50, 100, 500, 1,000, 2,000, 2,500, 5,000, 6,000, 7,500, 10,000, 15,000, 20,000, 25,000, or 50,000 pairs of probes. In other embodiments, a panel includes at least 500, 1,000, 2,000, 5,000, 10,000, 12,000, 15,000, 20,000, 30,000, 40,000, 50,000, or 100,000 probes. The plurality of probes together can comprise at least 0.1 million, 0.2 million, 0.4 million, 0.6 million, 0.8 million, 1 million, 2 million, 3 million, 4 million, 5 million, 6 million, 7 million, 8 million, 9 million, or 10 million nucleotides. The probes (or probe pairs) are specifically designed to target one or more genomic regions differentially methylated in cancer and non-cancer samples. The target genomic regions can be selected to maximize classification accuracy, subject to a size budget (which is determined by sequencing budget and desired depth of sequencing).

Samples enriched using a cancer assay panel can be subject to targeted sequencing. Samples enriched using the cancer assay panel can be used to detect the presence or absence of cancer generally and/or provide a cancer classification such as cancer type, stage of cancer such as I, II, III, or IV, or provide the tissue of origin where the cancer is believed to originate. Depending on the purpose, a panel can include probes (or probe pairs) targeting genomic regions differentially methylated between general cancerous (pan-cancer) samples and non-cancerous samples, or only in cancerous samples with a specific cancer type (e.g., lung cancer-specific targets). Specifically, a cancer assay panel is designed based on bisulfite sequencing data generated from the cell-free DNA (cfDNA) or genomic DNA (gDNA) from cancer and/or non-cancer individuals.

In some embodiments, the cancer assay panel designed by methods provided herein comprises at least 1,000 pairs of probes, each pair of which comprises two probes configured to overlap each other by an overlapping sequence comprising a 30-nucleotide fragment. The 30-nucleotide fragment comprises at least five CpG sites, wherein at least 80% of the at least five CpG sites are either CpG or UpG. The 30-nucleotide fragment is configured to bind to one or more genomic regions in cancerous samples, wherein the one or more genomic regions have at least five methylation sites with an abnormal methylation pattern. Another cancer assay panel comprises at least 2,000 probes, each of which is designed as a hybridization probe complimentary to one or more genomic regions. Each of the genomic regions is selected based on the criteria that it comprises (i) at least 30 nucleotides, and (ii) at least five methylation sites, wherein the at least five methylation sites have an abnormal methylation pattern and are either hypomethylated or hypermethylated.

Each of the probes (or probe pairs) is designed to target one or more target genomic regions. The target genomic regions are selected based on several criteria designed to increase selective enriching of relevant cfDNA fragments while decreasing noise and non-specific bindings. For example, a panel can include probes that can selectively bind and enrich cfDNA fragments that are differentially methylated in cancerous samples. In this case, sequencing of the enriched fragments can provide information relevant to diagnosis of cancer. Furthermore, the probes can be designed to target genomic regions that are determined to have an abnormal methylation pattern and/or hypermethylation or hypomethylation patterns to provide additional selectivity and specificity of the detection. For example, genomic regions can be selected when the genomic regions have a methylation pattern with a low p-value according to a Markov model trained on a set of non-cancerous samples, that additionally cover at least 5 CpG's, 90% of which are either methylated or unmethylated. In other embodiments, genomic regions can be selected utilizing mixture models, as described herein.

Each of the probes (or probe pairs) can target genomic regions comprising at least 25 bp, 30 bp, 35 bp, 40 bp, 45 bp, 50 bp, 60 bp, 70 bp, 80 bp, or 90 bp. The genomic regions can be selected by containing less than 20, 15, 10, 8, or 6 methylation sites. The genomic regions can be selected when at least 80, 85, 90, 92, 95, or 98% of the at least five methylation (e.g., CpG) sites are either methylated or unmethylated in non-cancerous or cancerous samples.

Genomic regions may be further filtered to select only those that are likely to be informative based on their methylation patterns, for example, CpG sites that are differentially methylated between cancerous and non-cancerous samples (e.g., abnormally methylated or unmethylated in cancer versus non-cancer). For the selection, calculation can be performed with respect to each CpG site. In some embodiments, a first count is determined that is the number of cancer-containing samples (cancer_count) that include a fragment overlapping that CpG, and a second count is determined that is the number of total samples containing fragments overlapping that CpG (total). Genomic regions can be selected based on criteria positively correlated to the number of cancer-containing samples (cancer_count) that include a fragment overlapping that CpG, and inversely correlated with the number of total samples containing fragments overlapping that CpG (total).

In one embodiment, the number of non-cancerous samples (n_{non-cancer}) and the number of cancerous samples (n_{cancer}) having a fragment overlapping a CpG site are counted. Then the probability that a sample is cancer is estimated, for example as (n_{cancer}+1)/(n_{cancer}+n_{non-cancer}+2). CpG sites by this metric are ranked and greedily added to a panel until the panel size budget is exhausted.

Depending on whether the assay is intended to be a pan-cancer assay or a single-cancer assay, or depending on what kind of flexibility is desired when picking which CpG sites are contributing to the panel, which samples are used for cancer-count can vary. A panel for diagnosing a specific cancer type (e.g., TOO) can be designed using a similar process. In this embodiment, for each cancer type, and for each CpG site, the information gain is computed to determine whether to include a probe targeting that CpG site. The information gain is computed for samples with a given cancer type compared to all other samples. For example, two random variables, “AF” and “CT”. “AF” is a binary variable that indicates whether there is an abnormal fragment overlapping a particular CpG site in a particular sample (yes or no). “CT” is a binary random variable indicating whether the cancer is of a particular type (e.g., lung cancer or cancer other than lung). One can compute the mutual information with respect to “CT” given “AF.” That is, how many bits of information about the cancer type (lung vs. non-lung in the example) are gained if one knows whether there is an informative fragment overlapping a particular CpG site. This can be used to rank CpG's based on how specific they are for a particular cancer type (e.g., TOO). This procedure is repeated for a plurality of cancer types. For example, if a particular region is commonly differentially methylated only in lung cancer (and not other cancer types or non-cancer), CpG's in that region would tend to have high information gains for lung cancer. For each cancer type, CpG sites ranked by this information gain metric, and then greedily added to a panel until the size budget for that cancer type was exhausted.

Further filtration can be performed to select target genomic regions that have off-target genomic regions less than a threshold value. For example, a genomic region is selected only when there are less than 15, 10 or 8 off-target genomic regions. In other cases, filtration is performed to remove genomic regions when the sequence of the target genomic regions appears more than 5, 10, 15, 20, 25, or 30 times in a genome. Further filtration can be performed to select target genomic regions when a sequence, 90%, 95%, 98% or 99% homologous to the target genomic regions, appear less than 15, 10 or 8 times in a genome, or to remove target genomic regions when the sequence, 90%, 95%, 98% or 99% homologous to the target genomic regions, appear more than 5, 10, 15, 20, 25, or 30 times in a genome. This is for excluding repetitive probes that can pull down off-target fragments, which are not desired and can impact assay efficiency.

In some embodiments, fragment-probe overlap of at least 45 bp was demonstrated to be required to achieve a non-negligible amount of pulldown (though this number can be different depending on assay details). Furthermore, it has been suggested that more than a 10% mismatch rate between the probe and fragment sequences in the region of overlap is sufficient to greatly disrupt binding, and thus pulldown efficiency. Therefore, sequences that can align to the probe along at least 45 bp with at least a 90% match rate are candidates for off-target pulldown. Thus, in one embodiment, the number of such regions are scored. The best probes have a score of 1, meaning they match in only one place (the intended target region). Probes with a low score (say, less than 5 or 10) are accepted, but any probes above the score are discarded. Other cutoff values can be used for specific samples.

In various embodiments, the selected target genomic regions can be located in various positions in a genome, including but not limited to exons, introns, intergenic regions, and other parts. In some embodiments, probes targeting non-human genomic regions, such as those targeting viral genomic regions, can be added.

### VIII. KIT IMPLEMENTATION

Also disclosed herein are kits for performing the methods described above including the methods relating to the cancer classifier. The kits may include one or more collection vessels for collecting a sample from the individual comprising genetic material. The sample can include blood, plasma, serum, urine, fecal, saliva, other types of bodily fluids, or any combination thereof. Such kits can include reagents for isolating nucleic acids from the sample. The reagents can further include reagents for sequencing the nucleic acids including buffers and detection agents. In one or more embodiments, the kits may include one or more sequencing panels comprising probes for targeting particular genomic regions, particular mutations, particular genetic variants, or some combination thereof. In one or more embodiments, the kit comprises at least one panel comprising contamination targeting probes. In other embodiments, samples collected via the kit are provided to a sequencing laboratory that may use the sequencing panels to sequence the nucleic acids in the sample.

A kit can further include instructions for use of the reagents included in the kit. For example, a kit can include instructions for collecting the sample, extracting the nucleic acid from the test sample. Example instructions can be the order in which reagents are to be added, centrifugal speeds to be used to isolate nucleic acids from the test sample, how to amplify nucleic acids, how to sequence nucleic acids, or any combination thereof. The instructions may further illumine how to operate a computing device as the analytics system 200, for the purposes of performing the steps of any of the methods described.

In addition to the above components, the kit may include computer-readable storage media storing computer software for performing the various methods described throughout the disclosure. One form in which these instructions can be present is as printed information on a suitable medium or substrate, e.g., a piece or pieces of paper on which the information is printed, in the packaging of the kit, in a package insert. Yet another means would be a computer readable medium, e.g., diskette, CD, hard-drive, network data storage, on which the instructions have been stored in the form of computer code. Yet another means that can be present is a website address which can be used via the internet to access the information at a removed site.

### IX. CANCER APPLICATIONS

In some embodiments, the methods, analytic systems and/or classifier of the present disclosure can be used to detect the presence (or absence) of cancer, monitor cancer progression or recurrence, monitor therapeutic response or effectiveness, determine a presence or monitor minimum residual disease (MRD), or any combination thereof. In some embodiments, the analytic systems and/or classifier may be used to identify the tissue or origin for a cancer. For instance, the systems and/or classifiers may be used to identify a cancer as of any of the following cancer types: head and neck cancer, liver/bileduct cancer, upper GI cancer, pancreatic/gallbladder cancer; colorectal cancer, ovarian cancer, lung cancer, multiple myeloma, lymphoid neoplasms, melanoma, sarcoma, breast cancer, and uterine cancer. For example, as described herein, a classifier can be used to generate a likelihood or probability score (e.g., from 0 to 100) that a sample feature vector is from a subject with cancer. In some embodiments, the probability score is compared to a threshold probability to determine whether or not the subject has cancer. In other embodiments, the likelihood or probability score can be assessed at different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the likelihood or probability score exceeds a threshold, a physician can prescribe an appropriate treatment. In some embodiments, a test report can be generated to provide a patient with their test results, including, for example, a probability score that the patient has a disease state (e.g., cancer), a type of disease (e.g., a type of cancer), and/or a disease tissue of origin (e.g., a cancer tissue of origin).

### IX.a. Early Detection of Cancer

In some embodiments, the methods and/or classifier of the present disclosure are used to detect the presence or absence of cancer in a subject suspected of having cancer. For example, a classifier (as described herein) can be used to determine a likelihood or probability score that a sample feature vector is from a subject that has cancer.

In one embodiment, a probability score of greater than or equal to 60 can indicated that the subject has cancer. In still other embodiments, a probability score greater than or equal to 65, greater than or equal to 70, greater than or equal to 75, greater than or equal to 80, greater than or equal to 85, greater than or equal to 90, or greater than or equal to 95, indicated that the subject has cancer. In other embodiments, a probability score can indicate the severity of disease. For example, a probability score of 80 may indicate a more severe form, or later stage, of cancer compared to a score below 80 (e.g., a score of 70). Similarly, an increase in the probability score over time (e.g., at a second, later time point) can indicate disease progression or a decrease in the probability score over time (e.g., at a second, later time point) can indicate successful treatment.

In another embodiment, a cancer log-odds ratio can be calculated for a test subject by taking the log of a ratio of a probability of being cancerous over a probability of being non-cancerous (i.e., one minus the probability of being cancerous), as described herein. In accordance with this embodiment, a cancer log-odds ratio greater than 1 can indicate that the subject has cancer. In still other embodiments, a cancer log-odds ratio greater than 1.2, greater than 1.3, greater than 1.4, greater than 1.5, greater than 1.7, greater than 2, greater than 2.5, greater than 3, greater than 3.5, or greater than 4, indicated that the subject has cancer. In other embodiments, a cancer log-odds ratio can indicate the severity of disease. For example, a cancer log-odds ratio greater than 2 may indicate a more severe form, or later stage, of cancer compared to a score below 2 (e.g., a score of 1). Similarly, an increase in the cancer log-odds ratio over time (e.g., at a second, later time point) can indicate disease progression or a decrease in the cancer log-odds ratio over time (e.g., at a second, later time point) can indicate successful treatment.

According to aspects of the disclosure, the methods and systems of the present disclosure can be trained to detect or classify multiple cancer indications. For example, the methods, systems and classifiers of the present disclosure can be used to detect the presence of one or more, two or more, three or more, five or more, or ten or more different types of cancer.

In some embodiments, the cancer is one or more of head and neck cancer, liver/bileduct cancer, upper GI cancer, pancreatic/gallbladder cancer; colorectal cancer, ovarian cancer, lung cancer, multiple myeloma, lymphoid neoplasms, melanoma, sarcoma, breast cancer, and uterine cancer.

### IX.b. Cancer and Treatment Monitoring

In certain embodiments, the first time point is before a cancer treatment (e.g., before a resection surgery or a therapeutic intervention), and the second time point is after a cancer treatment (e.g., after a resection surgery or therapeutic intervention), and the method utilized to monitor the effectiveness of the treatment. For example, if the second likelihood or probability score decreases compared to the first likelihood or probability score, then the treatment is considered to have been successful. However, if the second likelihood or probability score increases compared to the first likelihood or probability score, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., before a resection surgery or a therapeutic intervention) and the method is used to monitor the effectiveness of the treatment or loss of effectiveness of the treatment. In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the disclosure to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 3 months, at least once every 6 months, at least once a year, at least once every 2 years, at least once every 3 years, at least once every 4 years, or at least once every 5 years.

### IX.c. Treatment

In still another embodiment, information obtained from any method described herein (e.g., the likelihood or probability score) can be used to make or influence a clinical decision (e.g., diagnosis of cancer, treatment selection, assessment of treatment effectiveness, etc.). For example, in one embodiment, if the likelihood or probability score exceeds a threshold, a physician can prescribe an appropriate treatment (e.g., a resection surgery, radiation therapy, chemotherapy, and/or immunotherapy). In some embodiments, information such as a likelihood or probability score can be provided as a readout to a physician or subject.

A classifier (as described herein) can be used to determine a likelihood or probability score that a sample feature vector is from a subject that has cancer. In one embodiment, an appropriate treatment (e.g., resection surgery or therapeutic) is prescribed when the likelihood or probability exceeds a threshold. For example, in one embodiment, if the likelihood or probability score is greater than or equal to 60, one or more appropriate treatments are prescribed. In another embodiments, if the likelihood or probability score is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, a cancer log-odds ratio can indicate the effectiveness of a cancer treatment. For example, an increase in the cancer log-odds ratio over time (e.g., at a second, after treatment) can indicate that the treatment was not effective. Similarly, a decrease in the cancer log-odds ratio over time (e.g., at a second, after treatment) can indicate successful treatment. In another embodiment, if the cancer log-odds ratio is greater than 1, greater than 1.5, greater than 2, greater than 2.5, greater than 3, greater than 3.5, or greater than 4, one or more appropriate treatments are prescribed.

In some embodiments, the treatment is one or more cancer therapeutic agents selected from the group including a chemotherapy agent, a targeted cancer therapy agent, a differentiating therapy agent, a hormone therapy agent, and an immunotherapy agent. For example, the treatment can be one or more chemotherapy agents selected from the group including alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group including signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group including anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### X. EXAMPLES

### X.a. Example 1—Whole-Genome Bisulfite Sequencing (WBGS)

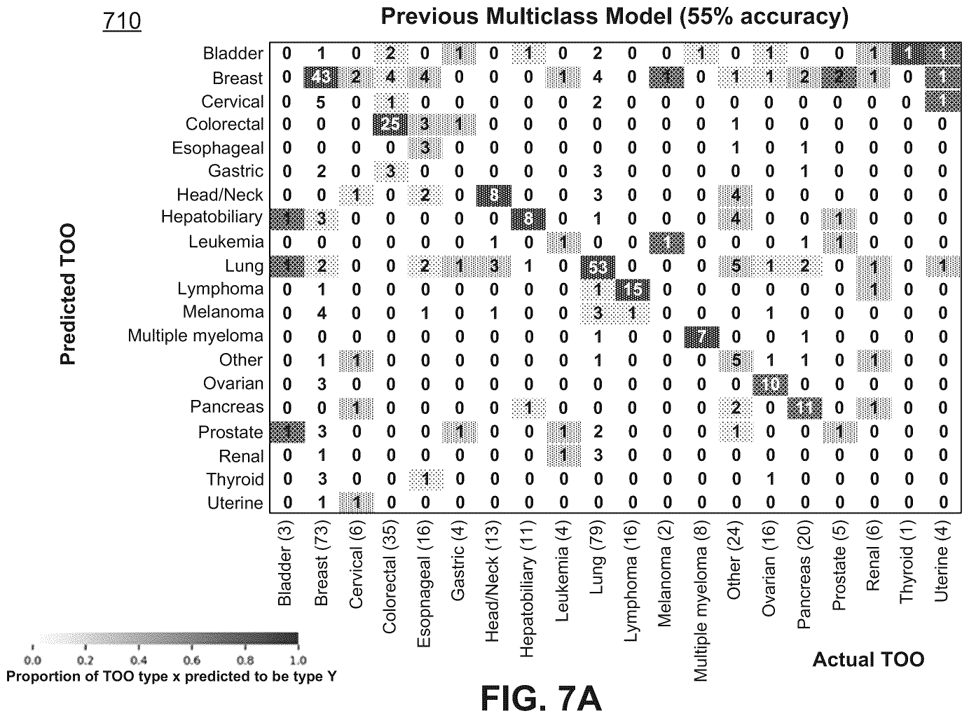

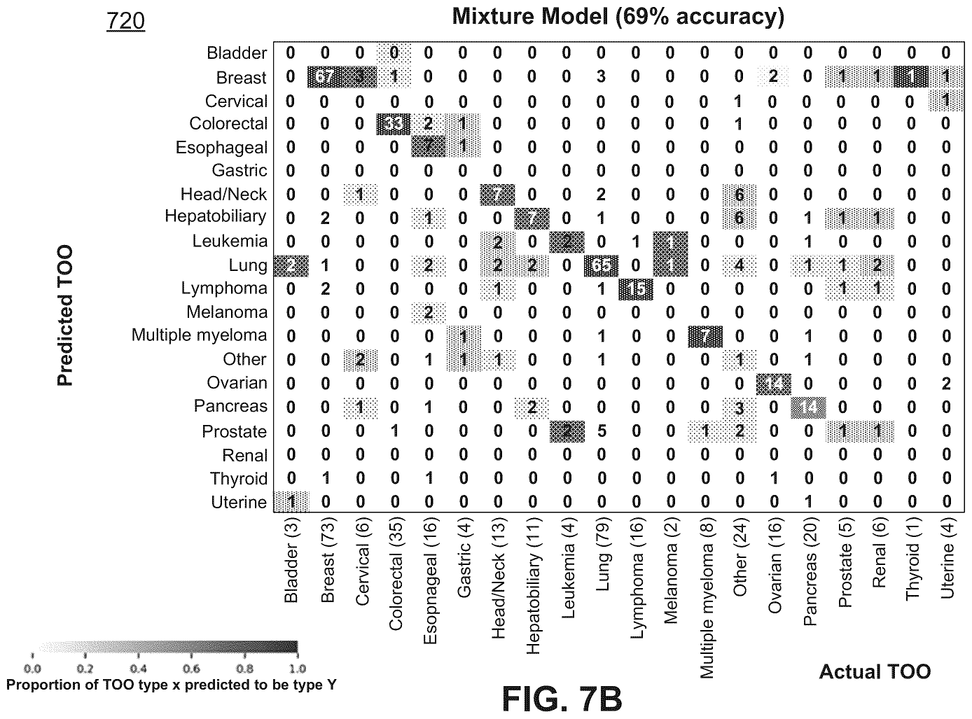

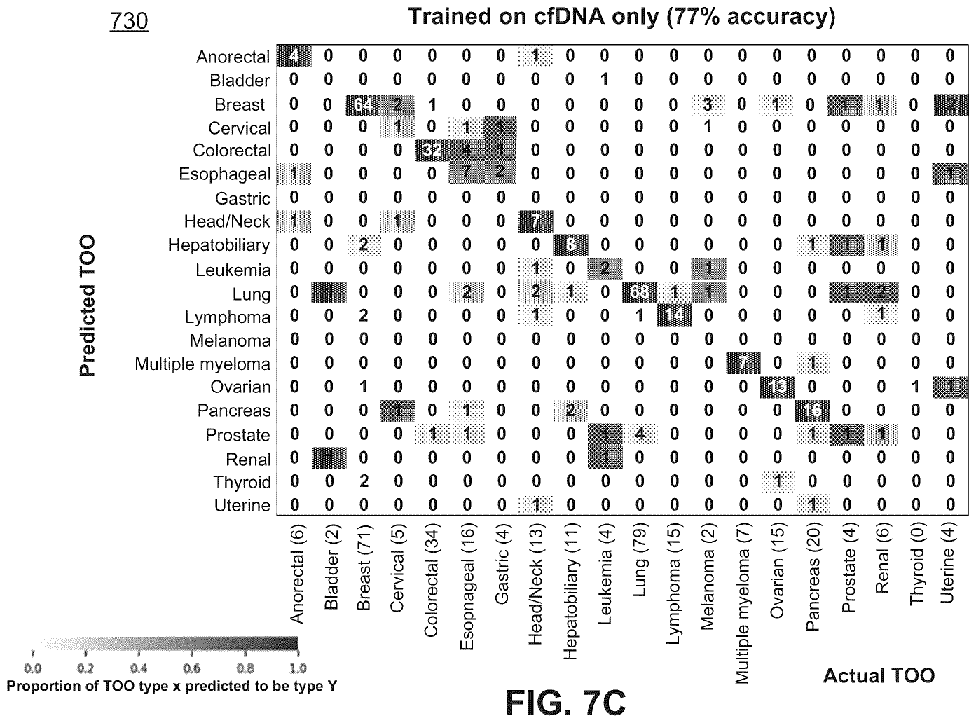

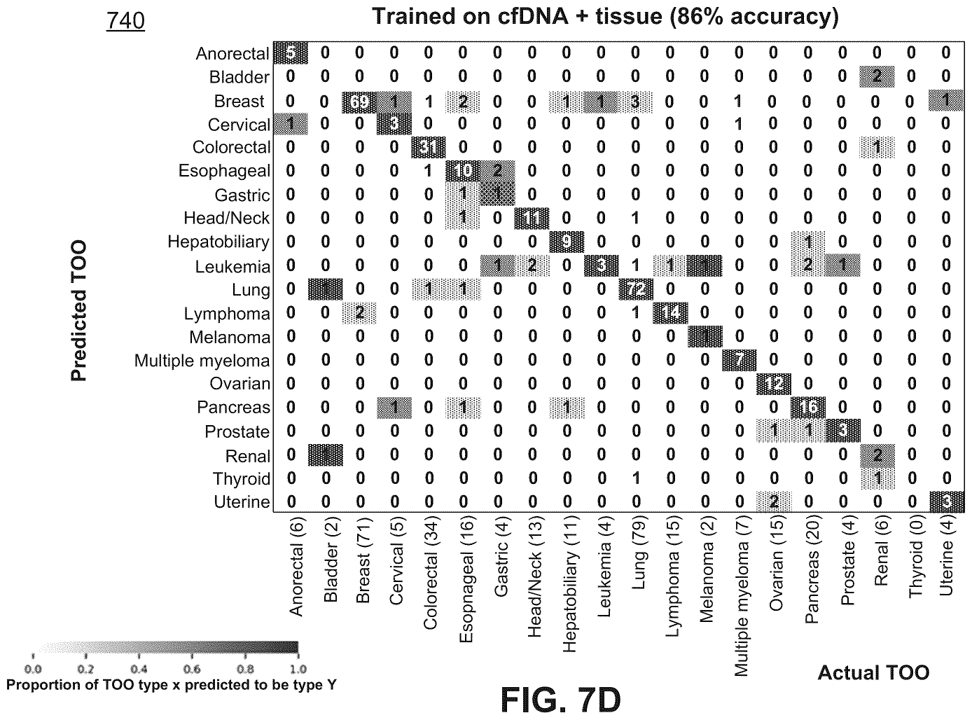

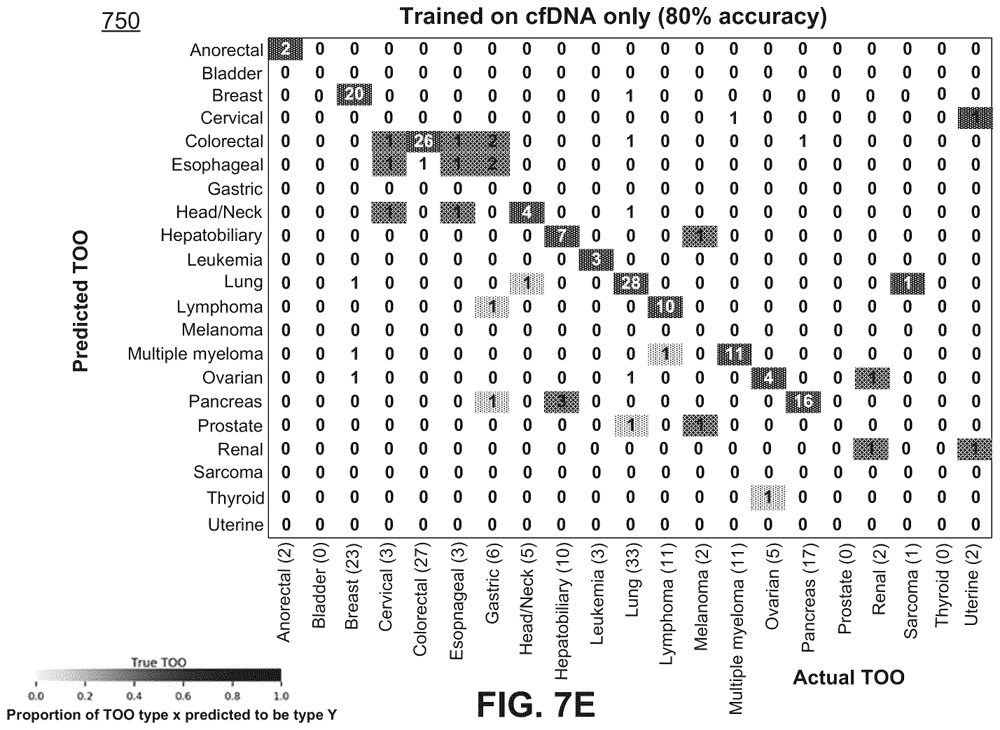

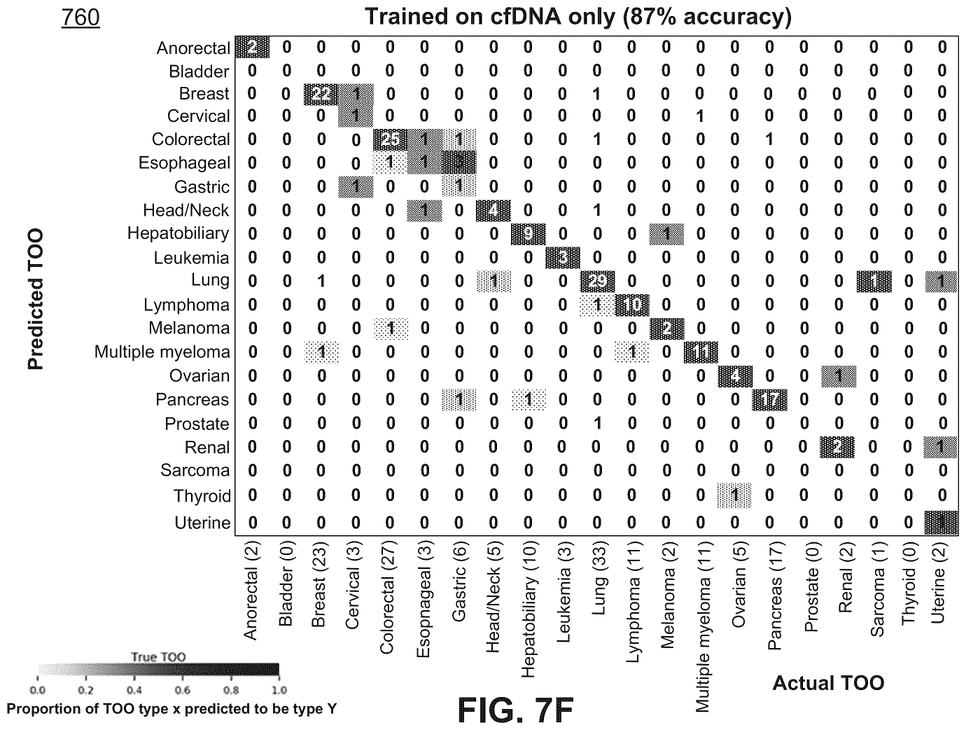

First CCGA substudy: The data shown in FIGS. 7A-F were obtained from a first CCGA substudy where training data blood samples (N=1785) were collected from individuals diagnosed with untreated cancer (including 20 tumor types and all stages of cancer) and healthy individuals with no cancer diagnosis (controls) for plasma cfDNA extraction. Another set of blood samples (N=1,010) were collected to be used for validation. Unless otherwise indicated, extracted cell-free DNA (cfDNA) and genomic DNA (gDNA) from the first CCGA substudy samples were subjected to a whole-genome bisulfite sequencing assay.

In the classification process, the analytics system 200 treats fragment methylation states as being drawn from a mixture of latent methylation patterns. The analytics system 200 assigns observed fragments a relative probability of originating from a particular cancer tissue of origin.

More specifically, as described herein, a probabilistic model was fit to the sequence reads derived from a plurality of regions (or windows) from each cancer type (and for non-cancer or healthy samples). In this case, a mixture model was used where each mixture component was an independent-sites model (in which methylation at each CpG is independent of methylation at other CpGs). Models were fit using maximum likelihood estimation to identify the set of parameters that maximize the total log-likelihood of all fragments derived from one cancer type (or non-cancer).

For each region, for each cancer type pair (including non-cancer as a negative type), the best performing tiers were used to train a multinomial logistic regression classifier. For each sample (regardless of label), in each region, for each cancer type, for each fragment, the log-likelihood ratio was calculated, as previously described, and for each of a set of “tier” values the number of fragments with R_{cancer, type}>tier were quantified. Quantified reads for each of the tiers were binarized and used as features to train the classifier.

Finally, where indicated, to generate predictions for an unknown sample feature values were determined (as described above) and the generated features were used to create a cancer and/or tissue of origin prediction utilizing the trained multinomial logistic regression classifier.

Example confusion matrices: FIGS. 7A, 7B, and 7C include confusion matrices indicating accuracy of classifiers, according to various embodiments. In some embodiments, the analytics system 200 determines an accuracy of the classifier using a confusion matrix. The confusion matrix includes information describing a success rate for the classifier at identifying each of the disease states.

As shown in FIG. 7A, matrix 710 includes example performance of a classifier based on a multinomial model trained using a set of cfDNA samples (no tissue samples). Matrix 720 includes an example performance of a classifier based on a mixture model trained by the analytics system 200 using the same set of cfDNA samples. Scores along the diagonal of the matrices indicate correct predictions, that is, where the predicted tissue of origin for a fragment matches the true tissue of origin. In comparison to the classifier based on the multinomial model as a baseline, the classifier based on the mixture model has greater overall accuracy in predicting presence of the types of cancers shown in the matrices.

Samples of the training sets can be filtered based on one or more criteria (e.g., a particular specificity level). For example, the training sets include samples determined to have cancer based on a 98% specificity according to an m-score. The remaining (e.g., 2%) non-cancer samples that were (erroneously) identified as having cancer were excluded from being displayed in the confusion matrices for clarity.

As shown in FIG. 7B, matrix 730 includes an example performance of a classifier based on a mixture model trained using a cross-validation training set of cfDNA samples (no tissue samples). Matrix 740 includes an example performance of a classifier based on a mixture model trained using a cross-validation training set of cfDNA and tissue samples.

As shown in FIG. 7C, matrix 750 includes an example performance of a classifier based on a mixture model trained using a set of cfDNA samples (no tissue samples) from a clinical study titled Circulating Cell-free Genome Atlas Study (“CCGA”). Matrix 740 includes an example performance of a classifier based on a mixture model trained using a set of cfDNA and tissue samples from CCGA. The CCGA study was described with Clinical Trial.gov Identifier: NCT02889978 (https://www.clinicaltrials.gov/ct2/show/NCT02889978).

### X.b. Example 2—Classification of Cancer Using Targeted Bisulfite Sequencing from Early Breakout of the Second CCGA Substudy

Second CCGA substudy: The data shown in FIGS. 9A-B, 10A-B, 11, and 12 were obtained from an early breakout from the second CCGA sub-study where training data blood samples (N=3,132) were collected from individuals diagnosed with untreated cancer (including 20 tumor types and all stages of cancer) and healthy individuals with no cancer diagnosis (controls) for plasma cfDNA extraction. Another set of blood samples (N=1,354) were collected to be used for validation. In some embodiments, where indicated, the training set also included training data from tissue samples (i.e., gDNA). To determine the analysis population, the training data blood samples were filtered based on several factors. For example, 105 samples were excluded as clinically unlocked; 11 samples were excluded based on eligibility criteria; 58 samples were excluded for unconfirmed cancer or treatment status (not evaluable); 4 non-processed samples and 72 non-evaluable assays were excluded (not analyzable); and 581 samples were reserved for future analysis. As a result, the analysis population of 2,301 samples included 1,422 cancer samples and 879 non-cancer samples.

Participant demographics of individuals in the sub-study are shown below in Table 1.

TABLE 1

| | Cancer* | Non-Cancer |
|---|---|---|
| Total | 1,422 | 879 |
| Age, Mean ± SD | 62.0 ± 11.8 | 54.2 ± 13.6 |
| Age Group, n (%) | | |
| ≥50 years | 1220 (85.8) | 576 (65.5) |
| Sex, n (%) | | |
| Female | 712 (50.1) | 583 (66.3) |
| Race/Ethnicity (%) | | |
| White, Non-Hispanic | 1174 (82.6) | 713 (81.1) |
| African American | 97 (6.8) | 67 (7.6) |
| Hispanic, Asian, Other | 151 (10.6) | 99 (11.3) |
| Smoking Status, n (%)^{†} | | |
| Never-smoker | 633 (45.3) | 495 (57.1) |
| Body Mass Index, n (%)^{‡} | | |
| Normal/Underweight | 381 (26.8) | 216 (24.6) |
| Overweight | 490 (34.5) | 309 (35.2) |
| Obese | 551 (38.7) | 352 (40.1) |
| Method of Dx, n (%) | | |
| Dx by Screening | 350 (24.6) | — |
| Clinical Stage, n (%)^{§} | | |
| I | 398 (28.0) | — |
| II | 366 (25.7) | — |
| III | 290 (20.4) | — |
| IV | 327 (23.0) | — |
| Non-informative/Missing^{¶} | 41 (2.9) | — |

Table 1:
Participant demographics and stage distribution. Cancer and non-cancer groups were comparable with respect to age, race, sex, and body mass index (not shown).
*Includes anorectal, bladder, brain, breast, cervical, colorectal, esophageal, gastric, head and neck, hepatobiliary, lung, lymphoid neoplasm (chronic lymphocytic leukemia, lymphoma), multiple myeloma, myeloid neoplasm (acute myeloid leukemia, chronic myeloid leukemia), ovarian, pancreatic, prostate, renal, sarcoma, and uterine cancers.
†Excludes 38 participants missing smoking status information.
‡Excludes two participants missing BMI values.
^{§}Invasive cancer only.
^{¶}Staging information not available.

To identify cancer-defining and tissue-defining methylation signals, the extracted cfDNA was subjected to a bisulfite sequencing assay targeting the most informative regions of the methylome, as identified from GRAIL's proprietary whole-genome bisulfite sequencing assay and methylation database.

We used a methylation database that interrogated genome-wide fragment-level methylation patters across 811 cancer cell methylomes representing 21 tumor types (97% of SEER cancer incidence). To generate the methylation database of cancer-defining methylation signals, genomic DNA from formalin-fixed, paraffin-embedded (FFPE) tumor tissues and isolated cells from tumors were subjected to a whole-genome bisulfite sequencing assay. The methylation database was used for panel design and training to optimize performance of classifiers, as described herein. A large methylation sequence database of cancer and non-cancer was generated to enable target selection for a single test able to classify multiple cancers at high specificity and identify tissue of origin.

Target selection and panel design: Target genomic regions were selected using the methylation sequence database from the CCGA study, as described herein. Specifically, cfDNA sequences in the database were filtered based on p-value using a non-cancer distribution, and only fragments with p<0.001 were retained. The selected cfDNAs were further filtered to retain only those that were at least 90% methylated or 90% unmethylated. Next, for each CpG site in the selected fragments, the numbers of cancer samples or non-cancer samples were counted that include fragments overlapping that CpG site. Specifically, P (cancer|overlapping fragment) for each CpG was calculated and genomic sites with high P values were selected as general cancer targets. By design, the selected fragments had very low noise (i.e., few non-cancer fragments overlapping).

To find cancer type specific targets, similar selection processes were performed. CpG sites were ranked based on their information gain, comparing one cancer type to all other samples (i.e., non-cancer plus other cancer types).

Cancer assay panels comprising probes targeting the selected genomic regions were generated, as described herein. Specifically, the panels were designed to detect the presence of cancer generally (i.e., vs non-cancer) or a specific cancer type (e.g., TOO). The panels include probe set targeting each of the genomic regions selected.

Probes were designed to overlap any of the CpG sites included within the start/stop ranges of any of the targeted regions (e.g., informative fragments).

Classification: In the classification process, the analytics system 200 treats fragment methylation states as being drawn from a mixture of latent methylation patterns. The analytics system 200 assigns observed fragments a relative probability of originating from cancer. For tissue of origin classification, the analytics system 200 assigns observed fragments a relative probability of originating from a particular tissue. The analytics system 200 combines fragments characteristic of cancer and tissue of origin across targeted regions to classify cancer versus non-cancer and/or identify tissue of origin. For binary cancer classification, the analytics system 200 estimates sensitivity at 99% specificity.

More specifically, as described in Example VI.a, a probabilistic model was fit to the sequence reads derived from a plurality of regions (or windows) from each cancer type (and for non-cancer or healthy samples), features identified, and a multinomial logistic regression classifier trained. To generate predictions for an unknown sample feature values were determined (as described above) and the generated features were used to create a cancer and/or tissue of origin prediction utilizing the trained multinomial logistic regression classifier.

FIGS. 9A and 9B illustrate sensitivity of tissue of origin classifiers generated by methods described in the present disclosure. The sensitivity is reported at 99% specificity, and 95% confidence intervals are indicated. FIG. 9A illustrates model predictions for a pre-specified list of cancers. FIG. 9B illustrates model predictions for other cancers included in the CCGA study. Demographic information alone (baseline modeling) classified<5% of participants correctly. Overall sensitivity was 76.1% (95% CI: 73.1-78.9%) in a pre-specified list of cancers (anorectal, breast [HR-negative], colorectal, esophageal, gastric, head and neck, hepatobiliary, lung, lymphoid neoplasm [chronic lymphocytic leukemia, lymphoma], multiple myeloma, ovarian, pancreatic). Sensitivity was 68.8% (95% CI: 64.8-72.6%) in early stage (I-III) cancers in this cohort. Overall sensitivity was 55.1% (95% CI: 52.5-57.7%) across all cancer types and stages. In early stage (I-III) cancers, sensitivity was 43.8% (95% CI: 40.7-46.8%).

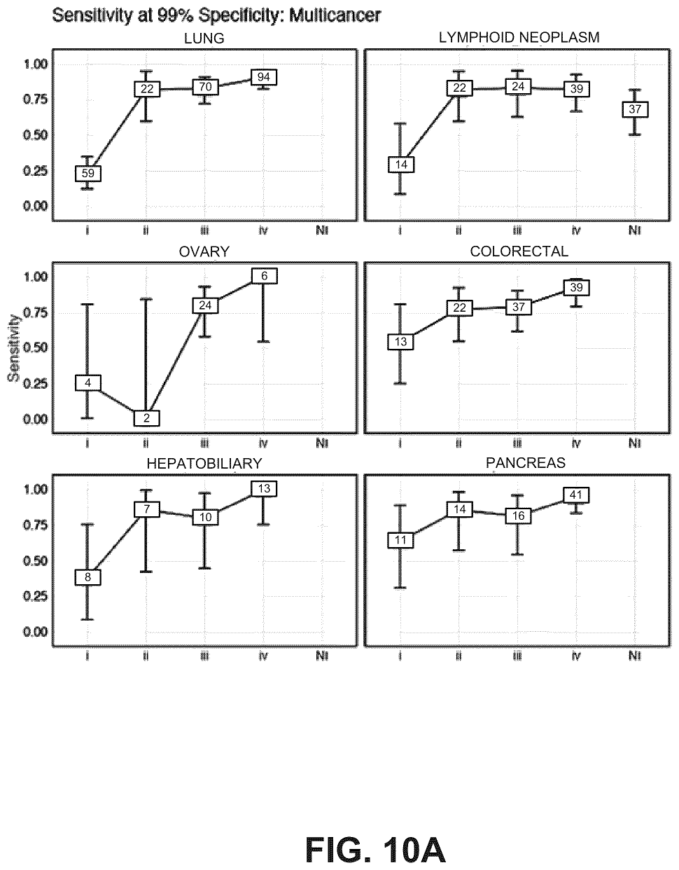

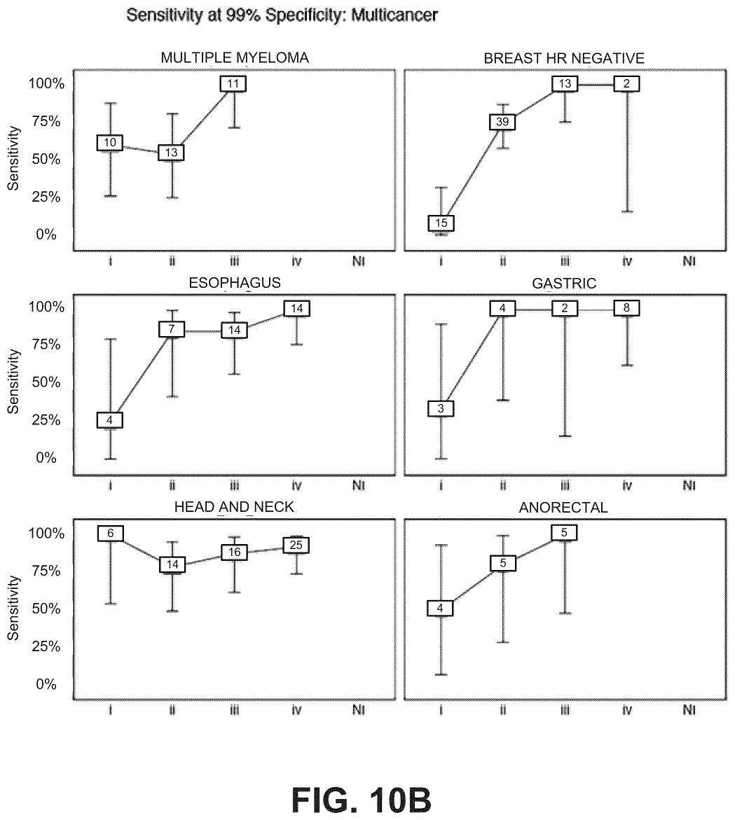

FIGS. 10A and 10B illustrate sensitivity of the tissue of origin classifiers at different cancer stages. Sensitivity by individual stage, as indicated in the legend, for the pre-specified cancers-of-interest in aggregate is reported at 99% specificity. Numbers within boxes represent the total number of samples included at each stage. 95% confidence intervals are indicated. “Lymphoid neoplasm” includes lymphoma (stages I-IV) and chronic lymphocytic leukemia (un-staged, included as “NI”).

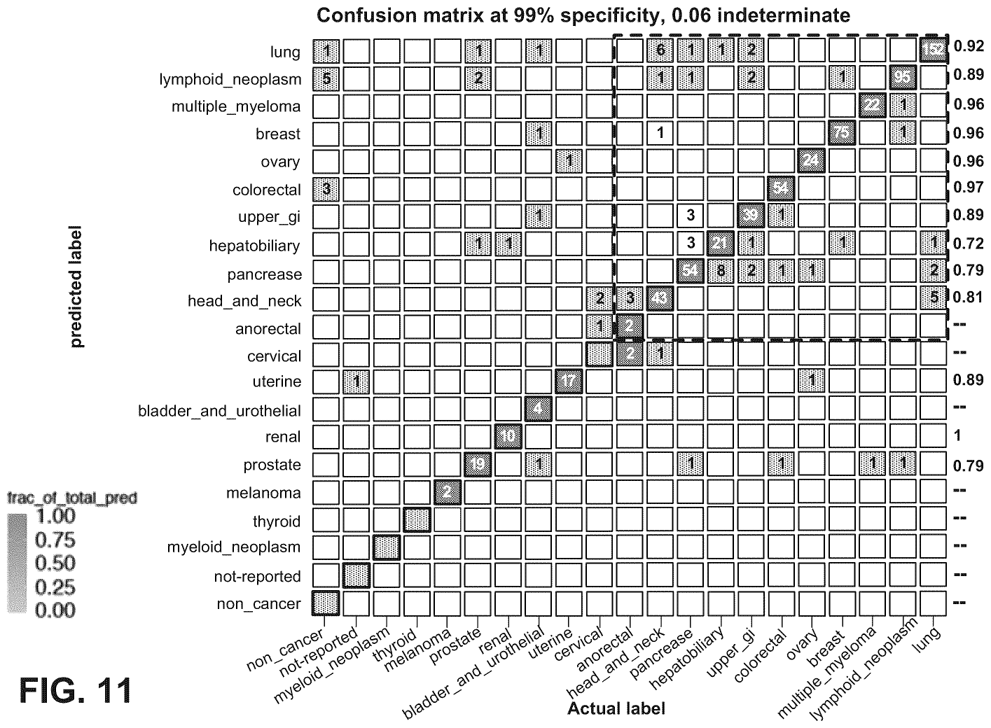

FIG. 11 illustrates a performance grid representing the accuracy of tissue of origin localization. There is agreement between the true (x-axis) and predicted (y-axis) tissue of origin per sample using the tissue of origin classifier with the methylation database in stage I-IV samples. The gradient legend corresponds to the proportion of predicted tissue of origin (y-axis) which were correct (x-axis). The analysis showed that accuracy of tissue of origin localization (the fraction of all TOO predictions that were correct) was higher with the methylation database (p=0.0066). This was consistent in stage I-III predictions: 89.9% (384/427) as further demonstrated in Table 2.

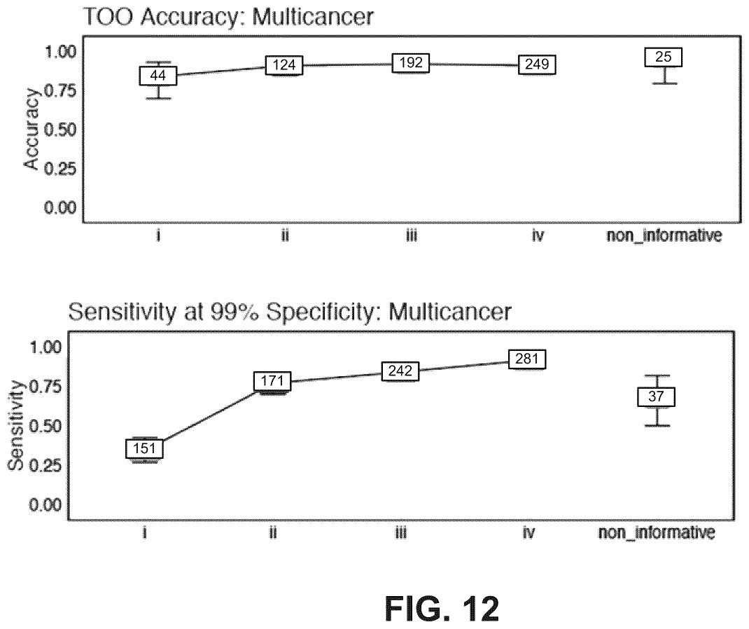

An effective multi-cancer test ideally should simultaneously detect clinically significant cancers across stages with very high specificity (and thus would have a single fixed, low false positive rate), and accurately determine tissue of origin. To demonstrate the potential of this approach, simultaneous detection (sensitivity reported at 99% specificity) and tissue of origin determination for the pre-specified list of cancer types, in aggregate, at individual stages, is displayed in FIG. 12. Thus, FIG. 12 illustrates accuracy and sensitivity of a tissue of origin classifier at different cancer stages

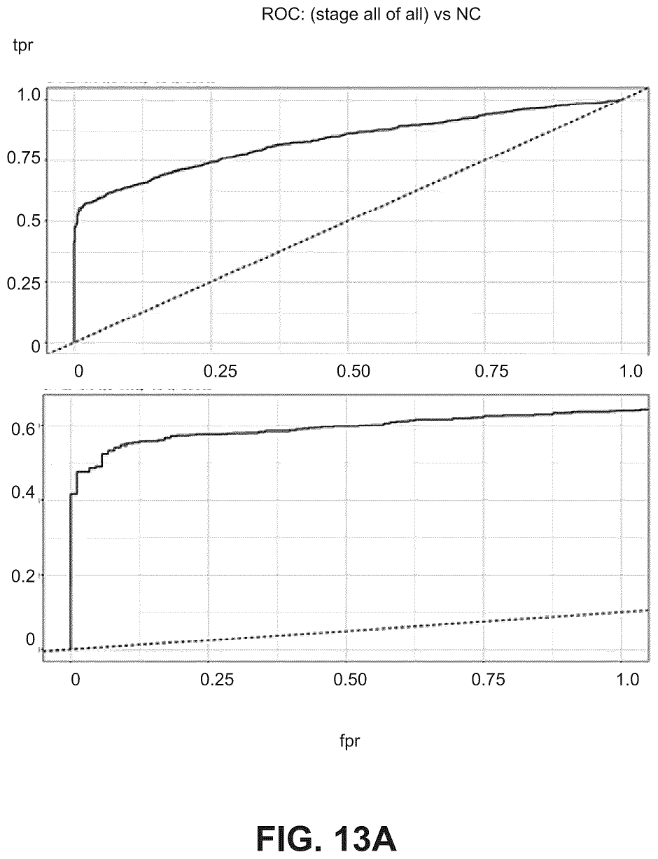

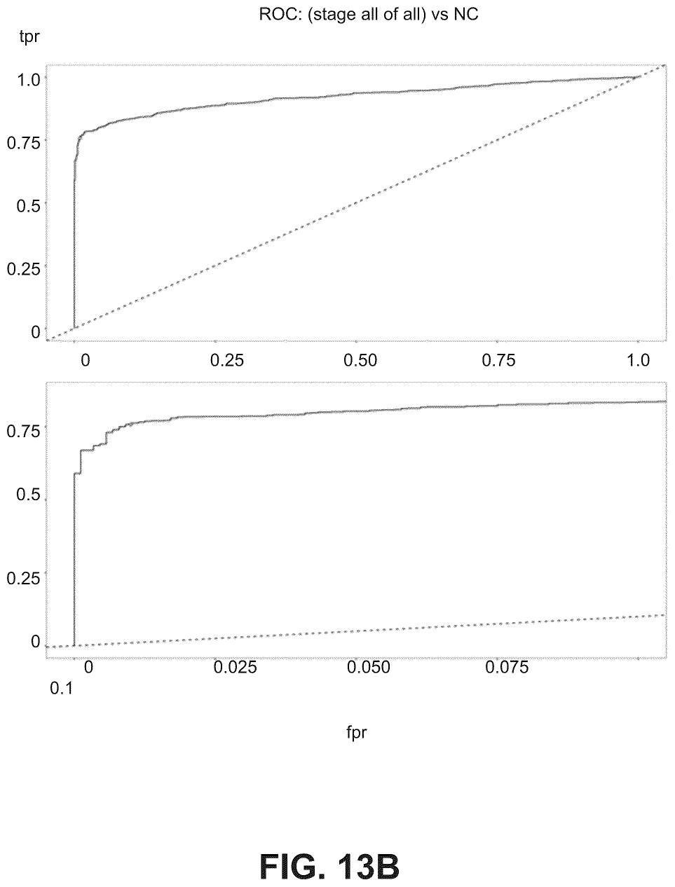

FIGS. 13A and 13B illustrates the receiver operating characteristic (ROC) curves for the tissue of origin classifier. The receiver operating characteristic (ROC) curves show classifier performance at 99% specificity with 55% sensitivity for all cancers and 76% sensitivity for multicancer.

These data show that classification methods using targeted methylation features simultaneously detected multiple cancer types, at early stages, at a specificity (99%) appropriate for population screening. Detection of multiple cancers was achieved with a single, fixed, low false positive rate. This approach also accurately localized the tissue of origin, which would streamline downstream diagnostic work-up. Additionally, incorporating data from a large methylation database improved performance of the classifier.

Together, this supports the potential clinical applicability of the method described in the present disclosure as an early multi-cancer detection test for numerous clinically significant cancer types.

### X.c. Example 3—Classification of Cancer Using Targeted Bisulfite Sequencing from Complete Second CCGA Sub-Study

Generation of a mixture model classifier: To maximize performance, the predictive cancer models described in this Example were trained using sequence data obtained from a plurality of samples from known cancer types and non-cancers from both CCGA sub-studies (CCGA1 and CCGA2), a plurality of tissue samples for known cancers obtained from CCGA1, and a plurality of non-cancer samples from the STRIVE study (See Clinical Trail.gov Identifier: NCT03085888 (//clinicaltrials.gov/ct2/show/NCT03085888)). The STRIVE study is a prospective, multi-center, observational cohort study to validate an assay for the early detection of breast cancer and other invasive cancers, from which additional non-cancer training samples were obtained to train the classifier described herein. The known cancer types included from the CCGA sample set included the following: breast, lung, prostate, colorectal, renal, uterine, pancreas, esophageal, lymphoma, head and neck, ovarian, hepatobiliary, melanoma, cervical, multiple myeloma, leukemia, thyroid, bladder, gastric, and anorectal. As such, a model can be a multi-cancer model (or a multi-cancer classifier) for detecting one or more, two or more, three or more, four or more, five or more, ten or more, or 20 or more different types of cancer. 4,841 participants (2,836 cancer; 2,005 non-cancer) from the CCGA study and 2,202 non-cancer participants from the STRIVE study were included in this pre-specified analysis. Of these, 3,133 samples from CCGA were allocated to training (1,742 cancer; 1,391 non-cancer) and 1,354 were allocated to validation (740 cancer, 614 non-cancer); 1,587 samples from STRIVE were allocated to training and 615 to validation. Participant disposition is indicated. Overall, 3,052 samples in training (1,531 cancer; 1,521 non-cancer) and 1,264 samples in validation (654 cancer; 610 non-cancer) were analyzable and in the pre-specified primary analysis population. Additional details on the CCGA2 substudy, and on the analysis detailed in this Example, were described in an Annals of Oncology journal article, entitled “Sensitive and specific multi-cancer detection and localization using methylation signatures in cell-free DNA,” which was published online on Mar. 30, 2020 (https:www.annalsofoncology.org/article/S0923-7534(20)36058-0/fulltext).

The classifier performance data shown below was reported out for a locked classifier trained on cancer and non-cancer samples obtained from CCGA2, a CCGA sub-study, and on non-cancer samples from STRIVE. The individuals in the CCGA2 sub-study were different from the individuals in the CCGA1 sub-study whose cfDNA was used to select target genomes (as described in WO 2019/195268 filed Apr. 2, 2019, PCT/US2019/053509 filed Sep. 27, 2019 and PCT/US2020/015082 filed Jan. 24, 2020 (which are incorporated herein by reference)). From the CCGA2 study, blood samples were collected from individuals diagnosed with untreated cancer (including 20 tumor types and all stages of cancer) and healthy individuals with no cancer diagnosis (controls). For STRIVE, blood samples were collected from women within 28 days of their screening mammogram. Cell-free DNA (cfDNA) was extracted from each sample and treated with bisulfite to convert unmethylated cytosines to uracils. The bisulfite treated cfDNA was enriched for informative cfDNA molecules using hybridization probes designed to enrich bisulfite-converted nucleic acids derived from each of a plurality of targeted genomic regions in three cancer assay panels: (1) pan-cancer assay panel #4 as described and disclosed in WO 2019/195268 (labeled herein as Assay Panel A herein); (2) pan-cancer assay panel #5 as described and disclosed in WO 2019/195268 (labeled herein as Assay Panel B herein); and (3) a large proprietary pan-cancer assay panel (Assay Panel C, described below). The enriched bisulfite-converted nucleic acid molecules were sequenced using paired-end sequencing on an Illumina platform (San Diego, CA) to obtain a set of sequence reads for each of the training samples, and the resulting read pairs were aligned to the reference genome, assembled into fragments, and methylated and unmethylated CpG sites identified.

Mixture Model Based Featurization

For each cancer type (including non-cancer) a probabilistic mixture model was trained and utilized to assign a probability to each fragment from each cancer and non-cancer sample based on how likely it was that the fragment would be observed in a given sample type.

Fragment-Level Analysis

Briefly, for each sample type (cancer and non-cancer samples), for each region (where each region was used as-is if less than 1 kb, or else subdivided into 1 kb regions in length with a 50% overlap (e.g., 500 base pairs overlap) between adjacent regions), a probabilistic model was fit to the fragments derived from the training samples for each type of cancer and non-cancer. The probabilistic model trained for each sample type was a mixture model, where each of three mixture components was an independent-sites model in which methylation at each CpG is assumed to be independent of methylation at other CpGs. Fragments were excluded from the model if: they had a p-value (from a non-cancer Markov model) greater than 0.01; were marked as duplicate fragments; the fragments had a bag size of greater than 1 (for targeted methylation samples only); they did not cover at least one CpG site; or if the fragment was greater than 1000 bases in length. Retained training fragments were assigned to a region if they overlapped at least one CpG from that region. If a fragment overlapped CpGs in multiple regions, it was assigned to all of them.

Local Source Models

Each probabilistic model was fit using maximum-likelihood estimation to identify a set of parameters that maximized the log-likelihood of all fragments deriving from each sample type, subject to a regularization penalty. Specifically, in each classification region, a set of probabilistic models were trained, one for each training label (i.e., one for each cancer type and one for non-cancer). Each model took the form of a Bernoulli mixture model with three components. Mathematically,

Pr(fragment|{βₖᵢ,fₖ})=Σₖ₌₁ⁿ fₖΠᵢ βₖᵢ^{m}^{ᵢ}(1−βₖᵢ)^{1-m}^{ᵢ}

where n is the number of mixture components, set to 3; mᵢ∈{0, 1} is the fragment's observed methylation at position i; fₖ is the fractional assignment to component k (with fₖ≥0 and Σfₖ=1); and βₖᵢ is the methylation fraction in component k at CpG i. The product over i included only those positions for which a methylation state could be identified from the sequencing. Maximum-likelihood values of the parameters {fₖ, βₖᵢ} of each model were estimated by using the rprop algorithm (e.g., the rprop algorithm as described in Riedmiller M, Braun H. RPROP—A Fast Adaptive Learning Algorithm. Proceedings of the International Symposium on Computer and Information Science VII, 1992) to maximize the total log-likelihood of the fragments of one training label, subject to a regularization penalty on βₖᵢ that took the form of a beta-distributed prior. Mathematically, the maximized quantity was

$${\sum }_{j} ln ( Pr ( {fragment}_{j} \vert \{ {β}_{ki} , {f}_{k} \} ) ) + {\sum }_{k , i} r ln ( {β}_{ki} ( 1 - {β}_{ki} ) )$$

where r is the regularization strength, which was set to 1.

Featurization

Once the probabilistic models were trained, a set of numerical features was computed for each sample. Specifically, features were extracted for each fragment from each training sample, for each cancer type and non-cancer sample, in each region. The extracted features were the tallies of outlier fragments (i.e., informative fragments), which were defined as those whose log-likelihood under a first cancer model exceeded the log-likelihood under a second cancer model or non-cancer model by at least a threshold tier value. Outlier fragments were tallied separately for each genomic region, sample model (i.e., cancer type), and tier (for tiers 1, 2, 3, 4, 5, 6, 7, 8, and 9), yielding 9 features per region for each sample type. In this way, each feature was defined by three properties: a genomic region; a “positive” cancer type label (excluding non-cancer); and the tier value selected from the set {1, 2, 3, 4, 5, 6, 7, 8, 9}. The numerical value of each feature was defined as the number of fragments in that region such that

$$ln ( \frac{Pr ( fragment \vert positive cancer type )}{Pr ( fragment \vert non - cancer )} ) > tier$$

where the probabilities were defined by equation (1) using the maximum-likelihood-estimated parameter values corresponding to the “positive” cancer type (in the numerator of the logarithm) or to non-cancer (in the denominator).

Feature Ranking

For each set of pairwise features, the features were ranked using mutual information based on their ability to distinguish the first cancer type (which defined the log-likelihood model from which the feature was derived) from the second cancer type or non-cancer. Specifically, two ranked lists of features were compiled for each unique pair of class labels: one with the first label assigned as the “positive” and the second as the “negative”, and the other with the positive/negative assignment swapped (with the exception of the “non-cancer” label, which was only permitted as the negative label). For each of these ranked lists, only features whose positive cancer type label (as in equation (3)) matched the positive label under consideration were included in the ranking. For each such feature, the fraction of training samples with non-zero feature value was calculated separately for the positive and negative labels. Features for which this fraction was greater in the positive label were ranked by their mutual information with respect to that pair of class labels.

The top ranked 256 features from each pairwise comparison were identified and added to the final feature set for each cancer type and non-cancer. To avoid redundancy, if more than one feature was selected from the same positive type and genomic region (i.e., for multiple negative types), only the one assigned the lowest (most informative) rank for its cancer type pair was retained, breaking ties by choosing the higher tier value. The features in the final feature set for each sample (cancer type and non-cancer) were binarized (any feature value greater than 0 was set to 1, so that all features were either 0 or 1).

Classifier Training

The training samples were then divided into distinct 5-fold cross-validation training sets, and a two-stage classifier was trained for each fold, in each case training on ⅘ of the training samples and using the remaining ⅕ for validation.

In the first stage of training, a binary (two-class) logistic regression model for detecting the presence of cancer was trained to discriminate the cancer samples (regardless of TOO) from non-cancer. When training this binary classifier, a sample weight was assigned to the male non-cancer samples to counteract sex-imbalance in the training set. For each sample, the binary classifier outputs a prediction score indicating the likelihood of a presence or absence of cancer.

In the second stage of training, a parallel multi-class logistic regression model for determining cancer tissue of origin was trained with TOO as the target label. Only the cancer samples that received a score above the 95th percentile of the non-cancer samples in the first stage classifier were included in the training of this multi-class classifier. For each cancer sample used in training the multi-class classifier, the multi-class classifier outputs prediction values for the cancer types being classified, where each prediction value is a likelihood that the given sample has a certain cancer type. For example, the cancer classifier can return a cancer prediction for a test sample including a prediction score for breast cancer, a prediction score for lung cancer, and/or a prediction score for no cancer.

Both binary and multi-class classifiers were trained by stochastic gradient descent with mini-batches, and in each case, training was stopped early when the performance on the validation fold (assessed by cross-entropy loss) began to degrade. For predicting on samples outside of the training set, in each stage, the scores assigned by the five cross-validated classifiers were averaged. Scores assigned to sex-inappropriate cancer types were set to zero, with the remaining values renormalized to sum to one.

Scores assigned to the validation folds within the training set were retained for use in assigning cutoff values (thresholds) to target certain performance metrics. In particular, the probability scores assigned to the training set non-cancer samples were used to define thresholds corresponding to particular specificity levels. For example, for a desired specificity target of 99.4%, the threshold was set at the 99.4th percentile of the cross-validated cancer detection probability scores assigned to the non-cancer samples in the training set. Training samples with a probability score that exceeded a threshold were called as positive for cancer.

Subsequently, for each training sample determined to be positive for cancer, a TOO or cancer type assessment was made from the multiclass classifier. First, the multi-class logistic regression classifier assigned a set of probability scores, one for each prospective cancer type, to each sample. Next, the confidence of these scores was assessed as the difference between the highest and second-highest scores assigned by the multi-class classifier for each sample. Then, the cross-validated training set scores were used to identify the lowest threshold value such that of the cancer samples in the training set with top-two score differential exceeding the threshold, 90% had been assigned the correct TOO label as their highest score. In this way, the scores assigned to the validation folds during training were further used to determine a second threshold for distinguishing between confident and indeterminate TOO calls.

At prediction time, samples receiving a score from the binary (first-stage) classifier below the predefined specificity threshold were assigned a “non-cancer” label. For the remaining samples, those whose top-two TOO-score differential from the second-stage classifier was below the second predefined threshold were assigned the “indeterminate cancer” label. The remaining samples were assigned the cancer label to which the TOO classifier assigned the highest score.

Classifier Performance on Target Genomic Region Panels

The discriminatory value of the target genomic regions of Assay Panels A-C was evaluated by testing the ability of a cancer classifier to detect cancer and any of 20 different cancer types according to the methylation status of these target genomic regions. For Assay Panels A-B, performance was evaluated over a training set of 1,531 cancer samples and 1,521 non-cancer samples that were used to train the classifier, as shown in TABLE 1. For Assay Panel C, performance was evaluated using 1,264 samples in validation (654 cancer; 610 non-cancer) on a classifier trained using the same set of 3,052 samples that were used in training for Assay Panels A-B (1,531 cancer; 1,521 non-cancer). For each sample, differentially methylated cfDNA was enriched using a bait set comprising all of the target genomic regions included in Assay Panels A-C. The classifier was then constrained to provide cancer determinations based only on the methylation status of the target genomic regions of the List being evaluated. A two-stage classifier embodiment including a binary (two-class) logistic regression classifier model for detecting the presence of cancer that was trained to discriminate the cancer samples (regardless of TOO) from non-cancer and a second stage trained a multi-class logistic regression classifier model for determining cancer tissue of origin was trained with TOO as the target label, as previously described in this Example. Also as previously described, both classifier models were trained and validated using model-based featurization

TABLE 1
Cancer diagnoses of individuals whose
cfDNA was used to train the classifier

| | | Stage | | | | |
|---|---|---|---|---|---|---|
| | | | | | | Not |
| Cancer Type | Total | I | II | III | IV | Reported |
| Non-cancer | 1521 | — | — | — | — | — |
| Lung | 261 | 60 | 23 | 72 | 106 | 0 |
| Breast | 247 | 102 | 110 | 27 | 8 | 0 |
| Prostate | 188 | 39 | 113 | 19 | 17 | 0 |
| Lymphoid neoplasm | 147 | 15 | 27 | 27 | 39 | 39 |
| Colorectal | 121 | 13 | 22 | 41 | 45 | 0 |
| Pancreas and gallbladder | 95 | 15 | 15 | 19 | 46 | 0 |
| Uterine | 84 | 73 | 3 | 5 | 3 | 0 |
| Upper GI | 67 | 9 | 12 | 19 | 27 | 0 |
| Head and neck | 62 | 7 | 13 | 16 | 26 | 0 |
| Renal | 56 | 37 | 4 | 4 | 11 | 0 |
| Ovary | 37 | 4 | 2 | 25 | 6 | 0 |
| Multiple myeloma | 34 | 10 | 13 | 11 | 0 | 0 |
| Not reported | 29 | 8 | 5 | 7 | 6 | 3 |
| Liver bile duct | 29 | 5 | 7 | 7 | 10 | 0 |
| Sarcoma | 17 | 2 | 4 | 5 | 6 | 0 |
| Bladder and urothelial | 16 | 6 | 7 | 3 | 1 | 0 |
| Anorectal | 14 | 4 | 5 | 5 | 0 | 0 |
| Cervical | 11 | 8 | 1 | 2 | 0 | 0 |
| Melanoma | 7 | 3 | 1 | 0 | 3 | 0 |
| Myeloid neoplasm | 4 | 2 | 1 | 0 | 1 | 0 |
| Thyroid | 4 | 0 | 0 | 0 | 0 | 4 |
| Prediction only | 2 | 0 | 0 | 0 | 2 | 0 |

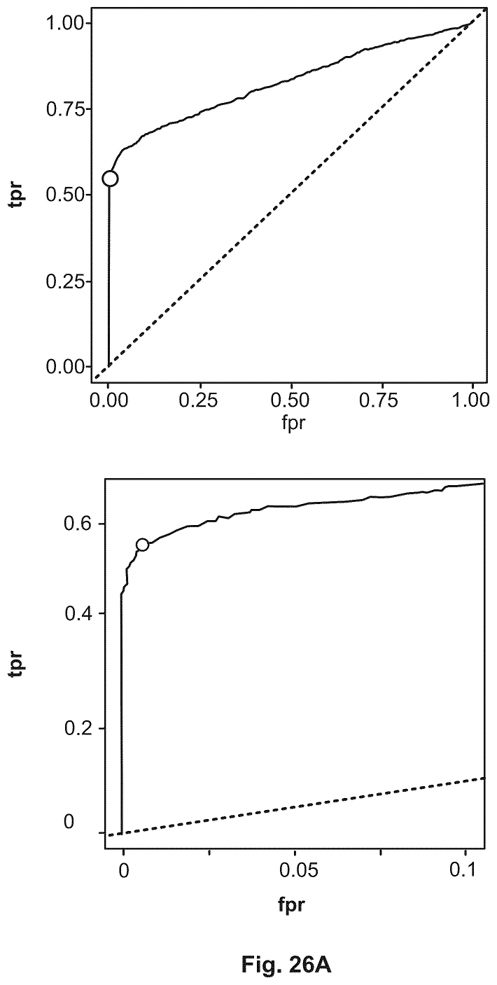

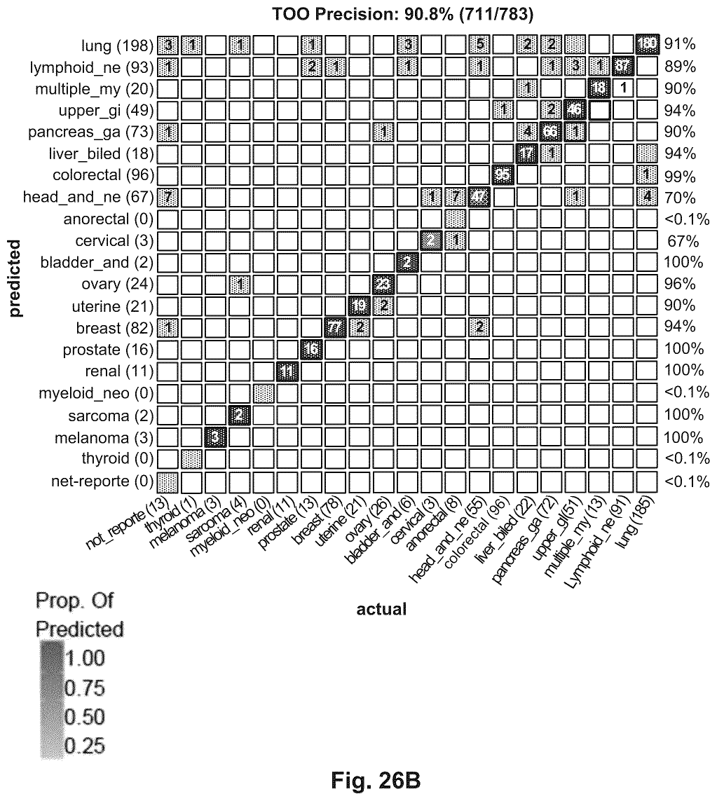

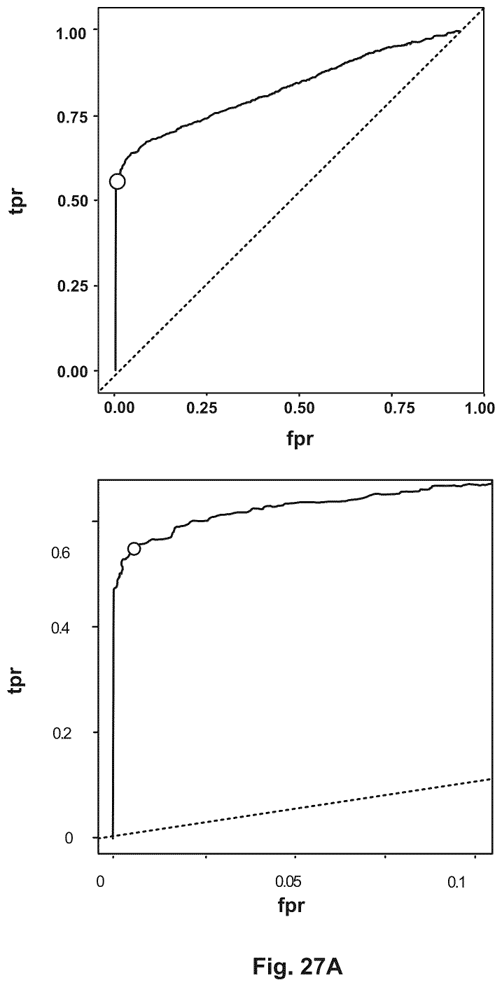

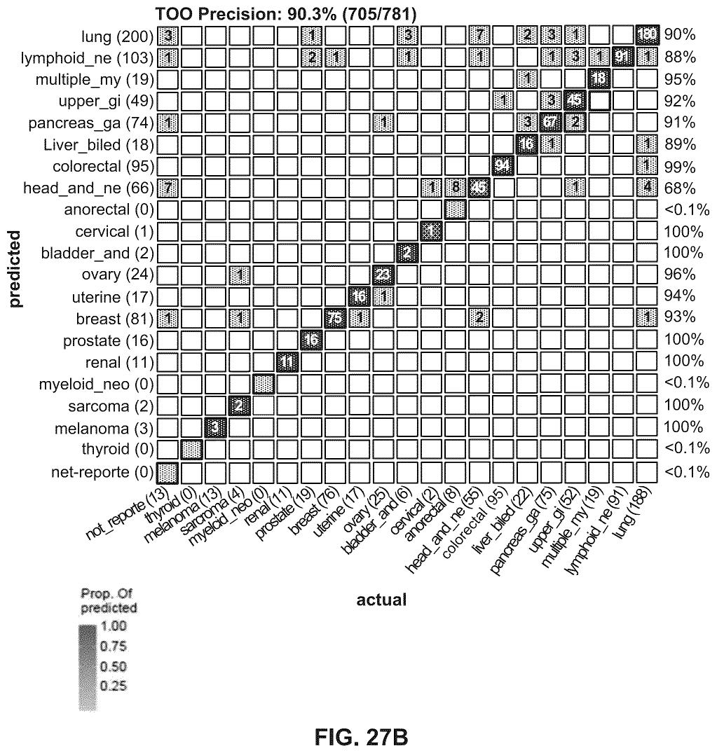

Assay Panels A and B: Results from the classifier performance analysis for Assay Panels A and B are presented in FIGS. 26A and 27A. In each figure, part A is a receiver operator curve (ROC) showing true positive results and false positive results for a determination of cancer or no-cancer. The asymmetric shape of these ROC curves illustrates that the classifier was designed to minimize false positive results. The areas under the curve for Assay Panels A and B was 0.83 for both assay panels.

A Cancer Type (i.e. TOO) determination was made using the classifier for all samples that tested positive for cancer. FIGS. 26B and 27B include confusion matrices indicating accuracy of TOO accuracy for Assay Panels A and B, respectively. The confusion matrix includes information describing a success rate for the classifier at identifying each of cancer types and excluding indeterminate cancer calls.

As shown in FIGS. 26B and 27B, the TOO confusion matrices demonstrate the performance for the multi-class logistic regression classifier, as described above. Agreement between the actual (x-axis) and predicted (y-axis) tissue of origin per sample using the targeted methylation classifier is depicted. Scores along the diagonal of the matrices indicate correct predictions, that is, where the predicted tissue of origin for a fragment matches the true tissue of origin. As shown in FIG. 26B, cancer Assay Panel A had a TOO accuracy of approximately 90.8% (711/783), when excluding indeterminate cancer calls. And FIG. 27B shows that Assay Panel B had a TOO accuracy of approximately 90.3% (705/781), when excluding indeterminate cancer calls.

These classifier results are further summarized in TABLES 2-3, which indicate the accuracy of cancer detections and cancer type determinations made with a specificity of 0.990, indicating a false positive rate of 1%. These results are delineated by cancer stage. They show improved cancer detection and cancer type determinations for samples from individuals with later stage cancers (e.g. stage III) compared to samples from individuals with earlier stage cancers (e.g. stage II). For all cancer stages (no segregation by stage), the cancer type determination was accurate approximately 89%, for both Assay Panels A and B (including indeterminate cancer calls).

TABLE 2
Classification accuracy using the genomic regions of Assay Panel A.
Data for Cancer Presence and Cancer type at a specificity of 0.990
show percentage accuracy, a 95% confidence interval in brackets,
and the number correctly assigned over the total in parentheses.

| Stage | Cancer Presence | Cancer Type |
|---|---|---|
| I | 20.4% [16.6-24.5] (86/422) | 71.8% [60.5-81.4] (56/78) |
| II | 44.6% [39.6-49.7] (173/388) | 87.2% [81.1-91.9] (143/164) |
| III | 81.5% [76.7-85.6] (255/313) | 90.5% [86.1-93.9] (220/243) |
| IV | 90.9% [87.5-93.7] (330/363) | 93.3% [90-95.8] (294/315) |
| All | 56.5% [54-59] (866/1532) | 89.1% [86.8-91.2] (731/820) |

TABLE 3
Classification accuracy using the genomic regions of Assay Panel B.
Data for Cancer Presence and Cancer type at a specificity of 0.990
show percentage accuracy, a 95% confidence interval in brackets,
and the number correctly assigned over the total in parentheses.

| Stage | Cancer Presence | Cancer Type |
|---|---|---|
| I | 19.9% [16.2-24] (84/422) | 72.7% [60.4-83] (48/66) |
| II | 45.1% [40.1-50.2] (175/388) | 84.8% [78.2-90] (134/158) |
| III | 81.2% [76.4-85.3] (254/313) | 91.3% [86.9-94.6] (211/231) |
| IV | 90.9% [87.5-93.7] (330/363) | 93.2% [89.8-95.7] (287/308) |
| All | 56.3% [53.7-58.8] (862/1532) | 89.2% [86.9-91.3] (697/781) |

Assay Panel C: As noted above, a third, large proprietary pan-cancer assay panel was also tested. Assay Panel C was designed using feature selection methods disclosed in PCT/US2019/053509 filed Sep. 27, 2019 and PCT/US2020/015082 filed Jan. 24, 2020 (which are incorporated herein by reference) from WGBS data obtained from the first CCGA sub-study, CCGA1. The large, proprietary targeted methylation panel, covered 103,456 distinct regions (17.2 Mb), covering 1,116,720 CpGs. Assay Panel C included 363,033 CpGs in 68,059 regions (7.5 Mb) covered by probes targeting hypomethylated fragments; 585,181 CpGs in 28,521 regions (7.4 Mb) covered by probes targeting hypermethylated fragments; and 218,506 CpGs in 6,876 regions (2.3 Mb) targeting both types of fragments. Individual abnormal target regions contained between 1 and 590 CpGs, with a median CpG count of 3 for hypomethylated target regions and 6 for hypermethylated target regions. CpGs were present in the following genomic regions: 193,818 (17%) in the region 1 to 5 kbp upstream of transcription start sites (TSSs); 278,872 (24%) in promoters (<1 kbp upstream of TSSs); 500,996 (43%) in introns; 292,789 (25%) in exons; 247,752 (21%) in intron-exon boundaries; 134,144(11%) in 5′-untranslated regions; 182,174 (16%) between genes; and the remaining 1,817 (<1%) were not annotated. Percentages were relative to the total number of CpGs and do not sum to 100% because each CpG could receive multiple annotations due to overlapping genes and/or transcripts.

For this evaluation samples were divided into training (n=4,720) and independent validation sets (n=1,969). A total of 4,316 participants (training: 3,052 [1,531 cancer: stage I: 28%; stage II: 25%; stage III: 20%; stage IV: 24%; missing/not expected: 3%; 1,521 non-cancer]; validation: 1,264 [654 cancer: stage I: 28%; stage II: 25%; stage III: 21%; stage IV: 23%; missing/not expected: 3%; 610 non-cancer]) were analyzable and included in the primary analysis population.

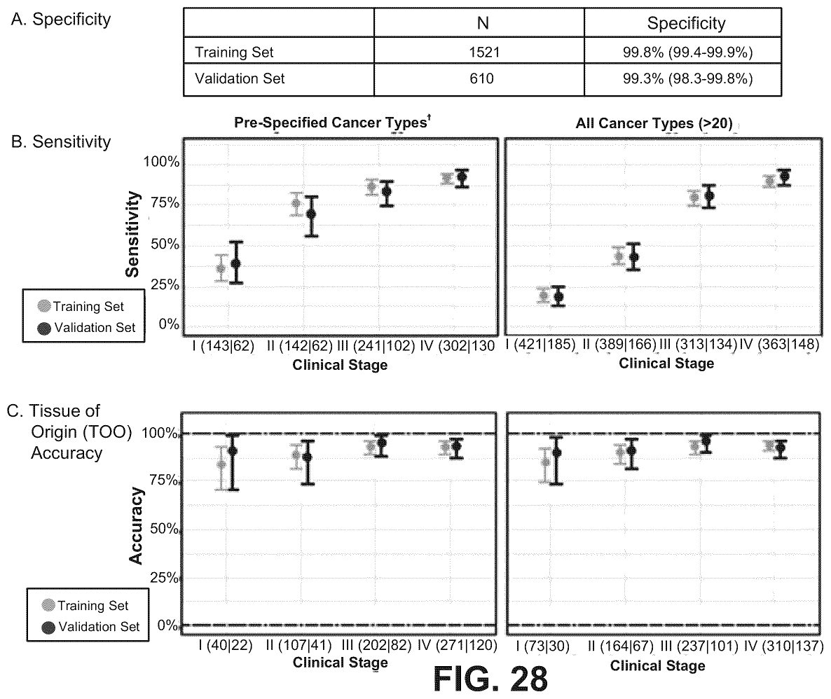

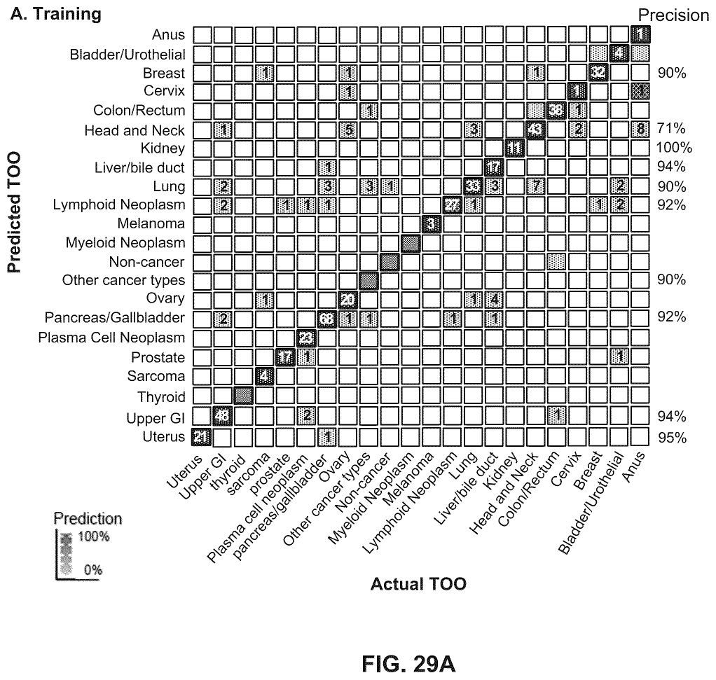

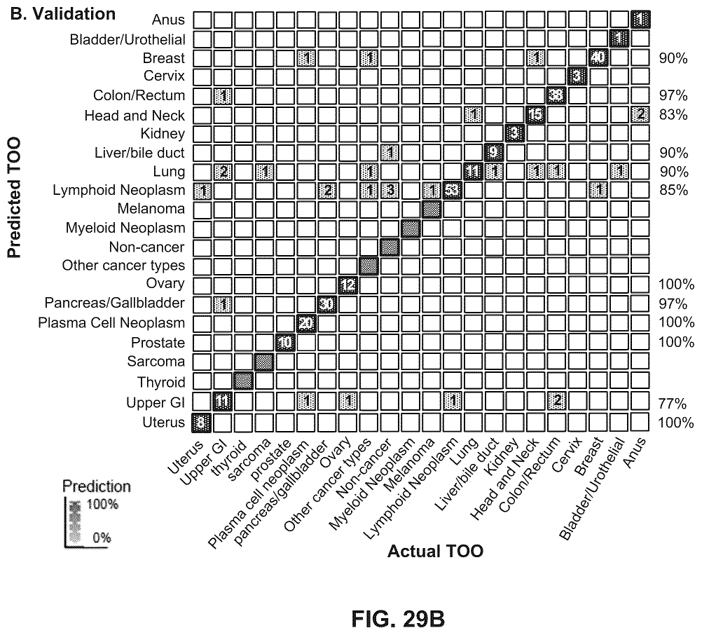

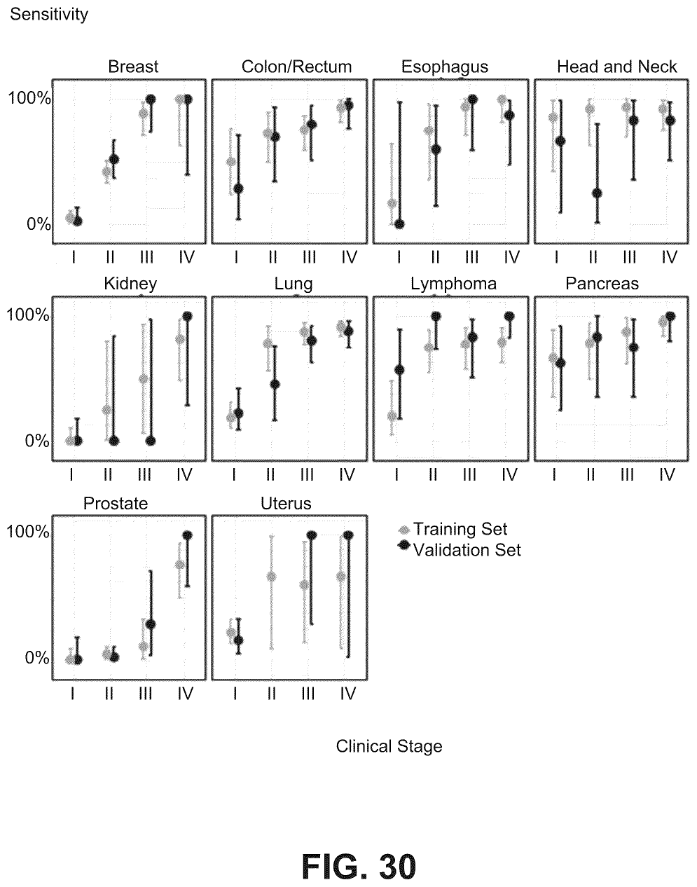

Results from the classifier performance analysis for the training and validation sets are shown in FIGS. 28-30. Panel A of FIG. 28 shows specificity results for both the training and validation sets, panel B shows sensitivity for pre-specified cancers (a subset of 12 high-signal cancers based on results from the first sub-study and mortality data (anus, bladder, colon/rectum, esophagus, head and neck, liver/bile-duct, lung, lymphoma, ovary, pancreas, plasma cell neoplasm, stomach)) and for all cancer types (>20) at stages I through IV. Panel C of FIG. 28 shows tissue of origin (TOO) accuracy results or both the training and validation sets, panel B shows sensitivity for pre-specified cancers and for all cancer types at stages I through IV. FIG. 29 shows TOO confusion matrices for both the training and validation sets and FIG. 30 shows sensitivity results for the pre-specified cancer types for both the training and validation sets.

In FIG. 28, sensitivity (y-axis) is reported by clinical stage (x-axis) in the pre-specified cancer types (left panel) and in all cancer types (right panel) for training (orange) and validation (teal). Tissue of origin accuracy (y-axis) is reported by clinical stage (x-axis) in the pre-specified cancer types (left panel) and in all cancer types (right panel) for training (orange) and validation (teal). Numbers indicate samples in training|validation sets.

As shown in FIG. 28, the classifier achieved consistently high specificity between the cross-validated training and independent validation sets (99.8% [95% CI: 99.4-99.9%] versus 99.3% [98.3-99.8%], respectively; P=0.095); this reflected a single, consistent, false positive rate (FPR) of less than 1% across all 20 cancer types. Specificity in the validation set was similar for the CCGA and STRIVE non-cancer samples (99.3% [97.4-99.9%] vs 99.4% [97.9-99.9%], respectively), supporting that performance was not biased by sites or selected samples. Sensitivity was consistent in the training and validation sets. In all cancers, stage I-III sensitivity was 44.2% (95% CI: 41.3-47.2%) versus 43.9% (39.4-48.5%) (P=1.000), respectively. For the pre-specified set of 12 high-signal cancers, stage I-III sensitivity was 69.8% (65.6-73.7%) versus 67.3% (60.7-73.3%), respectively (P=0.988). Similarly, stage I-IV sensitivity across all cancer types was 55.2% (52.7-57.7%) versus 54.9% (51.0-58.8%), respectively (P=0.897), and in the pre-specified cancers was 77.9% (75.0-80.7%) versus 76.4% (71.6-80.7%), respectively (P=0.573).

Also, as shown in FIG. 28, sensitivity increased with increasing stage of disease. In validation, sensitivity in pre-specified cancer types was 39% (27-52%) in stage I (n=62), 69% (56-80%) in stage II (n=62), 83% (75-90%) in stage III (n=102), and 92% (86-96%) in stage IV (n=130). Among all cancer types, sensitivity was 18% (13-25%) in stage I (n=185), 43% (35-51%) in stage II (n=166), 81% (73-87%) in stage III (n=134), and 93% (87-96%) in stage IV (n=148).

Performance in individual tumor types is depicted in FIG. 30. Sensitivity at 99.8% specificity (training, orange) or 99.3% specificity (validation, teal) with 95% confidence intervals is reported for individual cancer types with at least 50 samples. Clinical stage is indicated below the plots, as is the number of samples in training and validation.

As shown in FIG. 28, pre-specified analysis of TOO accuracy (the fraction of all TOO predictions that were correct) found that TOO was predicted in 96% (344/359) of samples with a cancer-like signal in the validation set; among these, accuracy was 93% (321/344). Accuracy was consistent between the training and validation sets and across stages. The classifier distinguished>20 cancer types included in the study, with consistent performance in individual cancer types.

FIG. 29 shows confusion matrices representing the accuracy of tissue of origin localization in the (A) training and (B) validation sets. Agreement between the actual (x-axis) and predicted (y-axis) tissue of origin per sample using the targeted methylation classifier is depicted. Color corresponds to the proportion of predicted tissue of origin calls. Included participants (training: n=844, validation: n=359) are those with cancer predicted as having cancer at 99.8% specificity (training) or 99.3% specificity (validation). The tissue of origin calls were assigned in 95% (806/844) of cases in training, and in 96% (344/359) of cases in validation; calls were correct in 92% (744/806) of cases in training and in 93% (321/344) of cases in validation.

### X.d. Example 4—Tuning of Binary Classification Threshold

According to generalized embodiment of binary cancer classification, the analytics system determines a cancer score for a test sample based on the test sample's sequencing data (e.g., methylation sequencing data, SNP sequencing data, other DNA sequencing data, RNA sequencing data, etc.). The analytics system compares the cancer score for the test sample against a binary threshold cutoff for predicting whether the test sample likely has cancer. The binary threshold cutoff can be tuned using TOO thresholding based on one or more TOO subtype classes. The analytics system may further generate a feature vector for the test sample for use in the multiclass cancer classifier to determine a cancer prediction indicating one or more likely cancer types.

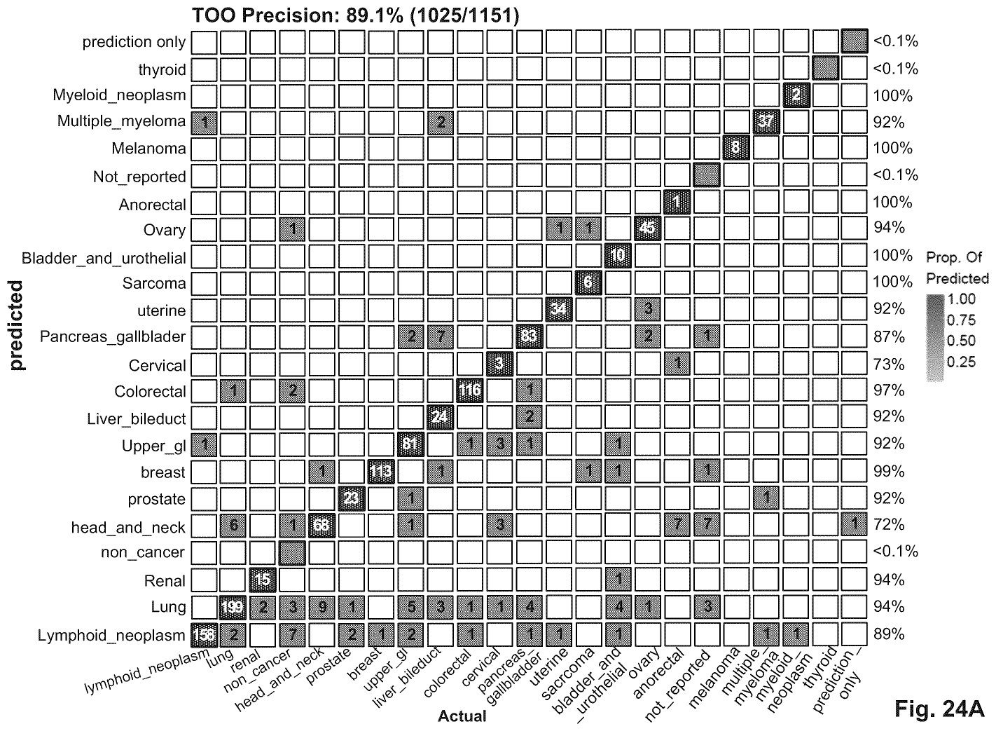

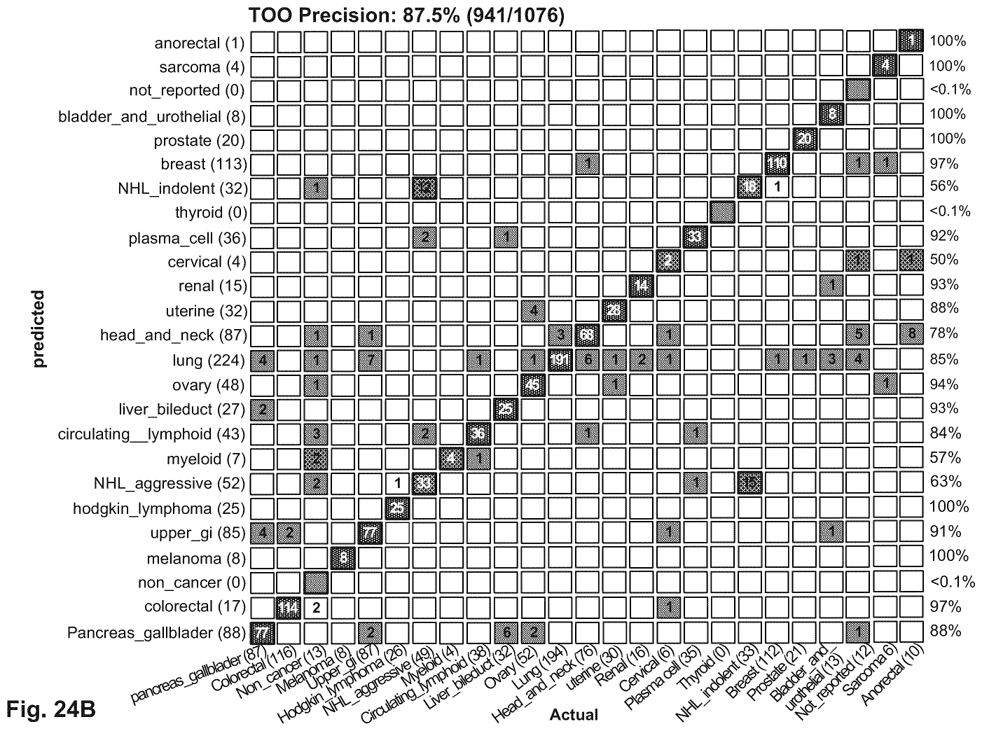

FIG. 24A illustrates a confusion matrix demonstrating performance of a trained cancer classifier, according to an example implementation. The cancer classifier was trained according to the principles described above. The TOO labels include: lymphoid neoplasm, lung, renal, non-cancer, head and neck, prostate, breast, upper gastrointestinal, liver and bile duct, colorectal, cervical, pancreas and gallbladder, uterine, sarcoma, bladder and urothelial, ovary, anorectal, unknown, melanoma, multiple myeloma, myeloid neoplasm, and thyroid. Of note, the classification precision is 89.1% over 1,151 samples considered in this holdout set.

FIG. 24B illustrates a confusion matrix demonstrating performance of a trained cancer classifier with additional hematological cancer sub-types. The cancer classifier was trained according to the principles described above. In contrast to FIG. 24A, the TOO labels for hematological sub-types have been adjusted. In FIG. 24A, the hematological sub-types include lymphoid neoplasm, multiple myeloma, and myeloid neoplasm. In FIG. 24B, the hematological sub-types include Hodgkin's-Lymphoma (HL), NHL aggressive, NHL indolent, myeloid, circulating lymphoma (or lymphoid), and plasma cell. Of note, the classification precision is 87.5% over 1,076.

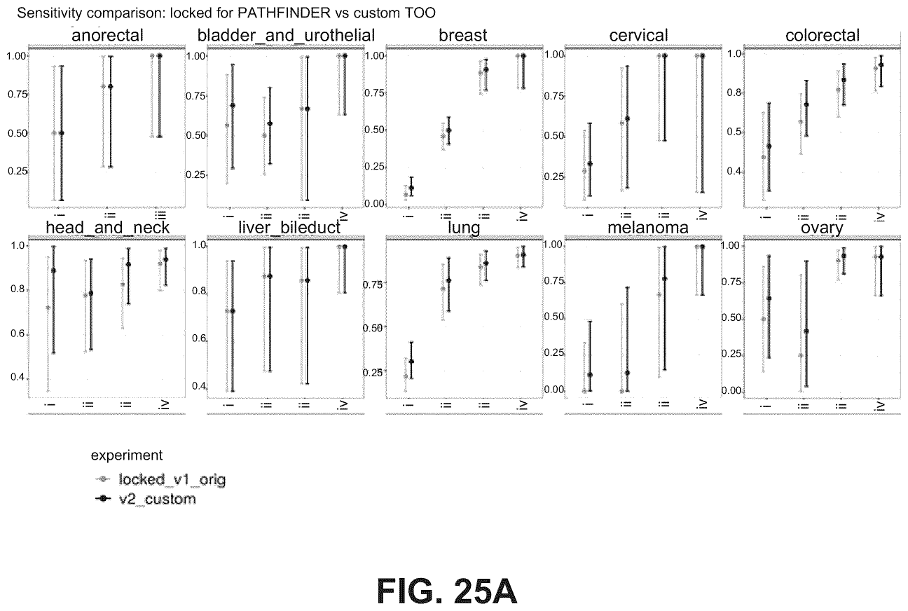

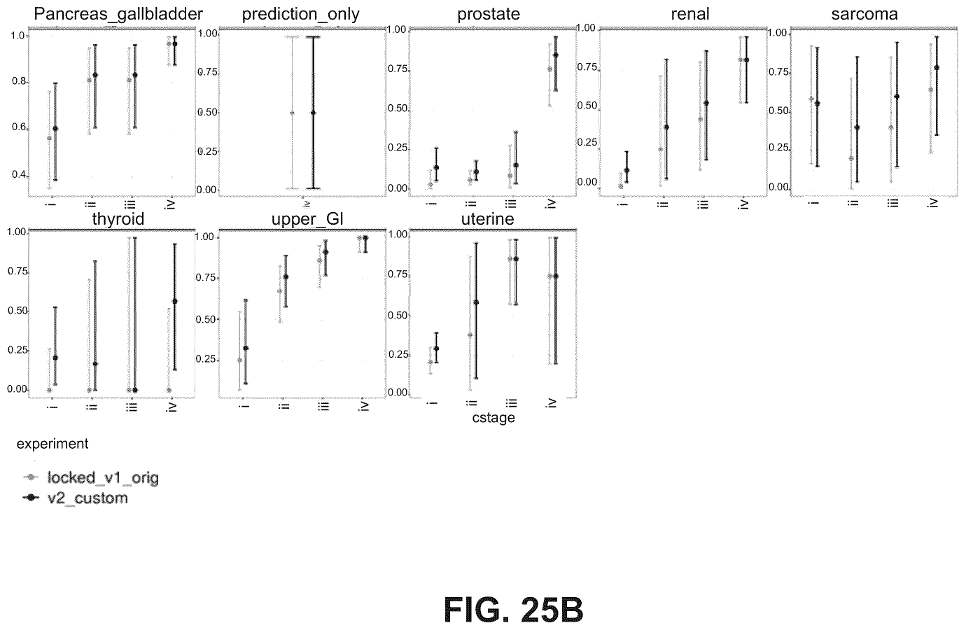

FIGS. 25A and 25B illustrate graphs showing cancer prediction accuracy for numerous cancer types over stages of cancer. In this example, the cancer classifier is trained after pruning the non-cancer samples according to the process 1000 described above. The analytics system determined multiple TOO thresholds for the hematological sub-types. The analytics system excluded non-cancer samples with at least one TOO probability at or above the corresponding TOO threshold for the hematological sub-types. The graphs shown show the classification sensitivity over varying stages of cancer for cancer types: anorectal, bladder and urothelial, breast, cervical, colorectal, head and neck, liver and bile duct, lung, melanoma, ovary, pancreas and gallbladder, prostate, renal, sarcoma, thyroid, upper gastrointestinal, and uterine. A graph for each cancer type shows the prediction sensitivity over each stage of the cancer type with a first cancer classifier without TOO thresholding labeled as “locked_v1_orgi” and a second cancer classifier with TOO thresholding labeled as “v2_custom”. Notably, for many cancer types the second cancer classifier has higher prediction accuracy while maintaining a tight confidence interval, given more samples available for validation. Of particular note, there are higher prediction accuracies in many cancer types at the stage I and II levels, indicating improved prediction potential with TOO thresholding in early stage cancers.

### XI. ADDITIONAL CONSIDERATIONS

The foregoing description of the embodiments of the disclosure has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the disclosure in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules can be embodied in software, firmware, hardware, or any combinations thereof.

Any of the steps, operations, or processes described herein can be performed or implemented with one or more hardware or software modules, alone or in combination with other devices. In some embodiments, a software module is implemented with a computer program product including a computer-readable non-transitory medium containing computer program code, which can be executed by a computer processor for performing any or all of the steps, operations, or processes described.

Embodiments can also relate to a product that is produced by a computing process described herein. Such a product can include information resulting from a computing process, where the information is stored on a non-transitory, tangible computer readable storage medium and can include any embodiment of a computer program product or other data combination described herein.

Finally, the language used in the specification has been principally selected for readability and instructional purposes, and it cannot have been selected to delineate or circumscribe the inventive subject matter. It is therefore intended that the scope of the invention be limited not by this detailed description, but rather by any claims that issue on an application based hereon. Accordingly, the disclosure of the embodiments herein is intended to be illustrative, but not limiting, of the scope of the invention, which is set forth in the following claims.

1. A method comprising:
obtaining a plurality of training samples from individuals each having one of a plurality of disease states and each associated with one of a plurality of labels for a covariate characteristic, wherein each training sample comprises methylation sequence reads for at least 1,000 cell-free deoxyribonucleic acid (cfDNA) fragments obtained from the individual;
generating a first set of training samples and a second set of training samples by subdividing the plurality of training samples;
generating, for each training sample, a feature vector based on the methylation sequence reads of the training sample by:
for each methylation sequence read of the training sample:
applying each of a plurality of tissue models to the methylation sequence reads to determine a likelihood that the methylation sequence read is informative of presence of one disease state associated with the tissue model,
assigning the methylation sequence read to one of the disease states with the highest likelihood output by the tissue models, and
determining the feature vector based on the methylation sequence reads assigned to each disease state;
training, with the feature vectors of the first set of training samples, a classifier to generate a signal vector based on an input feature vector, wherein the signal vector comprises a value for each disease state;
applying the classifier to the feature vector of each training sample in the second set of training samples to generate a signal vector for each training sample in the second set of training samples; and
for each label of the plurality of labels for the covariate characteristic, determining a cutoff threshold for each disease state based on the signal vectors for the training samples in the second set of training samples with the label.

2. The method of claim 1, wherein the methylation sequence reads are obtained from a targeted methylation sequencing assay, or a whole genome bisulfite sequencing assay.

3. The method of claim 1, wherein each training sample comprises methylation sequence reads for at least 10,000 cfDNA fragments.

4. The method of claim 1, wherein the first set of training samples and the second set of training samples comprise similar proportions of training samples over the disease states.

5. The method of claim 1, wherein the plurality of disease states includes a non-cancer state and one or more cancer states for one or more cancers of distinct origins.

6. The method of claim 5, wherein the one or more cancers of distinct origins comprise: breast cancer, uterine cancer, cervical cancer, ovarian cancer, bladder cancer, urothelial cancer of renal pelvis and ureter, renal cancer other than urothelial, prostate cancer, anorectal cancer, colorectal cancer, squamous cell cancer of esophagus, esophageal cancer other than squamous, gastric cancer, hepatobiliary cancer arising from hepatocytes, hepatobiliary cancer arising from cells other than hepatocytes, pancreatic cancer, human-papillomavirus-associated head and neck cancer, head and neck cancer not associated with human papillomavirus, lung adenocarcinoma, small cell lung cancer, squamous cell lung cancer and lung cancer other than adenocarcinoma or small cell lung cancer, neuroendocrine cancer, melanoma, thyroid cancer, sarcoma, multiple myeloma, lymphoma, and leukemia.

7. The method of claim 1, wherein the covariate characteristic is one of: age, status as a smoker, and biological sex.

8. The method of claim 1, further comprising:
determining, for each methylation sequence read, with p-value filtering whether the methylation sequence read has an informative methylation pattern, wherein the feature vector for each training sample is generated based on the methylation sequence reads with informative methylation patterns.

9. The method of claim 1, wherein each tissue model of the plurality of tissue models is trained by:
obtaining a second plurality of training samples each having one of a plurality of disease states, wherein each training sample comprises methylation sequence reads for cfDNA fragments obtained from an individual;
for each tissue model, generating a training dataset comprising at least 10,000 methylation sequence reads of training samples having the disease state associated with the tissue model; and
training each tissue model with the associated training dataset to predict a likelihood that a methylation sequence read is informative of presence of the associated disease state.

10. The method of claim 1, wherein each tissue model is one of: a binomial model, an independent sites model, a Markov model, or a mixture model.

11. The method of claim 1, wherein the classifier is a machine-learning model.

12. The method of claim 1, further comprising:
obtaining a test sample from a test individual having an unknown disease state and associated with a first label of the plurality of labels for the covariate characteristic, wherein the test sample comprises methylation sequence reads for cfDNA fragments obtained from the test individual;
generating a test feature vector based on the methylation sequence reads of the test sample by:
for each methylation sequence read of the test sample:
applying each of the plurality of tissue models to the methylation sequence reads to determine a likelihood that the methylation sequence read is informative of presence of one disease state associated with the tissue model,
assigning the methylation sequence read to one of the disease states with the highest likelihood output by the tissue models, and
determining the test feature vector based on the methylation sequence reads assigned to each disease state;
applying the classifier to the test feature vector of the test to generate a signal vector for the test sample; and
detecting positive disease signal for one or more of the disease states by applying the cutoff thresholds associated with the first label for the covariate characteristic to the signal vector for the test sample.

13. The method of claim 12, wherein detecting the positive disease signal for one or more of the disease states comprises:
for each disease state, applying the cutoff threshold for the disease state to the value in the signal vector for the test sample corresponding to the disease state.

14. The method of claim 12,
wherein each individual is further associated with one of a second plurality of labels for a second covariate characteristic;
wherein determining the cutoff thresholds comprises determining, for each combination of one label from the plurality of labels for the covariate characteristic and one label from the second plurality of labels for the second covariate characteristic;
wherein the test sample is further associated with a second label for the second covariate characteristic; and
wherein detecting the positive disease signal for one or more of the disease states comprises applying the cutoff thresholds associated with the combination of the first label for the covariate characteristic and the second label for the second covariate characteristic.

15. The method of claim 12, further comprising:
reporting the positive disease signal to a healthcare provider for additional workup diagnostic steps.

16. A method comprising:
obtaining a plurality of training samples from individuals each having one of a plurality of disease states and each associated with one of a plurality of labels for a covariate characteristic, wherein each training sample comprises methylation sequence reads for at least 1,000 cell-free deoxyribonucleic acid (cfDNA) fragments obtained from the individual;
generating, for each training sample, a feature vector based on the methylation sequence reads of the training sample by:
for each methylation sequence read of the training sample:
applying each of a plurality of tissue models to the methylation sequence reads to determine a likelihood that the methylation sequence read is informative of presence of one disease state associated with the tissue model,
assigning the methylation sequence read to one of the disease states with the highest likelihood output by the tissue models, and
determining the feature vector based on the methylation sequence reads assigned to each disease state;
generating a set of training samples for each label of the plurality of labels for the covariate characteristic comprising the feature vectors of the training samples associated with the label for the covariate characteristic; and
for each label, training, with the feature vectors of the set of training samples corresponding to the label, a classifier to generate a signal vector based on an input feature vector, wherein the signal vector comprises a value for each disease state.

17.-25. (canceled)

26. The method of claim 16, further comprising:
obtaining a test sample from a test individual having an unknown disease state and associated with a first label of the plurality of labels for the covariate characteristic, wherein the test sample comprises methylation sequence reads for cfDNA fragments obtained from the test individual;
generating a test feature vector based on the methylation sequence reads of the test sample by:
for each methylation sequence read of the test sample:
applying each of the plurality of tissue models to the methylation sequence reads to determine a likelihood that the methylation sequence read is informative of presence of one disease state associated with the tissue model,
assigning the methylation sequence read to one of the disease states with the highest likelihood output by the tissue models, and
determining the test feature vector based on the methylation sequence reads assigned to each disease state;
applying the classifier corresponding to the first label to the test feature vector of the test to generate a signal vector for the test sample; and
detecting positive disease signal for one or more of the disease states based on the signal vector for the test sample.

27.-29. (canceled)

30. A method comprising:
obtaining a plurality of training samples from individuals each having one of a plurality of disease states and each associated with one of a plurality of labels for a covariate characteristic, wherein each training sample comprises methylation sequence reads for at least 1,000 cell-free deoxyribonucleic acid (cfDNA) fragments obtained from the individual;
generating, for each training sample, a feature vector based on the methylation sequence reads of the training sample by:
for each of a plurality of genomic regions, determining a methylation feature value based on one or more methylation sequence reads of the training sample overlapping the genomic region, and
determining the feature vector based on the methylation feature values across the plurality of genomic regions;
generating a set of training samples for each label of the plurality of labels for the covariate characteristic comprising the feature vectors of the training samples associated with the label for the covariate characteristic; and
for each label:
determining a mutual information score for each genomic region based on the feature vectors of the set of training samples corresponding to the label;
ranking the genomic regions based on the mutual information scores;
selecting a set of features from the ranked genomic regions;
modifying the feature vectors to include methylation feature values for the set of features; and
training a classifier with the modified feature vectors to generate a signal vector based on an input feature vector, wherein the signal vector comprises a value for each disease state.

31.-64. (canceled)
